(19) United States
(12) Patent Application Publication
(10) Pub. No.: US 2024/0325776 A1
(43) Pub. Date: Oct. 3, 2024

(54) NEUROMODULATION TECHNIQUES AND DEVICES RELATING THERETO

(71) Applicant: Trustees of Boston University, Boston, MA (US)

(72) Inventors: Xue Han, Chestnut Hill, MA (US); Sudiksha Sridhar, Boston, MA (US); Sanaya Ness Shroff, Brookline, MA (US); Eric Lowet, Huizen (NL); Howard Gritton, Mahomet, IL (US); Jennifer Freire, Roxbury, MA (US)

(73) Assignee: Trustees of Boston University, Boston, MA (US)

(21) Appl. No.: 18/621,968

(22) Filed: Mar. 29, 2024

Related U.S. Application Data
(60) Provisional application No. 63/455,361, filed on Mar. 29, 2023.

(51) Int. Cl. A61N 5/06

(52) U.S. Cl. CPC A61N 5/0613; A61N 2005/0626

(57) ABSTRACT

Techniques and devices for improving rhythmic motion of a subject are described herein. In particular, the disclosed techniques and devices utilize stimuli (acoustic, visual, tactile, and/or vibrational stimuli) delivered at beta frequencies and/or gamma frequencies to enhance neural activity and movement at delta frequencies in the subject. The disclosed techniques and devices may be used to improve motor symptoms in subjects with neurodegenerative conditions and/or to promote athletic training in healthy individuals.

### CROSS-REFERENCE TO RELATED APPLICATIONS

This application claims priority to and the benefit of U.S. Provisional Patent Application No. 63/455,361, filed Mar. 29, 2023, entitled “Novel Neuromodulation Techniques and Uses Thereof,” the contents of which are incorporated herein by reference as though fully set forth herein.

### GOVERNMENT SUPPORT

This invention was made with government support under Contract Nos. MH122971, NS 115797, and NS 115421 awarded by the National Institutes of Health and Contract No. 1848029 awarded by the National Science Foundation. The government has certain rights in the invention.

### FIELD OF THE TECHNOLOGY

The subject disclosure relates to neuromodulation techniques, and particularly to methods of improving physical motor function of a subject using audio, visual, and/or tactile stimuli.

### BACKGROUND

There are numerous known neurodegenerative movement disorders, such as Parkinson's disease, multiple system atrophy, progressive supranuclear palsy, and Huntington's disease. These and other neurodegenerative movement disorders can impair physical movement, especially rhythmic movement. Stepping movement is highly rhythmic and occurs at delta frequencies (0.5-4 Hz) in humans and mice.

Rhythmic neural network activity has been broadly linked to behavior. However, it is unclear how the neural circuits track behavioral rhythms, even though many neurons exhibit pace-making properties in isolated brain circuits. While the spinal central pattern generators are critical for pacing movement, stepping related neural activities are broadly detected in many motor-related brain areas, including the motor cortex, dorsal striatum, subthalamic nucleus (STN), and cerebellum. Stepping, as with many aspects of voluntary movement, is intricately linked to sensory inputs and sensory cues with similar rhythms to the pace of a given physical activity. Sensory cues delivered at a similar frequency to a human walking pace/cadence have been used as a compensation strategy to improve gait in Parkinsonian patients. Indeed, rhythmic auditory cues, such as metronome, music with adapted tempo or beats, or counting, when played around individual subject's stepping frequencies have been shown to improve patients' gait and mobility.

### SUMMARY

As summarized above, the neural mechanisms controlling rhythmic activities are not well understood. Currently, the only known effective method of improving rhythmic functioning in subjects with neurodegenerative movement disorders is to provide the subject with a pace-making sensory input having the desired cadence as the subject attempts to perform the physical activity at the desired cadence. In light of the challenges with regulating physical movement in individuals with movement disorders and the uncertainty surrounding how neural circuits impact behavioral rhythms, in the present disclosure, devices and techniques for improving rhythmic motion in a subject are described.

In some aspects, methods of improving gait in a subject are disclosed. The methods include exposing the subject to one or more acoustic stimuli having a frequency of 5 Hz to 30 Hz while the subject is engaging in an activity involving a repetitive physical motion performed at a frequency of less than 5 Hz. In some embodiments, the one or more acoustic stimuli have a frequency of 8 Hz to 14 Hz. In these and other embodiments, the methods also include restricting acoustic stimuli at frequencies less than 5 Hz and greater than 30 Hz from the subject while the subject is exposed to the one or more acoustic stimuli. The one or more acoustic stimuli may be delivered to the subject via headphones. The activity may be walking or another activity involving a repetitive physical motion. The physical motion may be performed at a frequency of approximately 0.5-4 Hz. The method may decrease a level of variation in gait of the subject by at least 5%, or in some cases, by at least 10%. The level of variation in gait may be measured by comparing a difference in at least one of: velocity, stride length, stride width, cadence, gait phases, and electrical activity produced by muscles in the subject. The subject may be a mammal, for example, a human. In select embodiments, the one or more acoustic stimuli may be delivered to the subject in a pattern in which there is a first period of time with acoustic stimuli followed by a second period of time without the acoustic stimuli. In some such embodiments, the first period of time may be equal to the second period of time or unequal to the second period of time. In certain implementations, during the second period of time, no acoustic stimuli are delivered to the subject. The disclosed methods may be used in connection with a subject who has been diagnosed with a neurodegenerative disease, such as multiple sclerosis, essential tremor, amyotrophic lateral sclerosis, and/or Parkinson's disease. In these and other embodiments, the methods may be used in connection with a subject suffering from: one or more myelopathies, spinal amyotrophy, cerebellar ataxia, brain tumor, craneoencephalic trauma, neuromuscular disease, one or more cerebrovascular pathologies, dementia, heart disease, and/or physiological ageing.

In another aspect, methods of improving neural activity in a subject are described. The methods include exposing the subject to audio, visual, tactile, and/or vibrational sensory stimuli at beta frequencies and/or gamma frequencies to promote neural activity in the subject at delta frequencies. In some embodiments, the subject may be exposed to the audio and/or visual stimuli while the subject is engaging in an activity involving a repetitive physical motion performed at delta frequencies. In these and other embodiments, the beta frequencies are from 10 Hz-35 Hz, the gamma frequencies are greater than 35 Hz, and/or the delta frequencies are from 0.5 Hz-4 Hz. In some embodiments, the delta frequencies are coordinated with cadence or stepping cycles of the subject. The disclosed methods may promote locomotion of the subject. The methods may decrease a level of variation in gait of the subject by at least 5%. The level of variation in gait may be measured by comparing a difference in at least one of: velocity, stride length, stride width, cadence, gait phases, and electrical activity produced by muscles in the subject. The methods may be used in connection with a subject who does not have a diagnosed neurodegenerative condition and, in some such circumstances, the methods may promote athletic training in the subject. In other implementations, the methods may be used in connection with a subject who has been diagnosed with Parkinson's disease and the methods improve motor function and/or cognitive function of the subject.

In yet another aspect, devices for improving locomotion of a user are described. The devices include a speaker configured to deliver auditory stimuli to the user, a light source configured to deliver visual stimuli to the user, and a tactile stimulator configured to deliver vibrational sensory stimulation to the user. The auditory stimuli, the visual stimuli, and the vibrational sensory stimulation may be delivered at beta frequencies and/or gamma frequencies to promote gait through enhancing neural activity of the user at delta frequencies. In some embodiments, the methods may also include one or more sensors configured to measure a locomotion pattern of the user and a software package arranged to receive information from the one or more sensors regarding the locomotion patterns of the user and to generate a stimulation protocol based on the locomotion pattern. The speaker may include one or more headphones, if desired.

A reading of the following detailed description and a review of the associated drawings will make apparent the advantages of these and other inventive features of the present disclosure. Both the foregoing general description and the following detailed description serve as an explanation only and do not restrict aspects of the disclosure as claimed.

### BRIEF DESCRIPTION OF THE DRAWINGS

So that those having ordinary skill in the art to which the disclosure pertains will more readily understand how to make and use the disclosed subject matter, reference may have made to the following drawings.

FIGS. 1A-1C illustrate a sample gait pattern for a human, with FIG. 1A illustrating the measured acceleration magnitude over time, FIG. 1B illustrating a detailed view of a complete cycle of acceleration magnitude, and FIG. 1C illustrating a view of the gait pattern;

FIGS. 2A-2E illustrate plots of measured variance difference in gait across human subjects exposed to audio stimulation of varying frequencies;

FIGS. 3A-3B illustrate the effects of audio stimulation at 10 Hz on gait variance difference of a human subject;

FIGS. 4A-4R illustrate experimental data obtained from mice exposed to audio stimulation at frequencies of 10 Hz and 145 Hz;

FIGS. 5A-5K illustrate experimental data of mouse subjects exposed to audio stimulation at frequencies of 10 Hz and 145 Hz;

FIGS. 6A-6K illustrate experimental data relating to gait rhythmicity for mouse subjects exposed to audio stimulation at frequencies of 10 Hz and 145 Hz;

FIGS. 7A-7G illustrate experimental data relating to striatal neural activity for mice exposed to audio stimulation at various frequencies;

FIGS. 8A-8E illustrate experimental data relating to neural activity for mice exposed to audio stimulation at various frequencies;

FIGS. 9A-9M illustrate experimental data relating to activity of individual striatal neurons coupling to delta rhythmic movement speed and population striatal circuit activity measured as local field potentials for mice exposed to audio stimulation at various frequencies;

FIGS. 10A-10K illustrate experimental data relating to neuronal responses to sensory stimuli for mice exposed to audio stimulation at frequencies of 10 Hz and 145 Hz;

FIGS. 11A-11D illustrate experimental data relating to movement-responsive neuronal responses for mice exposed to audio stimulation at various frequencies;

FIGS. 12A-12B illustrate experimental data relating to movement-responsive neuron activation for mouse subjects exposed to audio stimulation at frequencies of 10 Hz and 145 Hz;

FIGS. 13A-13G illustrate experimental data relating to population of movement-responsive neurons during locomotion and sensory stimulation for mouse subjects exposed to audio stimulation at various frequencies;

FIGS. 14A-14F illustrate experimental data relating to movement-responsive neurons for mouse subjects exposed to audio stimulation at various frequencies;

FIGS. 15A-15D illustrate experimental data relating population response measured as correlation coefficient in shuffles as compared to calcium event onsets across neuron pairs in mouse subjects when exposed to audio stimulation at various frequencies;

FIGS. 16A-16P illustrate membrane voltage imaging of individual striatal neurons (SYNs) and cholinergic interneurons (ChI) observed in mice subjects during locomotion;

FIGS. 17A-17C illustrate fluorescence data of neurons expressing syn-SomArchon in dorsal striatal brain slices of mice subjects;

FIGS. 18A-18D illustrate experimental data relating to delta rhythmic SYN and non-delta rhythmic ChI;

FIG. 19 illustrates categorization of delta-rhythmic and non-delta neurons by inter-spike interval ratio;

FIGS. 20A-20Q illustrate experimental data relating to subthreshold membrane voltage (Vm) and spiking delta rhythm structures;

FIGS. 21A-21C illustrate experimental data relating to phase-locking value of spikes to image displacement for delta-rhythmic or non-delta neurons;

FIGS. 22A-22K illustrate experimental data relating to the coupling of subthreshold Vm delta timing and LFP beta power;

FIGS. 23A-23D illustrate experimental data relating to Vm delta-phase dependent beta power as compared to spiking activity;

FIGS. 24A-24H illustrate experimental data relating to locomotion-dependent spiking of delta-rhythmic and non-delta neurons;

FIGS. 25A-25H illustrate experimental data of firing rates during movement compared to rest periods for the ChI population and the SYN population;

FIGS. 26A-26F illustrate experimental data of the response of delta-rhythmic and non-delta neurons to movement onset;

FIGS. 27A-27F illustrate experimental data for spike-aligned LFP power during rest versus movement;

FIGS. 28A-28B illustrate experimental data of the firing rate at locomotion onset and locomotion offset; and

FIG. 29 illustrates experimental data of LFP rhythmicity and power during the stepping cycle of a mouse subject.

### DETAILED DESCRIPTION

Without wishing to be bound by theory, it is believed that neural activity at delta frequencies (0.5-4 Hz) in the basal ganglia is coordinated with stepping cycles, and sensory stimulation (audio and visual) at different frequencies can differentially alter basal ganglia neural dynamics and locomotion. Furthermore, it is believed that basal ganglia delta rhythm is coupled to higher frequency beta (˜10-35 Hz) and gamma oscillations (>35 Hz) that have been broadly implicated in Parkinson's disease. Based on experimental data described below, sensory stimulation at beta and gamma frequencies (10 Hz and 145 Hz specifically) has been shown to both promote locomotion, with beta frequency stimulation resulting in improved gait rhythmicity and sustained locomotion. Further, the experimental data from recording neural activity in mice has provided the circuit mechanisms for this behavioral improvement observed in human subjects, which is via engaging delta-rhythmic neural activity underlying gait regulation through enhancing beta frequency dynamics at the single neuron level. Together, these results support a novel neurornodulation strategy using stimulation patterns containing different frequency components to optimally promote desired locomotion features. The design of these stimulation patterns can be achieved using coupled oscillator biophysical principles, and an optimal stimulation pattern can be evolved through iterative experimental testing for maximum engagement of specific neural circuit dynamics. This disclosure also contemplates the use of intelligent stimulation patterns, which can be used to improve motor symptoms in Parkinson's disease patients and to promote athletic training in healthy individuals.

Although there are several ongoing clinical trials attempting to evaluate the effect of sensory stimulation on Parkinson's disease (P)), including auditory cuing, these clinical trials primarily focus on delta frequency stimulation. For example, some ongoing trials aim to examine the effect of music on alleviating PD-specific problems by regulating stepping patterns (see, for example, ClinicalTrials.gov Identifier: NCT05421624). Interestingly, there is one trial examining the effect of 40 Hz sensory stimulation on cognitive benefit in PD patients (see ClinicalTrials.gov Identifier: NCT05268887). In contrast to these approaches, the present disclosure relates to using intelligent stimulation patterns containing beta and gamma frequencies that promote neuronal encoding of the brain regions involved in locomotion, so that the stimulation patterns can optimally engage the neuronal responses to achieve better locomotion outcomes.

Techniques and devices for improving rhythmic motion of a subject are described herein. In particular, the disclosed techniques and devices utilize stimuli (acoustic, visual, tactile, and/or vibrational stimuli) delivered at beta frequencies and/or gamma frequencies to enhance neural activity at delta frequencies in the subject. The disclosed techniques and devices may be used to improve motor symptoms in subjects with neurodegenerative conditions and/or to promote athletic training in healthy individuals.

In certain aspects, a device is described. The device includes: (1) a software package that generates sensory stimulation patterns that are synthesized based on neuronal biophysical properties, (2) one or more sensors that measure locomotion patterns, (3) one or more speakers that deliver auditory stimuli, (4) a light source that delivers visual stimuli, and/or (5) a tactile stimulator that delivers vibrational sensory stimuli. In some implementations, the device includes any combination of the following sensory stimulation modalities: auditory, visual, and tactile stimulation. In embodiments, the device includes a speaker (e.g., a headphone or other type of speaker) configured to deliver auditory stimuli to a user; a light source configured to deliver visual stimuli to the user, and a tactile stimulator configured to deliver vibrational sensory stimulation to the user. The auditory stimuli, visual stimuli, and the vibrational sensory stimulation are delivered at beta frequencies and/or gamma frequencies to promote gait through enhancing neural activity of the user. In select embodiments, the device also includes one or more sensors configured to measure a locomotion pattern of the user and a software package arranged to receive information from the one or more sensors regarding the locomotion patterns of the user and to generate a stimulation protocol based on the locomotion pattern.

In other aspects, methods of improving movement of a subject are described. The methods may be used to improve and/or regulate any type of movement, including rhythmic movements such as walking, running, or talking. In certain embodiments, the movement involves a repetitive physical motion performed at a frequency of approximately 0.5-4 Hz. The methods may include exposing the subject to one or more acoustic stimuli having a frequency of 5 Hz to 30 Hz while the subject is engaging in an activity involving a repetitive physical motion performed at a frequency of less than 5 Hz. The one or more acoustic stimuli may have a frequency of 8 Hz to 14 Hz. The methods may also include, in some implementations, restricting acoustic stimuli at frequencies less than 5 Hz and greater than 30 Hz from the subject while the subject is exposed to the one or more acoustic stimuli having a frequency of 5 Hz to 30 Hz.

The acoustic stimuli may be delivered to the subject via headphones (e.g., noise-cancelling headphones). In some embodiments, the acoustic stimuli may be delivered in a pattern in which there is a first period of time with acoustic stimuli followed by a second period of time without the acoustic stimuli (or without any acoustic stimuli). The first period of time may be equal to the second period of time or unequal to the second period of time. In some implementations, the first period of time is between 0.5-10 seconds and the second period of time is between 0.5-10 seconds.

The methods described herein may improve movement of the human or animal subject. Movement improvement may be quantitatively and/or qualitatively measured. In some embodiments, the methods may decrease variation in gait of the subject. Numerous methods of evaluating gait are known to those skilled in the art. For example, gait cadence, rhythmicity, velocity, stride length, gait phases, and electrical muscle activity may be evaluated to evaluate gait. The disclosed methods may decrease a level of variation in gait by at least 5%, or at least 10%, in some embodiments.

The methods may be used in connection with healthy subjects or with subjects who have been diagnosed with a neurodegenerative disease. For example, the methods may be used with subjects suffering from multiple sclerosis, essential tremor, amyotrophic lateral sclerosis, Parkinson's disease, one or more myelopathies, spinal amyotrophy, cerebellar ataxia, brain tumor, craneoencephalic trauma, neuromuscular disease, one or more cerebrovascular pathologies, dementia, heart disease, and/or physiological ageing.

In other aspects, the disclosure relates to methods of improving neural activity in a human or animal subject. The methods involve, in some embodiments, exposing the subject to audio, visual, tactile, and/or vibrational sensory stimuli at beta frequencies and/or gamma frequencies to promote neural activity in the subject at delta frequencies. The subject may be exposed to the audio and/or visual stimuli while the subject is engaging in an activity involving a repetitive physical motion performed at delta frequencies. The beta frequencies may be from 10 Hz-35 Hz, the gamma frequencies may be greater than 35 Hz, and the delta frequencies may be from 0.5 Hz-4 Hz.

These and other methods may be used to promote locomotion of the subject. In such embodiments, the delta frequencies may coordinate with cadence or stepping cycles of the subject. In select implernentations, the methods may decrease a level of variation in gait of the subject by at least 5% as measured by comparing a difference in at least one of: velocity, stride length, stride width, cadence, gait phases, and electrical activity produced by muscles in the subject. These and other methods described herein may be used in a subject who does not have a diagnosed neurodegenerative condition to promote athletic training in the subject or in a subject with a neurodegenerative condition (e.g., Parkinson's disease) to prove motor function and/or cognitive function of the subject.

Rhythmic neural activities are broadly observed across neural circuits and linked to behavior. Thus far, behaviorally relevant neural rhythms have been mainly studied by measuring electrical field potentials at the population circuit level. One particular rhythm, the delta (0.5-4 Hz) rhythm, is prominent not only at the circuit level, but also at the behavioral level. Neural circuit delta frequency oscillations occur across various motor circuits and are thought to provide a network coordination mechanism during locomotion. Interestingly, stepping movements also occur at delta frequencies in vertebrates including humans and mice. Locomotion depends on the central pattern generators in the spinal cord that exhibit autonomous pacemaking activity to orchestrate muscle movements, which are extensively modulated by supra-spinal descending inputs. The frequency similarity between neuronal delta rhythmicity and stepping cycles suggests that supra-spinal motor circuit activity may regulate movement patterning via frequency-dependent circuit coupling. Indeed, neurons in the motor cortex and the cerebellum exhibit stepping-relatedspiking activity at delta frequencies and sensory stimulation at delta frequencies improves gait and mobility in Parkinsonian patients. However, it is unknown whether similar movement-related delta-rhythmic temporal patterns occur in the striatum, the major input nucleus of the basal ganglia and a key structure for precise motor control that reciprocally interacts with the motor cortex and other subcortical motor circuits.

Delta rhythmic neural activity, typically measured as local field potential (LFP) delta oscillations, has been shown to organize higher frequency LFP oscillations at beta (˜20-40 Hz) and gamma (>35 Hz) frequencies across the cortico-basal ganglia-thalamic circuit. While prominent in layered cortical structures, LFP oscillations in the striatum are generally weak due to the lack of neuronal electrical dipole configurations. Nonetheless, transient fluctuations in striatal LFP beta oscillations have been related to different aspects of motor behavior and are coordinated by cortical and thalamic delta oscillations. Exaggerated beta oscillations, in particular, are considered a functional biomarker for Parkinson's disease.

So far, examination of the subthreshold membrane voltage (Vm) of individual striatal neurons has been limited to anesthetized animals, because of the difficulty of performing intracellular recordings during behavior. It therefore remains unclear how the Vm of different striatal neuron subtypes with distinct biophysical properties are dynamically modulated during movement. Among various striatal neuron subtypes, cholinergic interneurons (ChIs) likely play a prominent role in regulating striatal network dynamics and rhythmicity during movement due to their unique intrinsic autonomous pacemaking properties and their ability to influence the striatal network through their extensive anatomical arborizations. Further, ChIs exhibit movement-related activities and are also broadly implicated in Parkinson's disease, particularly in the pathophysiology of gait, a delta rhythmic movement pattern. To probe how cellular dynamics in the dorsal striatum contribute to rhythmic locomotion, we performed simultaneous voltage imaging of individual striatal neurons' Vm and spiking, and LFP recordings in mice during voluntary movement. We imaged membrane voltage at the soma of ChIs expressing the genetically-encoded, soma-targeted voltage indicator SomArchon, and compared ChI responses to that of non-specific striatal neurons, dominated by striatal spiny projection neurons (SPNs).

Stepping movement is delta (1-4 Hz) rhythmic and depends on sensory inputs. Since beta (10-30 Hz) rhythms are prominent in the motor circuits and are coupled to neuronal delta rhythms both at the network and the cellular levels, beta-frequency sensory stimulation influence on motor circuit regulation of stepping was evaluated in various experimental examples described below.

Beta frequency (10-30 Hz) oscillations are broadly observed across the cortical-basal ganglia circuits and related to sensorimotor processing. During locomotion, transient movement-related LFP beta bursts, hundreds of milliseconds long, are prominent across the motor cortex, striatum, globus pallidus externus, and subthalamic nucleus (STN). However, persistent, and exaggerated beta oscillations are widely recognized as a functional biomarker of akinesia and bradykinesia in Parkinson's disease. Thus, beta oscillations are hypothesized to maintain status quo, as transient beta also occurs during motor planning without actual movement, and inhibition or desynchronization of beta promotes movement transitions. Consistent with the anti-kinetic effects of beta rhythms, transcranial alternating current stimulation (tACS) at beta frequencies inhibits motor learning acquisition, slows voluntary movement, and reduces force generation in humans. Similarly, beta-frequency deep brain stimulation (DBS) in the STN may lead to further deterioration of bradykinesia and rigidity in Parkinsonian patients.

Dorsal striatum, the largest input structure of the basal ganglia, receives broad inputs from cortical and subcortical sensory and motor areas. Striatum plays important roles in various aspects of motor control and motor learning and is responsive to sensory stimulation. Individual striatal neurons exhibit diverse responses during sensorimotor tasks or naturalistic behaviors. At the network level, recent large-scale cellular calcium imaging analyses revealed that functional connectivity between clusters of striatal neurons could increase during specific actions or movement, even though the overall striatal network correlation strength reduces, highlighting how subpopulations of striatal neurons could be dynamically recruited during motor behaviors.

The coupling of delta and beta LFP oscillations are broadly observed across the cortico-basal ganglia-thalamic circuits. In the striatum, transient LFP beta oscillations have been reported during task initiation, cue processing, and movement completion, and fluctuation of beta power was found to be coupled to cortical and thalamic neuronal delta oscillations. Intriguingly, striatal beta was also found to be temporally locked to internally generated delta rhythmic finger tapping behavior, suggesting that neuronal and behavioral delta rhythms can temporally organize beta rhythmicity. Recently, using cellular voltage imaging, it was discovered that the membrane potentials of many striatal neurons exhibit prominent delta oscillations, which organize beta-rhythmic spike bursting and striatal LFP beta oscillations, highlighting a potential cellular mechanism for the observed circuit level presence of beta and delta coupling.

While strong stimulation of the motor circuits at beta frequencies using tACS or DBS results in anti-kinetic effects, sensory stimulation at beta frequencies could boost motor circuit processing of stepping movement via sensory entrainment, without producing exaggerated beta oscillations that are known to be anti-kinetic. To test this hypothesis, audiovisual stimulation at 10 Hz were delivered to mice voluntarily locomoting, since low frequency stimulations, sensory, tACS, DBS, and transcranial magnetic stimulation (TMS) have been found to entrain endogenous neural circuit oscillations. The effect of 10 Hz sensory stimulation was compared to the higher frequency 145 Hz stimulation that would be less effective in entraining neural circuits. The relationships between cellular calcium dynamics, functional connectivity between simultaneously recorded neurons, population striatal LFPs, and stepping movement were examined.

### Experimental Results

### Example 1-10 Hz Audio Stimulation Improved Gait Rhythmicity Across Human Populations

To examine the effect of beta frequency sensory stimulation, gait patterns of individual human subjects were tested both with and without auditory stimuli at 5-30 Hz. The auditory stimulus was a sine wave at a frequency of 5, 10, 20, or 30 Hz, played for 1 second on and 1 second off. Subjects walked for 20 meters per trial while listening to nothing (baseline), or an auditory stimulus of a particular frequency (stimulation). Gait was recorded from the right leg during all trials using a tri-axial accelerometer.

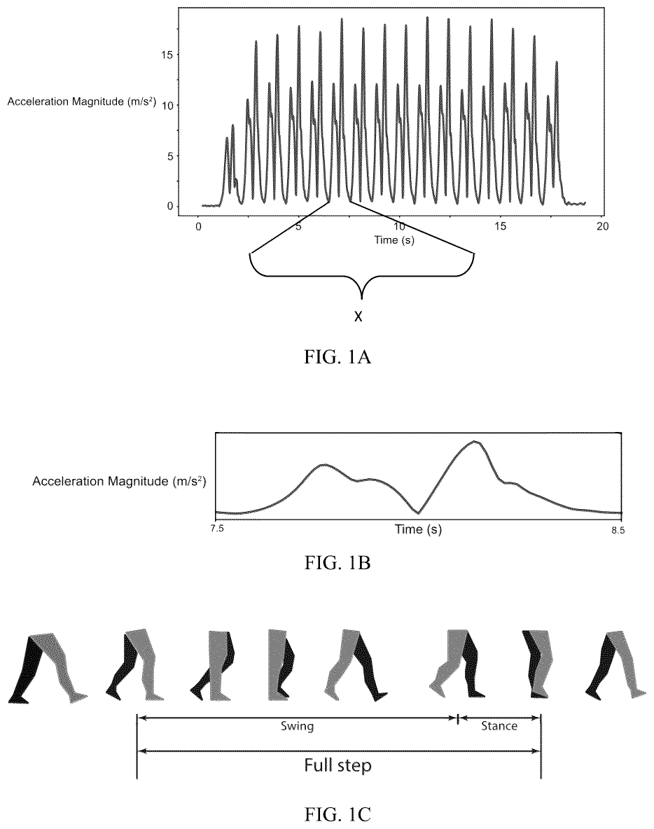

FIGS. 1A-1C illustrate the results of one sample individual's gait pattern. FIG. 1A shows a sample recording of leg movement acceleration while a subject walked for 20 meters, capturing multiple gait cycles. FIG. 1B illustrates a detailed view of the acceleration profile of a single full step cycle. FIG. 1C illustrates silhouetted leg positions corresponding to the swing and stance phases of the full step shown in FIG. 1B. Each full step contained a swing phase and a stance phase (see FIG. 1C), with characteristic acceleration magnitude. To characterize gait rhythmicity, each full step was identified using a custom threshold of acceleration magnitude, and the full step duration was computed as the time intervals between successive peaks. The initial and final four steps of each trial were excluded as subjects accelerated and decelerated during these times. The variance of full step duration was computed to measure step-to-step regularity or gait rhythmicity.

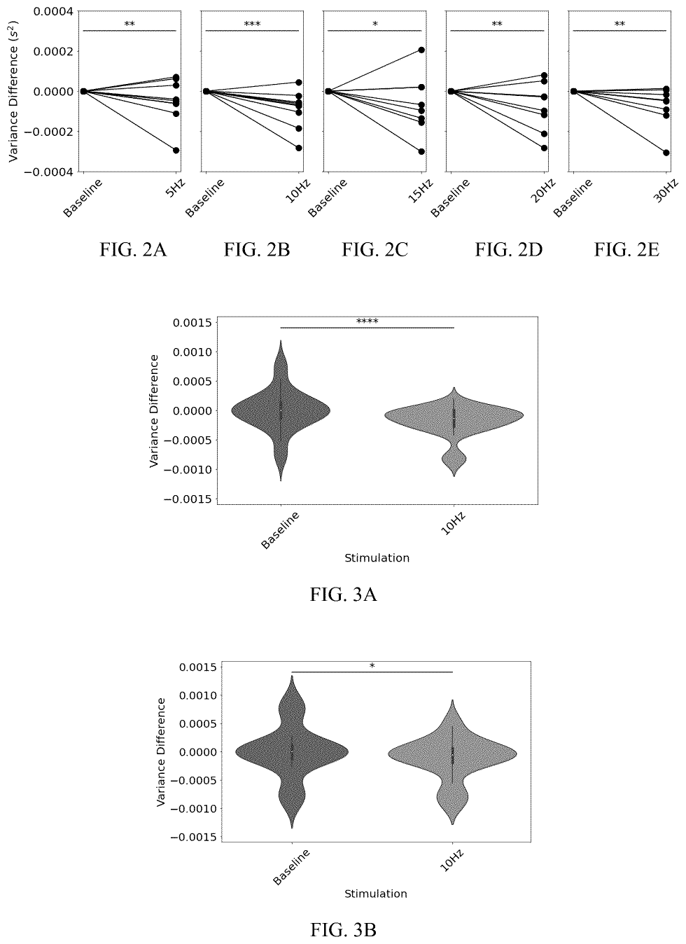

In this experiment, 9 human subjects were used. Each subject completed 4 trials of walking without audio stimulation (baseline) followed by 4 trials with audio stimulation at 5, 10, 20, or 30 Hz. Then the mean variance of step duration, a measure of gait rhythmicity, during baseline versus stimulation of a particular frequencies was compared. It was discovered that 8 (out of the 9) subjects showed a reduction in variance when listening to stimulation at all frequencies, with the most prominent improvement at 10 Hz with an average improvement rate of 40.72% (see FIGS. 2A-2E). Thus, auditory stimulation around beta frequencies improved gait rhythmicity.

FIGS. 2A-2E illustrate the effects of beta frequency audio stimulation. In particular, each dot in FIGS. 2A-2E represents the normalized step duration variance of an individual during baseline and audio stimulation at 5, 10, 20, and 30 Hz (n=23 total sessions from 9 subjects 10 Hz; n=21 total sessions from 9 subjects for 5 Hz and15 Hz; n=15 total sessions from 8 subjects for 20 Hz, 30 Hz). Statistical significance levels are denoted as *, p<0.05; **, p<0.01; ***, p<0.001 (Linear Mixed Effects Model). FIGS. 2A-2E show that 10 Hz audio stimulation consistently improved gait rhythmicity in many individuals.

### Example 2-10 Hz Audio Stimulation Consistently Improved Gait Rhythmicity in Many Individuals

After observing an improvement of gait pattern across populations, and the most prominent effect of 10 Hz audio stimulation, the consistency of gait improvement during 10 Hz stimulation in individuals was tested. Each individual performed 10 trials of baseline and 10 trials of audio stimulation, fully randomized. Of the 5 subjects tested, 3 showed consistent reduction in step duration variance, with a 38.14% reduction. Two such subjects are represented in FIGS. 3A-3B. These results further highlight that 10 Hz stimulation improves gait rhythmicity, enhances walking efficiency and stability.

FIGS. 3A-3B illustrate that 10 Hz audio stimulation consistently improved gait rhythmicity in many individuals. FIG. 3A illustrates a box plot of data from a first individual and FIG. 3B shows box plot data from a second individual. FIGS. 3A and 3B show a consistent reduction in step duration variance during 10 Hz stimulation compared to baseline. Each dot is the normalized step duration variance of an individual during baseline, and audio stimulation at 10 Hz (n=200 steps during baseline and stimulation, respectively, for both subjects.). *, p<0.05; **, p<0.01; ***, p<0.001; ***, p<0.0001, (Linear Mixed Effects Model).

### Example 3—Mouse Experimentation with 10 Hz Audio Stimulation

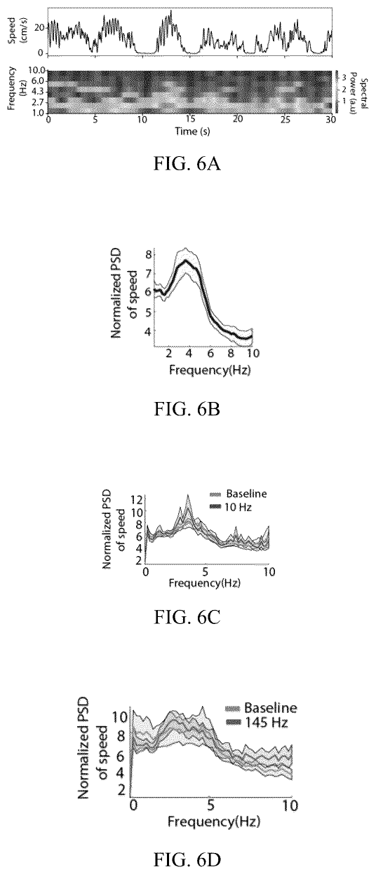

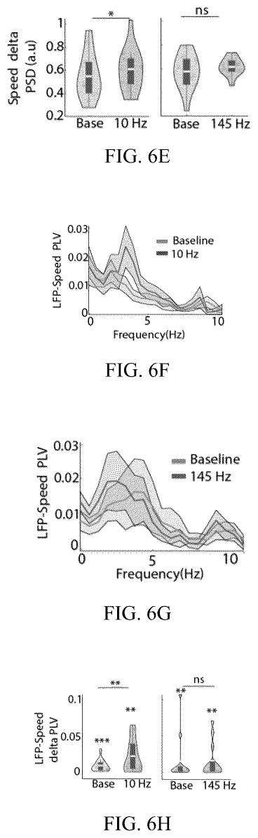

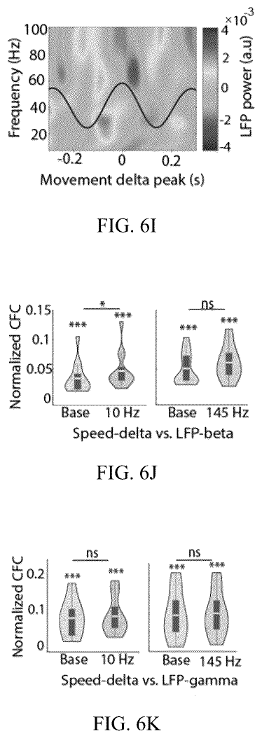

Experiments were also conducted on mice to assess the effect of audio stimulation on gait characteristics. FIGS. 6A-6K illustrate data obtained from experimentation with mice. Upon 10 Hz stimulation, the animal's movement became more rhythmic around delta frequencies (3-4 Hz), showing a significant increase in the relative delta power normalized across frequencies. The change in 3-4 Hz power was ˜27%.

As locomotion in mice and humans occurs in the delta frequency range, the spectral power of the treadmill speed was calculated that tracked animal's stepping cycles. Consistent with previous observations under similar locomotion conditions, mice stepping occurred at delta frequencies with a peak around 3-4 Hz (FIGS. 6A-6B). Upon 10 Hz stimulation, animal's movement became more rhythmic around delta frequencies (3-4 Hz), showing a significant increase in the relative delta power normalized across frequencies (FIGS. 6C, 6E). In contrast, 145 Hz stimulation did not alter movement delta rhythmicity (FIGS. 6D-6E).

FIG. 6A shows example movement speed trace (top) and corresponding power spectrogram (bottom) showing the delta rhythms. FIG. 6B shows power spectral density (PSD) of movement speed during running normalized to resting across all recording periods. Center line corresponds to the mean across all recording sessions, and shading, the standard error of the mean (n=28 recording sessions). FIG. 6C shows data similar to FIG. 6B, but for 10 Hz stimulation sessions only, with baseline in blue and 10 Hz stimulation in red. FIG. 6D is the same as FIG. 6C, but for 145 Hz stimulation sessions. FIG. 6E shows quantification of speed delta (3-4 Hz) PSD across all running bouts during baseline vs. 10 Hz or 145 Hz stimulation (Wilcoxon signed rank test, 10 Hz, p=0.01, n=14 sessions; 145 Hz: p=0.17, n=14 sessions). FIG. 6F shows LFP-movement speed PLV across frequencies during baseline (blue) vs. 10 Hz stimulation (red). FIG. 6G illustrates the same data as FIG. 6F, but for 145 Hz stimulation.

FIG. 6H shows LFP—movement speed delta PLV during baseline vs. 10 Hz or 145 stimulation (Wilcoxon signed rank test, 10 Hz: baseline vs. shuffle, p=2.4e⁻⁴, stimulation vs. shuffle: p=0.001, baseline vs. stimulation: p=0.008, n=13; 145 Hz: baseline vs. shuffle: p=0.002, stimulation vs. shuffle: p=0.006, baseline vs. stimulation: p=0.74, n=13). FIG. 6H shows a heatmap showing the relative LFP power aligned to the peak of the delta-frequency component of movement speed. FIG. 6J shows cross frequency coupling (CFC) between movement speed delta and beta-filtered LFP (15-30 Hz) during baseline vs. 10 Hz or 145 Hz stimulation (Wilcoxon signed rank test, 10 Hz: baseline: p=2.4e⁻⁴, 10 Hz: p=2.4e⁻⁴, baseline vs 10 Hz: p=0.03; 145 Hz: baseline: p=2.4e⁻⁴, 145 Hz: p=2.4e⁻⁴, baseline vs 145 Hz: p=0.24). FIG. 6K shows CFC between movement delta and gamma-filtered LFP during baseline vs. 10 Hz or 145 Hz stimulation (Wilcon signed rank test, 10 Hz: baseline: p=2.4e⁻⁴, 10 Hz: p=2.4e⁻⁴, baseline vs. 10 Hz: p=0.45; 145 Hz: baseline: p=2.4e⁻⁴, 145 Hz: p=2.4e⁻⁴, baseline vs. 145 Hz: p=0.73). Lines represent the mean across recording sessions and shaded regions are the standard error of the mean. Quantifications are visualized as violin plots with the outer shape representing the data kernel density and a box plot (box: interquartile range, whiskers: 1.5× interquartile range, white line: mean). *p<0.05, **p<0.01, ***p<0.001.

After detecting a change in stepping rhythmicity during 10 Hz stimulation, sensory stimulation influences the temporal relationship between striatal population dynamics and stepping were examined by computing the phase-locking value (PLV) between LFPs and movement speed. During running bouts, in the absence of sensory stimulation, there was a significant phase locking between LFPs and movement speed at delta frequencies (FIGS. 6F-6H), consistent with previous findings. Interestingly, 10 Hz stimulation, but not 145 Hz stimulation, further promoted LFP-movement speed phase locking at delta frequencies during running (FIGS. 6F-6H).

Our previous experimentation also showed that striatal LFP beta and gamma power are nested within the delta-rhythmic stepping cycles, peaking at a similar phase as spikes in delta-rhythmic striatal neurons. To understand how sensory stimulation influences the relationship between higher frequency components of the LFP power and stepping movement, the movement speed trace in the broader delta frequency range (2-4 Hz) was filtered and the mean LFP spectrogram was computed around all movement delta-peaks. Consistent with previous observations, in the absence of stimulation, LFP beta and gamma power (40-100 Hz) were modulated by the delta-frequency component of the movement speed (FIGS. 6I-6K).

During stimulation at both 10 Hz and 145 Hz, the nesting of LFP beta and gamma within the delta-rhythmic movement speed trace remained evident (FIGS. 6J-6K). Thus, delta-rhythmic movement organizes the timing of LFP beta and gamma regardless of sensory stimulation. Interestingly, 10 Hz stimulation, but not 145 Hz, selectively increased the coupling between movement delta phase and LFP beta power, but not gamma power (FIGS. 6J, 6K). Together, these results demonstrate that while audiovisual stimulation at both 10 Hz and 145 Hz promote movement, only 10 Hz stimulation enhances the delta rhythmicity of stepping (FIG. 6C, 6E), which is accompanied by strengthened delta-frequency phase locking between LFP and movement and increased coupling between LFP beta power and movement delta phase (FIG. 6J). These results highlight beta-frequency specific sensory modulation of striatal involvement in coordinating stepping on a moment-to-moment basis through improved coupling of striatal neural activity and movement.

### Example 4—Population Striatal LFP Dynamics were Better Entrained by Sensory Stimulation at 10 Hz than 145 Hz

In the next series of experimental examples, beta-frequency sensory stimulation on mice was evaluated. Audiovisual stimulation at 10 Hz or 145 Hz was delivered to mice voluntarily locomoting, simultaneously recording stepping movement, striatal cellular calcium dynamics and population local field potentials (LFPs). It was found that sensory stimulation promoted locomotion and desynchronized striatal network. However, only 10 Hz stimulation, but not 145 Hz, effectively entrained striatal LFPs, reduced movement transitions, enhanced stepping rhythmicity, and strengthened the coupling between stepping and striatal LFP delta and beta oscillations. These results highlight the therapeutic potential of non-invasive beta-frequency sensory stimulation for improving gait by strengthening motor processing of stepping movement.

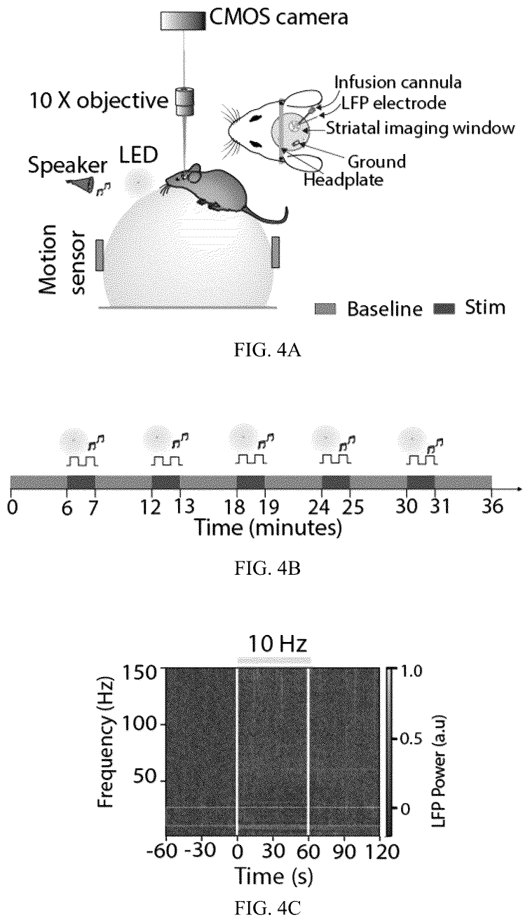

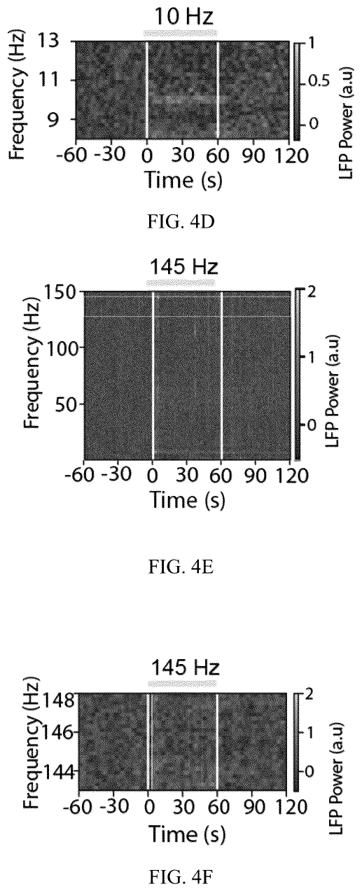

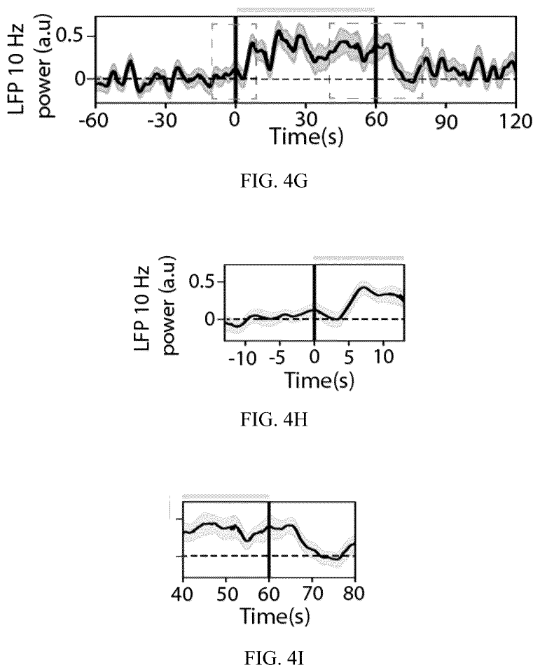

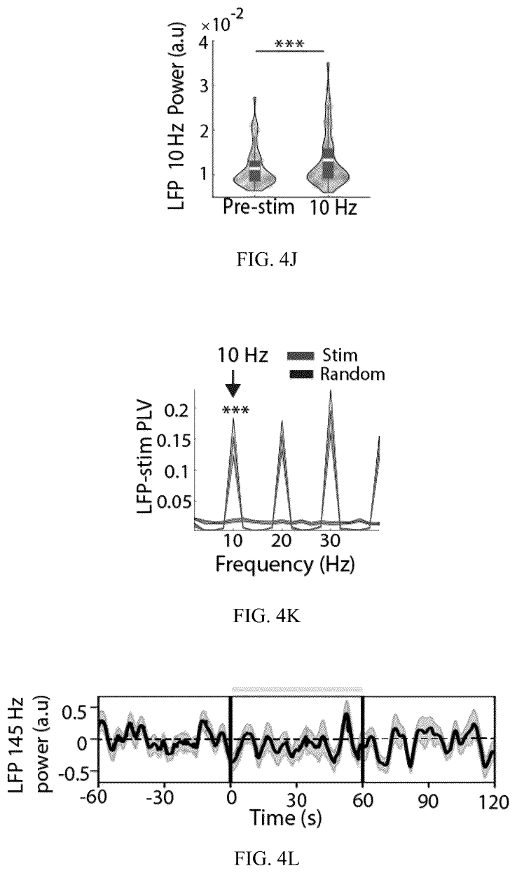

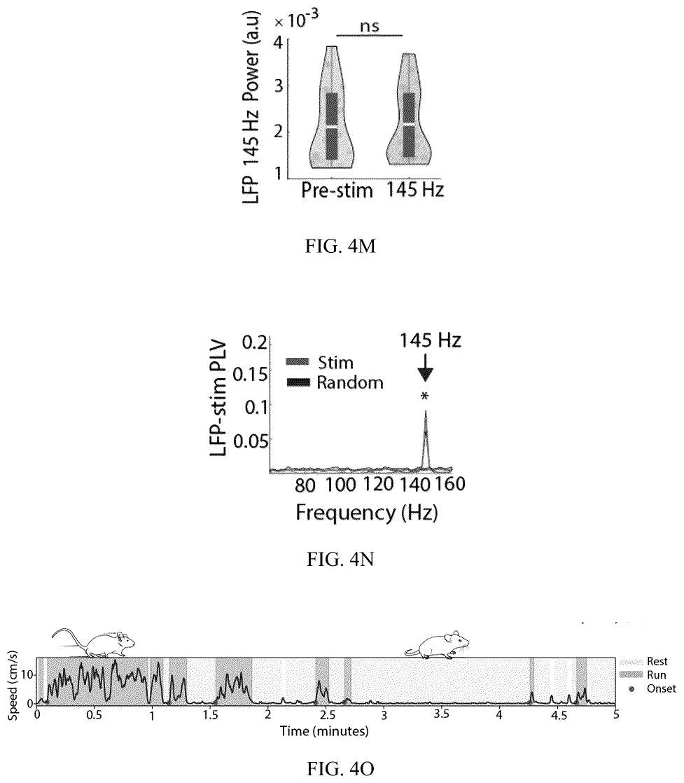

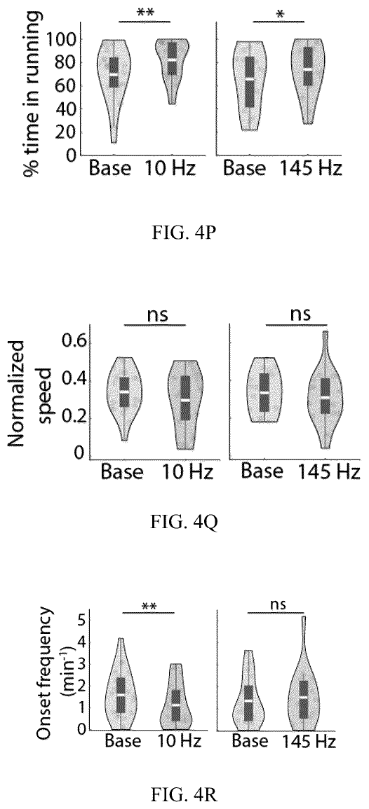

FIGS. 4A-4R illustrate features of this experimental example. FIG. 4A illustrates the experimental setup with a head-fixed mouse under a custom microscope voluntarily locomoting on a spherical treadmill. FIG. 4B illustrates the experimental timeline. FIG. 4C shows a normalized LFP power spectrum aligned to stimulation onset during 10 Hz stimulation. LFP power was Z-score to the mean pre-stimulation period. FIG. 4D shows a detailed view around 10 Hz as highlighted by the box in FIG. 4C. In FIGS. 4E and 4F, similar to FIGS. 4C and 4D, but during 145 Hz stimulation. In FIG. 4G, the mean 10 Hz LFP power aligned to stimulation onset across all 10 Hz stimulation sessions. In FIGS. 4H and 4I, a detailed view is shown around 10 Hz stimulation onset (FIG. 4H) and offset (FIG. 4I). In FIG. 4J, 10 Hz power during 10 Hz stimulation vs. pre-stimulation period (Wilcoxon signed rank test, p=8.9e⁻⁵, n=65 trials). FIG. 4K shows mean phase-locking value (PLV) between 10 Hz stimulation pulse trains and LFP across frequencies during stimulation (red) vs. random shuffles (black). PLV at 10 Hz is significantly greater during stimulation than random shuffles (Wilcoxon signed rank test, p=2.4e 4, n=24 trials). The shaded area is standard error of mean. FIG. 4L shows the mean 145 Hz LFP power aligned to stimulation onset across all 145 Hz stimulation sessions. FIG. 4M shows 145 Hz power during 145 Hz stimulation vs. pre-stimulation period (Wilcoxon signed rank test, p=0.44, n=24 trials). In FIG. 4N, the mean PLV of 145 Hz stimulation pulse train to LFP during stimulation (purple) vs. random shuffles (black) (Wilcoxon signed rank test, p=0.016). FIG. 4O shows an example locomotion speed recording, showing running bouts (dark blue shading), resting bouts (light blue shading), and onset transitions (red stars). FIG. 4P shows the percentage of time mice spent on running during 10 Hz or 145 Hz stimulation vs. baseline. Stimulation increased running time (Wilcoxon signed rank test, 10 Hz: p=0.0014, n=23 sessions; 145 Hz: p=0.022, n=17 sessions). FIG. 4Q shows speed during 10 Hz or 145 Hz stimulation relative to baseline. There was no difference (Wilcoxon signed rank test, 10 Hz: p=0.19, n=23 sessions; 145 Hz: p=0.98, n=17 sessions). FIG. 4R shows onset transition frequencies during 10 Hz and 145 Hz stimulation relative to baseline. Onset transition is significantly reduced for 10 Hz, but not 145 Hz stimulation (Wilcoxon signed rank test, 10 Hz: p=0.004, n=23 sessions; 145 Hz: p=0.523, n=17 sessions). Quantifications are visualized as violin plots with the outer shape representing the data kernel density, and a box plot (box: interquartile range, whiskers: 1.5× interquartile range, white line: mean). *p<0.05, **p<0.01, ***p<0.001.

To examine how non-invasive sensory stimulation at beta frequencies influences basal ganglia circuitry and locomotion, calcium imaging was performed from individual striatal neurons in mice freely navigating on a spherical treadmill (FIG. 4A). Briefly, striatal neurons were transduced with the genetically encoded calcium indicator GCaMP7f through intracranial infusion of AAV-syn-GCaMP7f (n=5 mice) or AAV-syn-SomaGCaMP7f (n=4 mice). GCaMP7f expressing neurons were then recorded using a custom microscope through an imaging window positioned above the striatum after gentle removal of the overlaying cortical tissue in one hemisphere (FIG. 4A). We also recorded conventional LFPs through a nearby electrode coupled to the imaging window, about 200 μm away from the imaging site (FIG. 4A). During each experiment, mice were presented with five one-minute-long audiovisual stimulation every five minutes, resulting in a total recording of 36 minutes per session (FIG. 4B). The audiovisual stimulation consisted of concomitant auditory clicks (˜77 dB, 50% duty cycle) and visual flashes (50% duty cycle) pulsed at 10 Hz or 145 Hz, in a dimly lit room with ˜72 dB constant background noise. We chose 10 Hz because it is readily supported by basal ganglia circuits, and thus likely entrains striatal circuits. In contrast, 145 Hz is generally thought to be too fast to mediate significant entrainment effect.

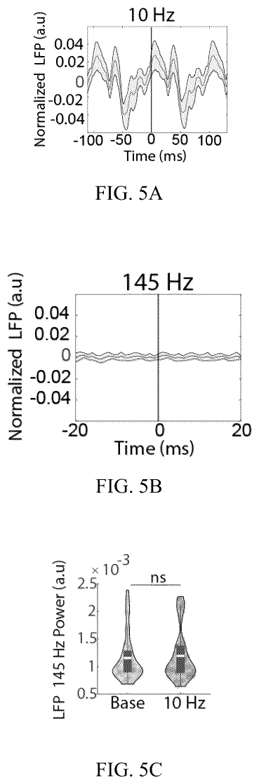

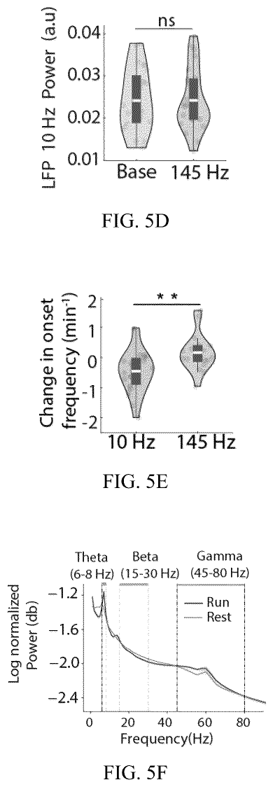

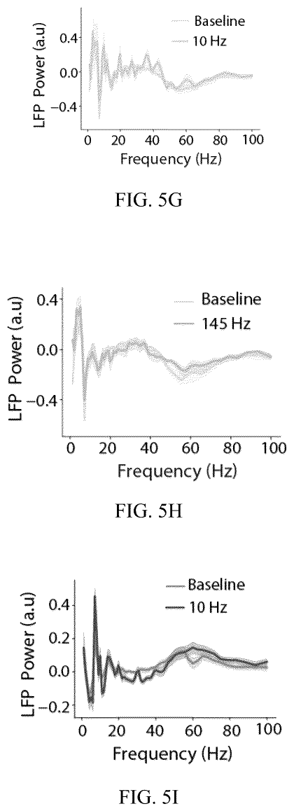

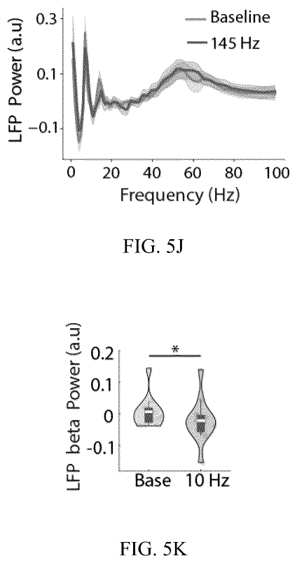

First, the sensory entrainment of LFP was evaluated by comparing LFP spectral power during stimulation versus the one-minute pre-stimulation period. As expected, 10 Hz stimulation significantly increased 10 Hz LFP spectral power during stimulation (FIGS. 4C, 4D, 4G, and 4J). Further, the population LFP power increase at 10 Hz lagged the stimulation onset by 5.5 sec (FIGS. 4G-4H) and remained elevated for 8.5 sec after stimulation offset (FIGS. 4G and 4I). Finally, 10 Hz stimulation exhibited significant phase locking to the 10 Hz component of the LFP signals, further confirming the entrainment effect (FIGS. 5A and 5K).

In contrast, 145 Hz stimulation did not alter the 145 Hz LFP spectral power (FIGS. 4E-4F, 4L-4M). Interestingly, we detected a weak but nonetheless significant phase locking between 145 Hz stimulation and the 145 Hz component of LFP signals, though the phase locking strength was an order of magnitude weaker than that observed with 10 Hz stimulation (FIGS. 5B, 5N). Moreover, 10 Hz stimulation did not alter 145 Hz LFP power (FIG. 5C), and 145 Hz stimulation did not alter 10 Hz LFP power (FIG. 5D), suggesting that the observed entrained effects were specific to the frequencies delivered via audiovisual stimulation. Together, these results demonstrate that audiovisual stimulation at 10 Hz produces prominent entrainment of the striatal network, whereas stimulation at 145 Hz mediates a much weaker effect.

FIGS. 5A-5K illustrate supplementary data for this experimental example. In particular, FIGS. 5A-5B illustrate population LFPs aligned to 10 Hz (FIG. 5A) and 145 Hz (FIG. 5B) stimulation pulse onset. Line: mean; shaded region: standard error of the mean. FIG. 5C shows LFP 145 Hz power during the 1-minute period pre-stimulation (base) and post-stimulation at 10 Hz (Wilcoxon signed rank test, 10 Hz: p=0.12, n=65 trials). FIG. 5D shows LFP 10 Hz power during the 1-minute period pre-stimulation (base) versus post-stimulation at 145 Hz stimulation (Wilcoxon signed rank test, p=0.75, n=24 trials). FIG. 5E shows difference in movement onset transition frequencies during stimulation vs baseline (stimulation-baseline) at 10 Hz and 145 Hz (Mann Whitney U test, p=0.002, 10 Hz: n=23 sessions, 145 Hz: n=17 sessions). FIG. 5F shows normalized LFP power spectrum density (PSD) during running (dark blue) and resting (light blue) in baseline periods across all sessions. Shaded region indicates standard error of the mean. FIG. 5G shows normalized LFP PSD during resting in baseline (blue) and 10 Hz stimulation (red/purple). FIG. 5H is the same as FIG. 5G, but for 145 Hz stimulation. There were no significant differences across conditions for theta, beta and gamma LFP power. FIG. 5I is the same as FIG. 5G, but during running in baseline versus 10 Hz stimulation. FIG. 5J is the same as FIG. 5I but for 145 Hz stimulation. FIG. 5K shows the change in LFP beta power during stimulation compared to baseline (stimulation—baseline) when mice were running (Wilcoxon signed rank test, p=0.034). Quantifications are visualized as violin plots with the outer shape representing the data kernel density and a box plot (box: interquartile range, whiskers: 1.5× interquartile range, white line: mean). *p<0.05, **p<0.01, ***p<0.001

### Example 5—Sensory Stimulation at Either 10 Hz or 145 Hz Increased the Time Mice Spent on Running, but Only 10 Hz Suppressed Movement Onset Transitions

We next examined the influence of sensory stimulation on movement by comparing locomotion features during stimulation versus baseline, defined as the period excluding the stimulation periods and the minute after to avoid any residual stimulation effects (FIG. 4O). We found that sensory stimulation at both frequencies increased the fraction of time mice spent on running (FIG. 4P), without changing the mean velocity during running (FIG. 4Q). Interestingly, 10 Hz stimulation, but not 145 Hz stimulation, led to a significant reduction in movement onset transitions (FIG. 4R, FIG. 5E), consistent with the general theory that beta rhythms contribute to maintaining the status quo.

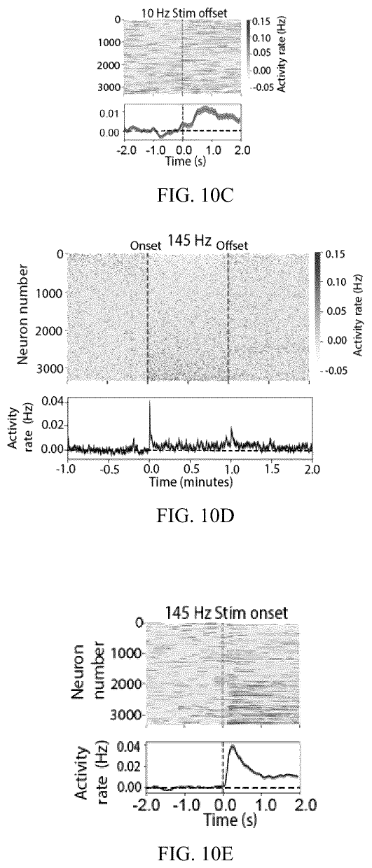

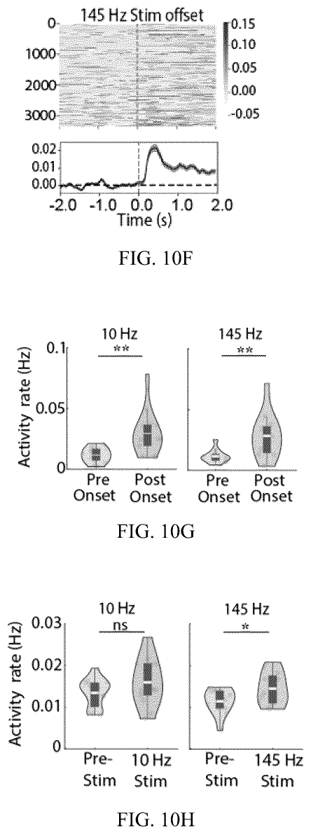

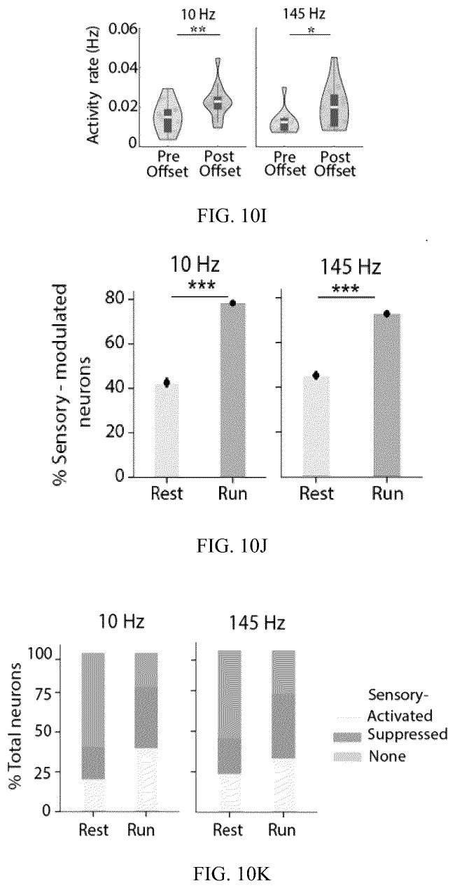

Striatal LFPs are known to be modulated by locomotion. During baseline without stimulation, we found that LFP theta (6-8 Hz) and gamma (45-80 Hz) power increased during movement, whereas beta (15-30 Hz) power decreased (FIG. 5F), consistent with our previous findings in mice under similar behavioral conditions. We picked the higher end (15-30 Hz) of the beta range to avoid the harmonics of theta and the direct 10 Hz entrainment effect. While sensory stimulation at either 10 Hz or 145 Hz did not change the LFP power at theta, beta, and gamma frequencies during rest (FIGS. 5G-5H), 10 Hz stimulation further weakened beta power during running relative to the baseline running condition (FIG. 5I, FIG. 5K). Thus, even though sensory stimulation at both frequencies promoted movement, only 10 Hz stimulation, but not 145 Hz, suppressed movement onset transitions. These results along with the differential effect of the two stimulation frequencies on entraining striatal LFPs highlight that beta-frequency sensory stimulation is effective at reducing movement flexibility, likely through entrainment of locomotor circuits.

### Example 6-10 Hz Stimulation, but not 145 Hz, Enhances Stepping Rhythmicity and Improves the Coupling Between Stepping and LFP Beta

As locomotion in mice and humans occurs in the delta frequency range, we first calculated the spectral power of the treadmill speed that tracked animal's stepping cycles. Consistent with our previous observations under similar locomotion conditions, mice stepping occurred at delta frequencies with a peak around 3-4 Hz (FIGS. 6A-6B). Upon 10 Hz stimulation, animal's movement became more rhythmic around delta frequencies (3-4 Hz), showing a significant increase in the relative delta power normalized across frequencies (FIG. 6C, 6E. In contrast, 145 Hz stimulation did not alter movement delta rhythmicity (FIGS. 6D-6E).

After detecting a change in stepping rhythmicity during 10 Hz stimulation, we next examined how sensory stimulation influences the temporal relationship between striatal population dynamics and stepping by computing the phase-locking value (PLV) between LFPs and movement speed. During running bouts, in the absence of sensory stimulation, there was a significant phase locking between LFPs and movement speed at delta frequencies (FIGS. 6F-6H), consistent with our previous findings. Interestingly, 10 Hz stimulation, but not 145 Hz stimulation, further promoted LFP-movement speed phase locking at delta frequencies during running (FIGS. 6F-6H).

Our previous study also showed that striatal LFP beta and gamma power are nested within the delta-rhythmic stepping cycles, peaking at a similar phase as spikes in delta-rhythmic striatal neurons. To understand how sensory stimulation influences the relationship between higher frequency components of the LFP power and stepping movement, we filtered the movement speed trace in the broader delta frequency range (2-4 Hz) and computed the mean LFP spectrogram around all movement delta-peaks. Consistent with our previous observations⁸, in the absence of stimulation, LFP beta and gamma power (40-100 Hz) were modulated by the delta-frequency component of the movement speed (FIGS. 6I-6K).

During stimulation at both 10 Hz and 145 Hz, the nesting of LFP beta and gamma within the delta-rhythmic movement speed trace remained evident (FIGS. 6J-76K). Thus, delta-rhythmic movement organizes the timing of LFP beta and gamma regardless of sensory stimulation. Interestingly, 10 Hz stimulation, but not 145 Hz, selectively increased the coupling between movement delta phase and LFP beta power, but not gamma power (FIG. 6J, FIG. 6K). Together, these results demonstrate that while audiovisual stimulation at both 10 Hz and 145 Hz promote movement (FIG. 4P), only 10 Hz stimulation enhances the delta rhythmicity of stepping (FIG. 6C, FIG. 6E), which is accompanied by strengthened delta-frequency phase locking between LFP and movement and increased coupling between LFP beta power and movement delta phase (FIG. 2J). These results highlight beta-frequency specific sensory modulation of striatal involvement in coordinating stepping on a moment-to-moment basis through improved coupling of striatal neural activity and movement.

FIG. 6A shows example movement speed trace (top) and corresponding power spectrogram (bottom) showing the delta rhythms. FIG. 6B shows Power spectral density (PSD) of movement speed during running normalized to resting across all recording periods. Black line corresponds to the mean across all recording sessions, and shading, the standard error of the mean (n=28 recording sessions). FIG. 6C is similar to FIG. 6B, but for 10 Hz stimulation sessions only, with Baseline in blue and 10 Hz stimulation in red. FIG. 6D is the same as FIG. 6C, but for 145 Hz stimulation sessions. FIG. 6E shows quantification of speed delta (3-4 Hz) PSD across all running bouts during baseline vs. 10 Hz or 145 Hz stimulation (Wilcoxon signed rank test, 10 Hz, p=0.01, n=14 sessions; 145 Hz: p=0.17, n=14 sessions). FIG. 6F shows LFP-movement speed PLV across frequencies during baseline (blue) vs. 10 Hz stimulation (red).

FIG. 6G is the same as FIG. 6F, but for 145 Hz stimulation. FIG. 6H shows LFP—movement speed delta PLV during baseline vs. 10 Hz or 145 stimulation (Wilcoxon signed rank test, 10 Hz: baseline vs. shuffle, p=2.4e⁻⁴, stimulation vs. shuffle: p=0.001, baseline vs. stimulation: p=0.008, n=13; 145 Hz: baseline vs. shuffle: p=0.002, stimulation vs. shuffle: p=0.006, baseline vs. stimulation: p=0.74, n=13). FIG. 6I shows a heatmap of the relative LFP power aligned to the peak of the delta-frequency component of movement speed. FIG. 6J shows cross frequency coupling (CFC) between movement speed delta and beta-filtered LFP (15-30 Hz) during baseline vs. 10 Hz or 145 Hz stimulation (Wilcoxon signed rank test, 10 Hz: baseline: p=2.4e⁻⁴, 10 Hz: p=2.4e⁻⁴, baseline vs 10 Hz: p=0.03; 145 Hz: baseline: p=2.4e⁻⁴, 145 Hz: p=2.4e⁻⁴, baseline vs 145 Hz: p=0.24). FIG. 6K shows CFC between movement delta and gamma-filtered LFP during baseline vs. 10 Hz or 145 Hz stimulation (Wilcon signed rank test, 10 Hz: baseline: p=2.4e⁻⁴, 10 Hz: p=2.4e⁻⁴, baseline vs. 10 Hz: p=0.45; 145 Hz: baseline: p=2.4e⁻⁴, 145 Hz: p=2.4e⁻⁴, baseline vs. 145 Hz: p=0.73). Lines represent the mean across recording sessions and shaded regions are the standard error of the mean. Quantifications are visualized as violin plots with the outer shape representing the data kernel density and a box plot (box: interquartile range, whiskers: 1.5× interquartile range, white line: mean). *p<0.05, **p<0.01, ***p<0.001.

### Example 7—Cellular Calcium Activity Precedes Locomotion

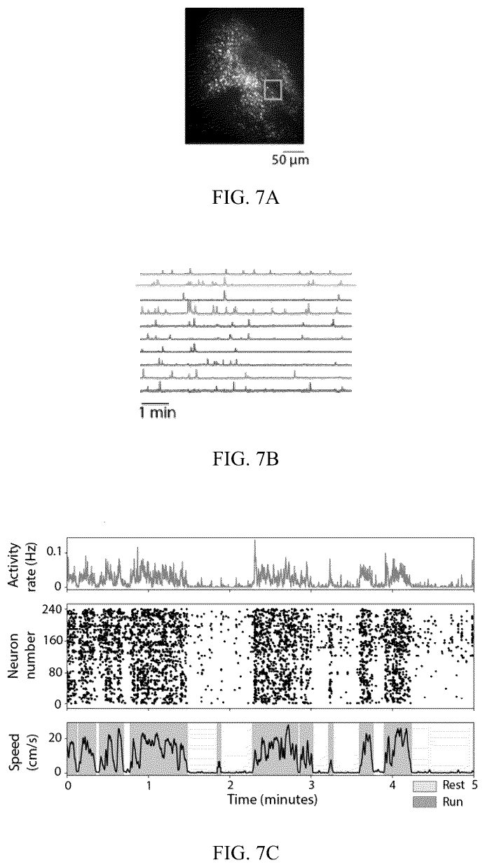

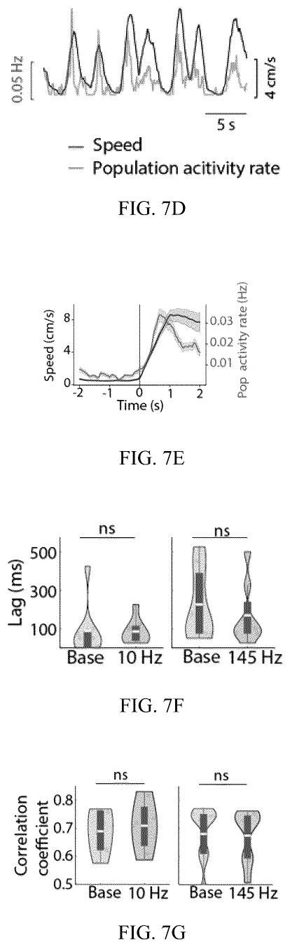

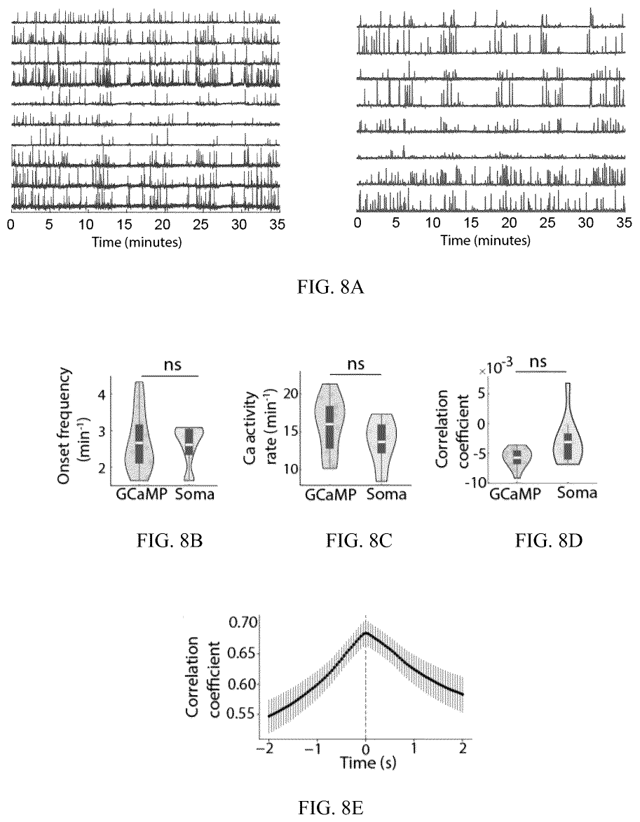

To examine how sensory stimulation modulates striatal encoding of locomotion, we performed single cell calcium imaging from hundreds of striatal neurons simultaneously using GCaMP7f that is better at capturing spiking than older generations of GCaMPs (FIGS. 7A-7B). While intracellular calcium concentration fluctuates due to various biochemical and biophysical activities, the rising phase of the transiently occurring calcium events is generally associated with elevated spiking probabilities, whereas the falling phase is influenced by the slow kinetics of calcium ion dissociation from GCaMP7f and calcium clearance from the cytosol. Thus, we identified calcium events in the GCaMP7f fluorescence traces and binarized the event rising phase as ones and everywhere else as zeros to capture periods with heightened neuronal activity (FIG. 8A). During baseline without stimulation, we detected 2.45±1.58 calcium events/min (mean±standard deviation, n=6409 neurons, all experimental sessions), consistent with previous calcium imaging studies of striatal neurons using GCaMP7, but higher than that using the less sensitive GCaMP6f. Using the rising phase of each calcium event, we obtained an activity rate of 1.26±0.72%, to approximate the percentage of time neurons have heightened spiking probability. As with our previous study⁵³, we detected no difference between GCaMP7f and Soma-GCaMP7f in terms of calcium event rate, activity rate, or pairwise Pearson correlation coefficients (FIGS. 8B-8D). Thus, all subsequent analyses were performed by combining the two mice groups.

As many striatal neurons are known to increase their activity during movement, we first determined the fraction of neurons that were modulated by locomotion under our experimental conditions. Briefly, we computed the difference in activity rate between running and resting bouts and then compared it to a shuffled distribution computed from two randomly chosen baseline periods. Since calcium activity rate is generally low, we identified movement-responsive neurons as those with a significant increase in activity rate (>97.5th percentile in shuffled distribution) during running compared to resting bouts. During the baseline period without sensory stimulation, we found 70.4% neurons were activated by movement (FIG. 7C, 4289/6096 neurons from 24 sessions).

We also noticed that the activity of movement-responsive neurons preceded the rise in speed (FIGS. 7D-7E). To quantify this temporal relationship, we computed the cross correlation between the population activity of movement-responsive neurons and locomotion speed with different temporal lags and identified the time lag when the peak correlation occurred. Across baseline sessions without stimulation, the peak correlation was 0.68±0.02 (mean±standard error, n=16 sessions, FIG. 8E), with neuronal responses preceding locomotion by 156±42.13 ms (mean±standard error, n=16 sessions; range: 50-525 ms; median: 75 ms). This result is in general agreement with prior electrophysiology findings showing striatal neuron activation before movement. Further, audiovisual stimulation did not alter the time lags (FIG. 7F) or the peak correlation coefficient (FIG. 7G). Thus, even though sensory stimulation heightened locomotion bouts (FIG. 4P), it did not alter the temporal relationship of striatal activity and locomotion.

FIG. 7A shows a maximum-minimum GCaMP7 fluorescence intensity image from an example recording session. FIG. 7B shows example fluorescence traces from neurons in the yellow box in FIG. 7A. FIG. 7C shows an example recording showing mean calcium activity rate across movement-responsive neurons (top), the raster plot displaying calcium activity rate across individual neurons (middle), and the corresponding movement speed (bottom), with resting (light blue) and running (dark blue) bouts highlighted. FIG. 7D shows instantaneous population activity rate (green) across movement-responsive neurons in one example recording session and corresponding movement speed (black). FIG. 7E shows average population activity rate of movement-responsive neurons (green) in the same example session as in FIG. 7D aligned to movement onsets (average speed trace in black). FIG. 7F shows the time lags between activity rate and movement speed during baseline and 10 Hz or 145 Hz stimulation (Wilcoxon signed rank test, 10 Hz: p=0.46, n=8; 145 Hz, p=0.16, n=8). FIG. 7G shows the peak correlation coefficients during baseline vs. 10 Hz or 145 Hz stimulation (Wilcoxon signed rank test, 10 Hz, p=0.5, n=8; 145 Hz, p=0.58, n=8). Quantifications are visualized as violin plots with the outer shape representing the data kernel density and a box plot (box: interquartile range, whiskers: 1.5× interquartile range, white line: mean). *p<0.05, **p<0.01, ***p<0.001.

FIG. 8A shows an example GCaMP7f traces from representative neurons during a typical recording session. The rising phases of identified events were marked with red. FIG. 8B shows the mean calcium event onset frequencies across all GCaMP7f baseline recording sessions without any stimulation vs SomaGCaMP7f baseline recording sessions. Each individual dot corresponds to the mean event frequencies across all neurons in a session. There was no difference (GCaMP7f: 2.67±0.79 events/min, n=15 sessions, somaGcamp7: 2.61±0.51 events/min, n=11, Independent t-test, p=0.86). FIG. 8C shows the mean calcium event activity rate across all GCaMP7f baseline recording sessions versus SomaGcaMP7f baseline recording sessions. There was no difference (GCaMP7f: 16±3.41/min, n=15, SomaGcaMP7f: 13.67±2.87/min, n=11, Independent t-test, p=0.08). FIG. 8D shows the median correlation coefficients across all GCaMP7f baseline recording sessions versus SomaGcaMP7f baseline recording sessions (Wilcoxon rank sum test, p=0.052, n=15 GCaMP7f sessions and n=11 SomaGCaMP7f sessions). FIG. 8E shows the population correlation coefficient across time lags, aligned to peak correlation coefficient across all recording sessions. Quantifications are visualized as violin plots with the outer shape representing the data kernel density and a box plot (box: interquartile range, whiskers: 1.5× interquartile range, white line: mean). *p<0.05, **p<0.01, ***p<0.001.

### Example 8—Cellular Neuronal Activity Tracks Delta-Rhythmic Movement and Sensory Stimulation does not Alter this Temporal Relationship

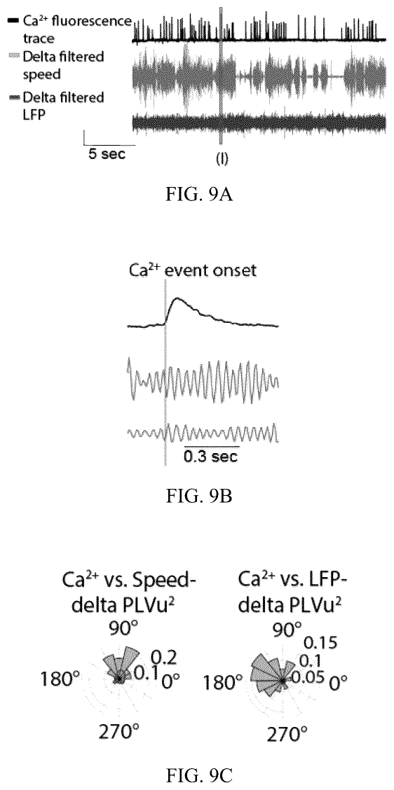

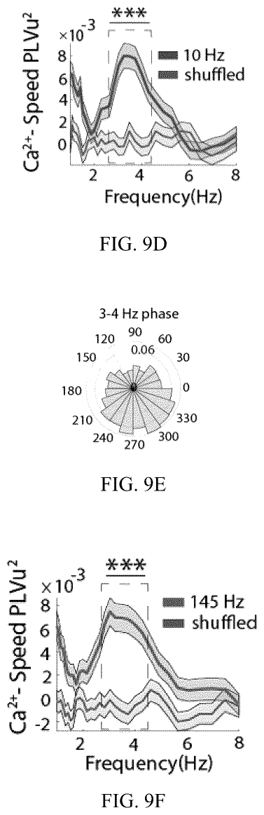

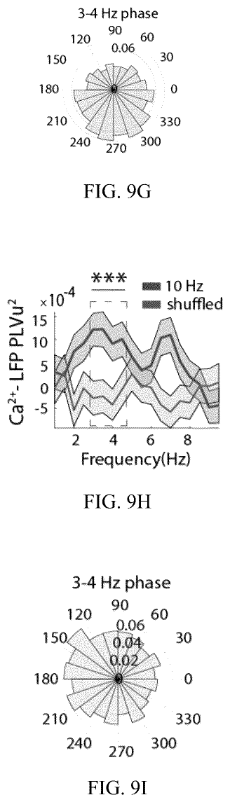

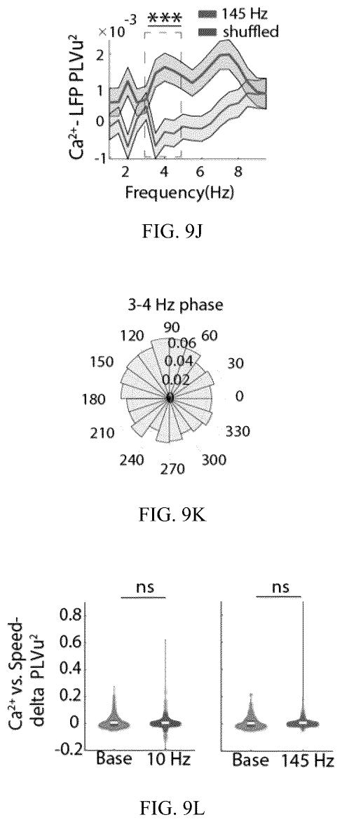

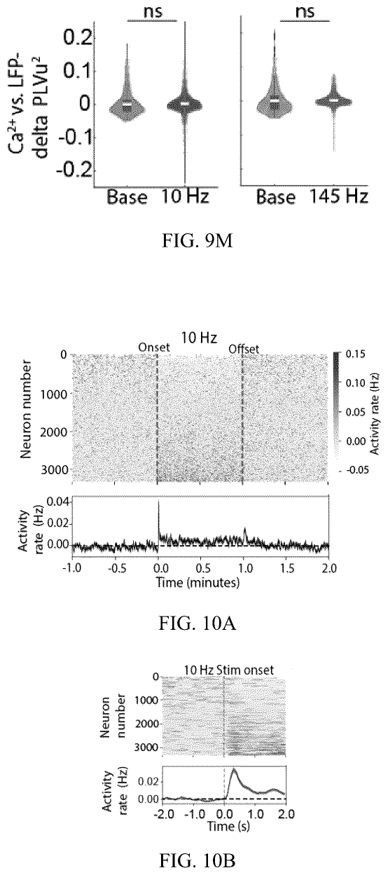

After observing a temporal relationship between population LFP delta and movement stepping cycles, we further evaluated the relationship between individual neurons and stepping. Even though calcium dynamics is slow, we found that calcium event onsets exhibited significant phase locking to delta-rhythmic movement speed (FIGS. 9A-9G). Audiovisual stimulation did not change the phase locking strength (FIG. 9I). Furthermore, event onsets were also phase locked to the delta component of the LFP trace (FIGS. 9A-9C, 9H-9K), and stimulation did not alter the phase locking strength (FIG. 9M). The prominent phase locking of calcium events to stepping movement and LFP delta oscillations confirms that individual striatal neurons are coordinated with step-by-step movement, as demonstrated in our previous study, and sensory stimulation does not alter the temporal relationship between striatal cellular dynamics and stepping movement.

FIG. 9A shows an example neuron's fluorescence trace with corresponding delta-frequency filtered movement speed trace and delta-frequency filtered LFP trace. FIG. 9B shows a zoom-in view around a calcium event onset indicated by the green line in FIG. 9A. FIG. 9C shows polar histograms of the distribution of the preferred phase of all calcium event onsets relative to delta-filtered speed (left) and delta-filtered LFP (right) for the example neuron in FIG. 9A. FIG. 9D shows PLVu² of calcium event onsets across all neurons to movement speed during running (red) and random shuffles (gray) throughout the entire recording session with 10 Hz stimulation. FIG. 9E shows the polar histogram of neurons' preferred calcium event phase (mean angle) to delta-filtered speed (paired t-test, 10 Hz: p=1.5e⁻⁹, n=2510). FIGS. 9F-9G are the same as FIGS. 9D-9E, but for sessions with 145 Hz stimulation (Paired t-test, p=2.8e⁻⁸, n=2669). FIG. 9H shows the PLVu² of calcium event onsets to LFP across frequencies during running (red) and random shuffles (gray) throughout the entire recording session with 10 Hz stimulation, demonstrating significant phase locking at delta frequencies (paired t-test, p=6.1e⁵, n=2510). FIG. 9I shows the polar histogram of the phase of calcium event onsets to LFP delta oscillations across all neurons in sessions with 10 Hz stimulation. FIGS. 9J-9K are the same as FIGS. 9H, 9I, but for sessions with 145 Hz stimulation (paired t-test, p=0.0026, n=2669). FIG. 9L shows quantification of Ca²⁺— speed delta PLVu² during baseline vs. stimulation across 10 Hz and 145 Hz sessions (paired t-test, 10 Hz: p=0.37, n=905; 145 Hz: p=0.28, n=684). FIG. 9M shows a quantification of PLVu² between calcium event onsets and delta-component of LFP in baseline vs. 10 Hz or 145 Hz stimulation (paired t-test, 10 Hz: p=0.43, n=885; 145 Hz: t-test, p=0.32, n=676). Lines represent the mean and the shaded regions represent the standard error of the mean. Quantifications are visualized as violin plots with the outer shape representing the data kernel density and a box plot (box: interquartile range, whiskers: 1.5× interquartile range, white line: mean). *p<0.05, **p<0.01, ***p<0.001.

### Example 9—Sensory Stimulation at 10 Hz and 145 Hz Modulated a Greater and Yet Balanced Fraction of Activated Versus Suppressed Neurons During Movement

Aligning the response of individual neurons to audiovisual stimulation onset, we found that stimulation evoked responses are most prominent at the stimulation onset, with some neurons exhibiting increased calcium event rates and others decreased rates (FIGS. 10A, 10D).

As a population, evoked responses showed a sharp increase at stimulation onset, peaking around 0.3 s after onset, and then settling into a sustained increase after about 1 second, for both stimulation frequencies (FIGS. 10B, 10E). While the transient population increase within one second of stimulation onset was significant for both frequencies (FIG. 10G), the sustained increase was only significant for 145 Hz stimulation, but not for 10 Hz (FIG. 10H). At stimulation offset, we also detected a significant rebound, peaking around 0.5 s after the offset (FIGS. 10C, 10F, 10I).

Next, we determined whether each neuron was modulated, either positively or negatively, by sensory stimulation by comparing the activity rate during resting or running separately using the shuffling procedure as that for determining movement-responsive neurons (details in Methods). During resting, about 44% neurons were modulated by sensory stimulation (FIG. 10J, Table 1), and during running, a significantly larger fraction (˜75.5%) were modulated (FIG. 10J, Table 1-2, Fisher's test, 10 Hz: p=1.17e⁻¹⁴²; 145 Hz: p=5.24e⁻⁸⁹). Intriguingly, the proportions of positively and negatively modulated neurons were similar during both resting and running for both stimulation frequencies (FIG. 10K, Table 1).

TABLE 1
Sensory-responsive neurons during resting
and running (related to FIG. 10K)

| | | Sensory- | Number | | |
|---|---|---|---|---|---|
| | | responsive | over | | |
| | | during | total neurons | | |
| | | resting | recorded | Percentage | n |
| 10 | Hz | Activated | 412/1813 | 22.72% | n = 7 sessions, |
| | | Suppressed | 360/1813 | 19.86% | 5 mice |
| | | Activated + | 772/1813 | 42.58% | |
| | | Suppressed | | | |
| 145 | Hz | Activated | 562/2292 | 24.52% | n = 6 sessions, |
| | | Suppressed | 482/2292 | 21.03% | 5 mice |
| | | Activated + | 1044/2292 | 45.55% | |
| | | Suppressed | | | |
| 10 | Hz | Activated | 1260/3155 | 39.94% | n = 13 sessions, |
| | | Suppressed | 1214/3155 | 38.48% | 9 mice |
| | | Activated + | 2474/3155 | 78.41% | |
| | | Suppressed | | | |
| 145 | Hz | Activated | 909/2739 | 33.19% | n = 13 sessions, |
| | | Suppressed | 1094/2739 | 39.94% | 9 mice |
| | | Activated + | 2003/2739 | 73.13% | |
| | | Suppressed | | | |

TABLE 2
Fisher's test (related to FIG. 10J)

| | Resting | Running |
|---|---|---|
| 10 Hz | | |
| Modulated | 712 (42.6%) | 2474 (78.4%) |
| Non-modulated | 1041 (57.4%) | 681 (21.6%) |
| 145 Hz | | |
| Modulated | 1044 (45.5%) | 2003 (73.1%) |
| Non-modulated | 1248 (54.5%) | 736 (26.9%) |

FIG. 10A shows a heatmap of the activity rate of all neurons before, during and after 10 Hz stimulation (top), and their population average (bottom). Red dotted lines correspond to stimulation onset and offset. FIGS. 10B-10C show zoom-in views around the 10 Hz stimulation onset (FIG. 10B) and offset (FIG. 10C). FIG. 10D is the same as FIG. 10A, but for 145 Hz stimulation. FIGS. 10E-10F show zoomed-in views around 145 Hz stimulation onset (FIG. 10E) and offset (FIG. 10F). FIG. 10G shows the quantification of transient activity rate during 1 second period pre- vs. post-onset of 10 Hz or 145 stimulation (Wilcoxon signed rank test, 10 Hz: p=0.003, n=14; 145 Hz, p=0.005, n=12). FIG. 10H shows the quantification of sustained activity rate during a minute pre- vs. post-onset of 10 Hz or 145 Hz stimulation (Wilcoxon signed rank test, 10 Hz: p=0.54, 145 Hz: p=0.016). FIG. 10I shows the quantification of the transient activity rate during 1 second pre- vs. post-offset of 10 Hz or 145 stimulation (Wilcoxon signed rank test, 10 Hz: p=0.001; 145 Hz: p=0.02). FIG. 10J shows the quantification of the fraction of modulated neurons during resting (light blue) vs. running (dark blue) for 10 Hz and 145 Hz sessions (Fisher's test: 10 Hz: p=1.17e⁻¹⁴²; 145 Hz: p=5.24e⁻⁸⁹). FIG. 10K shows a stacked bar plot visualization of the proportions of sensory-activated (yellow), sensory-suppressed (teal) and non-modulated neurons (gray) in each locomotor condition. Quantifications are visualized as violin plots with the outer shape representing the data kernel density and a box plot (box: interquartile range, whiskers: 1.5× interquartile range, white line: mean). *p<0.05, **p<0.01, ***p<0.001.

### Example 10—Sensory Stimulation Inhibited Movement-Responsive Neurons' Population Activity During Running

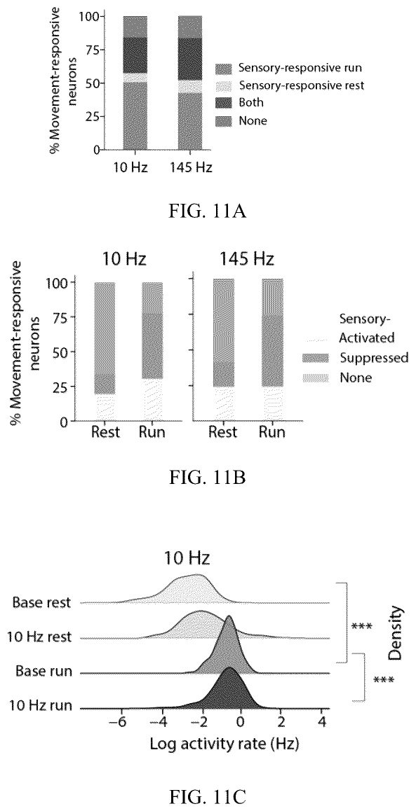

Further evaluation of movement-responsive neurons revealed that ˜84% were also responsive to sensory stimulation (FIG. 11A, Table 3), suggesting that many striatal neurons integrate sensory and motor information. We next characterized how sensory stimulation modulates movement-responsive neurons and found that more neurons were inhibited by sensory stimulation during running than during resting (FIG. 111B, FIG. 12A, Table 4-5, Fisher's test, 10 Hz: p=3.4e⁻⁶⁴; 145 Hz: p=5.6e⁻⁷⁸). Similarly, at the population level, sensory stimulation led to a robust suppression of movement-responsive neurons during running but not resting FIG. 11C-11D, FIG. 12B). Thus, sensory stimulation led to heterogenous changes of single neuron activities in a locomotion-state dependent manner. While the overall fraction of activated neurons was balanced by the fraction of inhibited neurons regardless of locomotion state, sensory stimulation robustly inhibited movement-related population dynamics during running.

TABLE 3
Overlap between sensory-responsive and movement-
responsive neurons (related to FIG. 11A)

| | | | Number over total | |
|---|---|---|---|---|
| | | Category | neurons recorded | Percentage |
| 10 | Hz | Sensory- | 75/1107 | 6.78% |
| | | responsive during | | |
| | | resting only | | |
| | | Sensory- | 557/1107 | 50.32% |
| | | responsive during | | |
| | | running only | | |
| | | Both | 299/1107 | 27.01% |
| | | None | 176/1107 | 15.9% |
| 145 | Hz | Sensory- | 135/1423 | 9.49% |
| | | responsive during | | |
| | | resting only | | |
| | | Sensory- | 601/1423 | 42.23% |
| | | responsive during | | |
| | | running only | | |
| | | Both | 452/1423 | 31.76% |
| | | None | 235/1423 | 16.52% |

TABLE 4
Movement-responsive neurons that are sensory-responsive
during resting vs. running (related to FIG. 11B)

| | | Sensory- | Number | | |
|---|---|---|---|---|---|
| | | responsive | over | | |
| | | during | total neurons | | |
| | | resting | recorded | Percentage | n |
| 10 | Hz | Activated | 215/1107 | 19.42% | n = 7 sessions, |
| | | Suppressed | 159/1107 | 14.36% | 5 mice |
| | | Activated + | 374/1107 | 33.79% | |
| | | Suppressed | | | |
| 145 | Hz | Activated | 340/1423 | 23.89% | n = 6 sessions, |
| | | Suppressed | 247/1423 | 17.36% | 5 mice |
| | | Activated + | 587/1423 | 41.25% | |
| | | Suppressed | | | |
| 10 | Hz | Activated | 336/1107 | 30.35% | n = 7 sessions, |
| | | Suppressed | 520/1107 | 46.97% | 5 mice |
| | | Activated + | 856/1107 | 77.33% | |
| | | Suppressed | | | |
| 145 | Hz | Activated | 341/1423 | 23.96% | n = 6 sessions, |
| | | Suppressed | 712/1423 | 50.04% | 5 mice |
| | | Activated + | 1053/1423 | 74% | |
| | | Suppressed | | | |

FIG. 11A shows a stacked bar plot illustration of the fraction of movement-responsive neurons that was responsive to 10 Hz (left) or 145 Hz (right) stimulation during resting (sensory resp. rest, light blue), during running (sensory resp. run, dark blue), during both resting and running (both, purple), and irresponsive (none, gray). FIG. 11B shows a stacked bar plot illustration of the fractions of movement responsive neurons that are activated by 10 Hz (left) and 145 Hz (right) stimulation (activated, yellow), suppressed (suppressed, teal) and non-modulated (none, gray), during resting vs. running. FIG. 11C shows a ridgeline plot illustration of population probability density of mean activity rate across movement-responsive neurons during resting or running, with vs. without 10 Hz stimulation (Mixed effect models, Baseline resting vs 10 Hz resting: pₐₚₚᵣₒₓ=1, Baseline running vs 10 Hz running: pₐₚₚᵣₒₓ=3.74e¹¹⁴, n=2189). FIG. 11D is the same as 12E, but during 145 Hz stimulation sessions (Baseline resting vs 145 Hz resting: pₐₚₚᵣₒₓ=1, Baseline running vs 145 Hz running: pₐₚₚᵣₒₓ=4.6e⁻¹⁵⁸, n=2041). *p<0.05, **p<0.01, ***p<0.001.

FIG. 12A shows the fraction of movement responsive neurons that were suppressed by sensory stimulation during resting (light blue) and running (dark blue) across 10 Hz and 145 Hz stimulation sessions (Fisher's test, 10 Hz: p=3.4e⁻⁶⁴, 145 Hz: p=5.6e⁷⁸). FIG. 12B shows the mean activity rate during baseline vs. stimulation at 10 Hz and 145 Hz (Mixed effect models, 10 Hz: pₐₚₚᵣₒₓ=3.7e⁻¹¹⁴, 145 Hz: pₐₚₚᵣₒₓ=4.6e⁻¹⁵⁸). Quantifications are visualized as violin plots with the outer shape representing the data kernel density and a box plot (box: interquartile range, whiskers: 1.5× interquartile range, white line: mean). *p<0.05, **p<0.01, ***p<0.001.

TABLE 5
Fisher's test (related to FIG. 12A)

| | Resting | Running |
|---|---|---|
| 10 Hz | | |
| Suppressed | 159 (14.4%) | 520 (47%) |
| Non-suppressed | 948 (85.6%) | 587 (53%) |
| 145 Hz | | |
| Suppressed | 247 (17.3%) | 712 (50%) |
| Non-suppressed | 1176 (82.7%) | 711 (50%) |

### Example 11—Both Locomotion and Sensory Stimulation Desynchronize Movement-Responsive Neuronal Networks

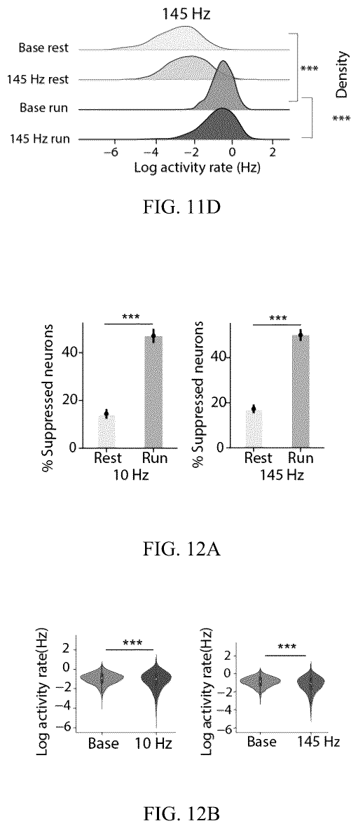

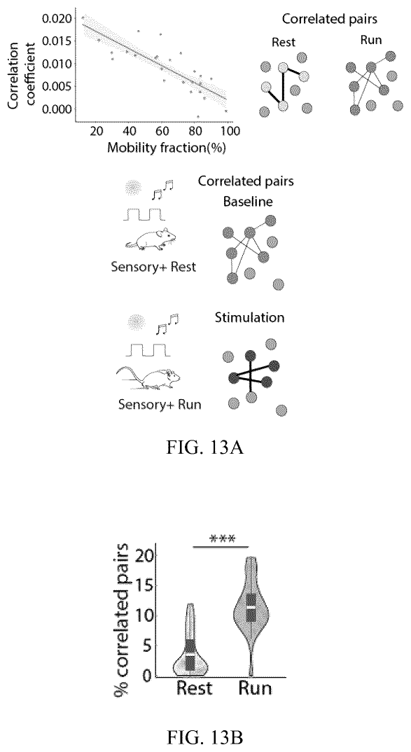

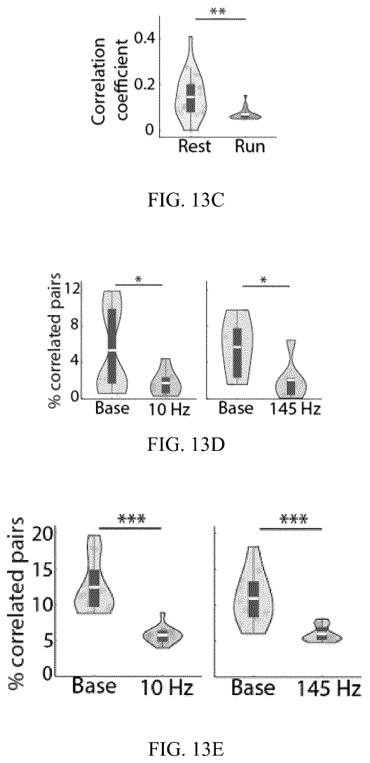

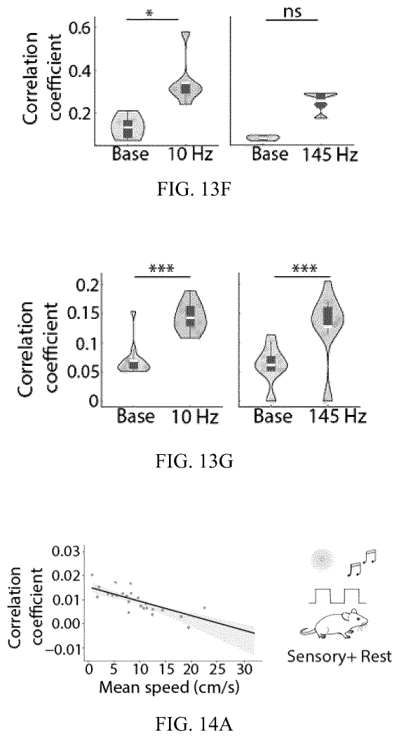

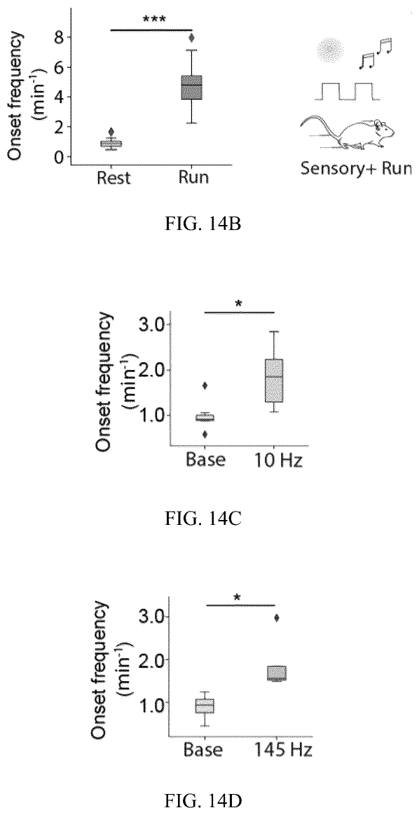

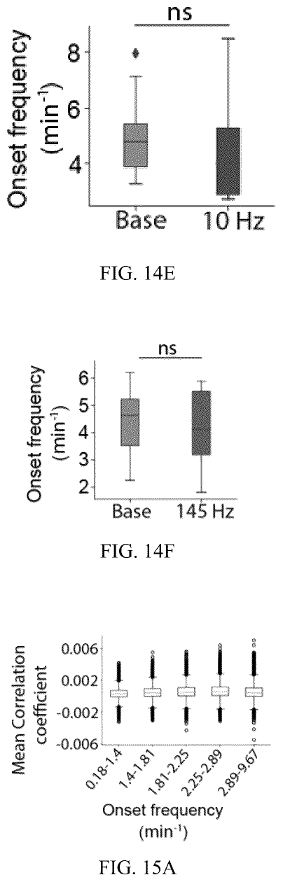

To further understand how sensory-evoked single neuron responses relate to network dynamics, we calculated pairwise Pearson correlations between movement-responsive neurons, as these neurons are most relevant to the locomotion behavior tested. We found that the overall correlation coefficients between these neurons across the entire recording sessions showed a negative linear relationship with the fraction of time the mice spent in running (FIG. 13A, R²=0.66, p=7.6e⁻⁷, n=25 sessions) and the average speed of the session (FIG. 14A), suggesting that locomotion generally desynchronizes the movement-responsive neural network, consistent with our previous study.

To further evaluate the impact of sensory stimulation and locomotion on functional connectivity, we identified neuron pairs that exhibit significant correlations given their corresponding activity rates. Briefly, we compared the correlation coefficient of a neuron pair to a shuffled distribution formed by assigning random lags between the neuron pair. If the observed correlation value was greater than 97.5^{th} percentile of the shuffled distribution (1000 shuffles), the neuron pair was deemed a ‘correlated pair’, otherwise a ‘random pair’. We found that the fraction of correlated pairs during running (run-relevant pairs) is significantly higher than during resting (rest-relevant pairs) (FIG. 13B). Since the activity rates of these movement-responsive neurons increased during running (FIG. 14B), we computed the normalized correlation coefficient of each neuron pair by subtracting the median correlation of the corresponding shuffles. Interestingly, the normalized correlation coefficients among run-relevant pairs were significantly weaker than the rest-relevant pairs (FIG. 13C), which cannot be attributed to the activity rate differences between the populations (FIGS. 14C-14F).

During sensory stimulation, the fraction of correlated pairs was significantly lower than baseline during both resting and running (FIG. 13D, FIG. 13E). Furthermore, stimulation enhanced the correlation strength of the correlated pairs compared to baseline, regardless of the movement state (FIG. 13F, FIG. 13G), which cannot be explained by the variation in activity rates (FIGS. 14C-14F). Together, these results demonstrate that locomotion desynchronizes movement-responsive neural networks by reducing connectivity strength, even though the fraction of correlated pairs increases during movement. Similarly, sensory stimulation also desynchronizes the striatal network, but by reducing the fraction of correlated pairs.

FIG. 13A shows median correlation coefficients across movement-responsive neurons vs. the percentage of time mice spent running (mobility fraction). There is a significant linear relationship (linear regression, R²=0.66, p=7.6e⁻⁷, n=25 sessions) with shaded regions indicating the 95% confidence interval. FIG. 13B shows percentage of correlated pairs during resting vs. running in the baseline period without stimulation (Wilcoxon signed rank test, p=2.3e⁻⁵, n=25). FIG. 13C shows correlation strength of the correlated pairs during resting vs. running in the baseline period without stimulation (Wilcoxon signed rank test, p=0.002, n=25). FIG. 13D shows percentage of correlated neuron pairs during baseline vs. 10 Hz (left) or 145 Hz (right) stimulation (Wilcoxon signed rank test, 10 Hz: p=0.016, n=7; 145 Hz: p=0.03, n=6). FIG. 13E is the same as FIG. 13D, but during running (Wilcoxon signed rank test, 10 Hz:1.2e⁻⁴, n=14; 145 Hz: 9.8e⁻⁴, n=11). FIG. 13F shows quantification of correlation strength across correlated pairs during resting during baseline vs. 10 Hz (left) or 145 Hz (right) stimulation (Wilcoxon signed rank test, 10 Hz: p=0.016, n=7; 145 Hz: p=0.06, n=6). FIG. 13G is the same as FIG. 13F, but during running (10 Hz: p=2.4e⁻⁴, n=14; 145 Hz: p=4.9e⁻⁴, n=11). Quantifications are visualized as violin plots with the outer shape representing the data kernel density and a box plot (box: interquartile range, whiskers: 1.5× interquartile range, white line: mean). *p<0.05, **p<0.01, ***p<0.001.

FIG. 14A shows median correlation coefficients across movement-responsive neurons vs. mean speed across the session. Each dot corresponds to a recording session. There is a significant linear relationship (linear regression, R²=0.62, p=2.8e⁻⁶, n=25 sessions) with shaded regions indicating the 95% confidence interval. FIG. 14B shows session-wise mean pairwise activity rate during resting vs. running in baseline periods without stimulation (Wilcoxon signed rank test, p=2.7e⁻⁵, n=25 sessions). FIG. 14C shows session-wise mean pairwise activity rate across correlated neuron pairs during resting in baseline and for 10 Hz stimulation (Wilcoxon signed rank test, p=0.018, n=7 sessions). FIG. 14D is the same as FIG. 14C, but for 145 Hz stimulation (Wilcoxon signed rank test, p=0.028, n=6). FIG. 14E is the same as FIG. 14C, but during running (Wilcoxon signed rank test, p=0.14). FIG. 14F is the same as FIG. 14E, but for 145 Hz stimulation (Wilcoxon signed rank test, p=0.07).

FIG. 15A shows mean correlation coefficients across shuffles versus calcium event onset rate (per min). FIG. 15B shows thresholds (97.5^{th} percentile) across shuffles versus calcium event onset rate. FIG. 15C is the same as FIG. 15A, but for median correlation coefficients across shuffles. FIG. 15D is the same as FIG. 15C, but for median correlation coefficients in the observed data.

TABLE 6
Summary of all results

| Category | Measure | Periods | 10 Hz | 145 Hz |
|---|---|---|---|---|
| LFP entrainment | Power at the stimulation frequency | Baseline vs. stim | ↑ | — |
| | (1 min before vs after onset) | | | |
| | LFP-stimulation pulse train phase locking | Baseline vs. stim | ↑ | ↑ |
| Behavior | % time in running | Baseline vs. stim | ↑ | ↑ |
| | Motion onset frequency | Baseline vs. stim | ↓ | — |
| | Speed spectral delta power | Baseline vs. stim | ↑ | — |
| LFP | LFP-speed phase locking | True vs. shuffled- baseline | ↑ | ↑ |
| | | and stim | | |
| | | Baseline vs. stim | ↑ | — |
| | Cross frequency coupling between speed- | True vs. shuffled- baseline | ↑ | ↑ |
| | delta and LFP-beta | and stim | | |
| | | Baseline vs. stim | ↑ | — |
| | Cross frequency coupling between speed- | True vs. shuffled | ↑ | ↑ |
| | delta and LFP-gamma | Baseline vs. stim | — | — |
| Calcium responses | Event rate | Resting - stim vs. baseline | — | — |
| | (Mixed effect models - Movement- | | | |
| | responsive neurons) | Running - stim vs. baseline | ↓ | ↓ |
| | Cross correlation - lag | Baseline vs. stim | — | — |
| | Cross correlation - correlation coefficient | Baseline vs. stim | — | — |
| Ca-movement | Ca²⁺ vs. delta-component of speed PLV | True vs. shuffled- baseline | ↑ | ↑ |
| | | and stim | | |
| | | Baseline vs. stim | — | — |
| Ca-LFP | Ca²⁺ vs. delta component of LFP PLV | True vs. shuffled- baseline | ↑ | ↑ |
| | | and stim | | |
| | | Baseline vs. stim | — | — |
| Pair-wise | Effect of Locomotion | % Correlated pairs | ↑ | ↑ |
| Correlation | | Correlation coefficient | ↓ | ↓ |
| | | across correlated pairs | | |
| | | Corr. Coeff. across all pairs | ↓ | ↓ |
| | | of movement-responsive | | |
| | | neurons | | |
| | | Event onset frequency | ↑ | ↑ |
| | Effect of stimulation during resting | % Correlated pairs | ↓ | ↓ |
| | | Correlation coefficient | ↑ | ↑ |
| | | across correlated pairs | | |
| | | Event onset frequency | ↑ | ↑ |
| | Effect of stimulation during running | % Correlated pairs | ↓ | ↓ |
| | | Correlation coefficient | ↑ | ↑ |
| | | across correlated pairs | | |
| | | Event onset frequency | — | — |

### Discussion of Experimental Examples 4-11

To understand the impact of beta-rhythmic sensory stimulation on stepping movement and basal ganglia circuits, we delivered audiovisual stimulation at either 10 Hz or 145 Hz to awake head-fixed mice voluntarily locomoting, while recording locomotion, striatal LFPs and cellular calcium dynamics. Consistent with the general ability for low frequency stimulation to entrain brain circuits, we found that sensory stimulation at 10 Hz, but not 145 Hz, effectively paced striatal population dynamics. While sensory stimulation at both frequencies promoted locomotion and desynchronized striatal network, only 10 Hz stimulation suppressed movement onset transitions, enhanced stepping rhythmicity at delta frequencies, and strengthened the coupling of LFP delta and beta oscillation power to the phase of delta-rhythmic stepping. Thus, beta-frequency sensory stimulation enhances gait rhythmicity by strengthening the interactions between striatal neural dynamics and stepping movement, distinct from the generally anti-kinetic effects observed with strong and artificial beta-frequency electrical stimulation, tACS or DBS, of cortical-basal ganglia circuits, highlighting the potential of beta-rhythmic sensory stimulation for improving gait.

We found that 10 Hz stimulation resulted in a significant increase in 10 Hz LFP spectral power, which lagged the stimulation onset for several seconds and remained elevated for a few seconds after offset. The delayed LFP entrainment effect suggests a network mechanism, and in particular the seconds long delay highlights the potential involvement of broad sensorimotor regions. Stepping movement exhibits characteristic delta frequencies in both mice and humans. Under our experimental conditions, mice movement rhythmicity peaked around 3-4 Hz (the delta frequencies analyzed here), which was enhanced by 10 Hz sensory stimulation, but not 145 Hz stimulation. While beta oscillations are conventionally thought to maintain the status quo and once exaggerated, as in Parkinson's disease, leads to akinesia³⁴, our results revealed that 10 Hz sensory stimulation heightens movement bouts, similar to that observed during 145 Hz stimulation. These findings are supportive of the idea that 10 Hz sensory stimulation can engage neuronal activities that occur during natural physiological processes, in contrast to the strong and artificial electrical or magnetic stimulation of the motor circuits that could evoke exaggerated oscillations that do not occur during physiological conditions. Thus, the heightened locomotion during sensory stimulation at both 10 Hz and 145 Hz is likely mediated by strengthened interactions between sensorimotor circuits. Nonetheless, the decrease in onset transitions observed during 10 Hz sensory stimulation is consistent with the general notion that beta oscillations could help maintain ongoing movement.

We found delta-rhythmic stepping movement is synchronized with striatal LFP delta oscillations, which temporally organizes LFP beta and gamma power regardless of sensory stimulation. This observation is consistent with our previous findings and further confirms that dorsal striatum plays an important role in orchestrating stepping movement. Even though audiovisual stimulation at both 10 Hz and 145 Hz promoted movement, only 10 Hz stimulation strengthened the coupling of striatal LFP delta rhythms and stepping. This enhanced coupling likely arises from the entrainment of thalamic and cortical sensory inputs to the striatum, as LFPs are dominated by synaptic inputs. However, proprioceptive inputs due to delta rhythmic movement could also contribute to the strengthened coupling between LFP delta rhythms and stepping.

It is intriguing that the faster beta oscillations augmented by sensory stimulation could regulate the slower delta oscillations to promote stepping automaticity. Neuronal delta rhythms and beta rhythms are temporally linked via cross-frequency coupling across motor circuits, but generally with the slower delta rhythms setting up the temporal window to modulate the power of the faster beta rhythms. Our recent study showed that many striatal neurons exhibit delta rhythmic membrane voltage fluctuations, with spike occurring at beta frequency time scales around the peak depolarization of the cellular membrane voltage delta oscillations. Thus, it is highly probable that the cellular mechanisms supporting the generation of delta and beta rhythms are linked, and thus selective enhancement of cellular beta rhythms could impact the slower delta rhythms. Future voltage imaging studies that directly measure the cellular effects of sensory stimulation could reveal insights on how higher beta frequencies could impact the lower delta frequencies and their coupling with stepping.

We used cellular GCaMP7f calcium imaging to probe the effect of sensory stimulation on individual striatal neurons and population striatal networks. To estimate neuronal spiking probability from cytosolic calcium that is directly measured GCaMP7f, we identified calcium events as those with sharp rising phases and performed our analysis with either event onset or the rising phase of the identified calcium events. We found that calcium event onsets are phase-locked to the delta component of both stepping movement and LFP, despite the slow kinetics of calcium imaging, consistent with our previous fast near kilohertz voltage imaging study. The temporal relationship of calcium events with movement and LFP delta rhythms were largely unaffected by sensory stimulation, in agreement with the fact that calcium events reflect cellular spiking output probabilities, highlighting that sensory stimulation boosts locomotion without interfering with striatal locomotion encoding ability.

Interestingly, we found that ˜70% of neurons exhibited increase in cellular calcium during movement, similar to prior electrophysiological studies, but much greater than previous calcium imaging studies using GCaMP6f, most likely due to the improved sensitivity of GCaMP7f at capturing movement related cellular responses than GCaMP6f. Further, consistent with previous electrophysiology studies, striatal cellular calcium rises preceded locomotion by approximately a hundred milliseconds, supporting the role of striatum in regulating movement. Upon sensory stimulation, individual striatal neurons exhibited diverse responses, with some increasing their activity rate and others decreasing. However, we found an equal proportion of activated and inhibited fractions across the population regardless of locomotion states, suggesting potential network compensation to maintain balanced excitability across behavioral states.

We observed a sharp and transient increase of calcium activity at stimulation onset, and a transient rebound at stimulation offset. When focusing on movement-responsive neurons that are relevant for the locomotion task condition, we found that sensory stimulation has an inhibitory effect when mice were running, but not resting. It is possible that it is easier to detect inhibition when these movement-responsive neurons have higher activity rate during running in contrast to resting. Nonetheless, our results are consistent with previous studies showing sensory stimulation generates smaller responses in medium spiny projecting neurons (SPNs) during up-state than down-state in anesthetized mice, and the observed sensory suppression of spontaneous whisking-evoked striatal neuron activity in awake mice. Previous studies also reported differential sensory responses between D1-SPNs and D2-SPNs. It is possible that some of the heterogeneity observed here arises from the systemic difference between striatal neuron subtypes. In addition to SPNs, striatal cholinergic interneurons, known to be critical in regulating striatal dynamics through its broad axonal projections, may be robustly recruited by sensory stimulation. Similarly, parvalbumin-positive fast spiking interneurons and other GABAergic interneurons have also been shown to be critical in sensorimotor behaviors. Future cell type specific analysis will offer insights on how sensory stimulation engages different striatal neuron subtypes and their downstream targets.

At the network level, we found that sensory stimulation and locomotion both desynchronized movement-responsive neuron populations, though through distinct effects as revealed with the pairwise correlation analysis. During locomotion, we observed an increase in the proportion of correlated neuron pairs, similar to previous findings of increased functional connectivity within neuron clusters during movement. However, the connectivity strength between these correlated neuron pairs was lower during running compared to resting, pointing to a desynchronization effect. Desynchronization increases network information coding, which would be consistent with elevated processing of the environment as animals move, even though under our experimental condition mice were head fixed. During stepping, although striatal neurons' calcium event rates increased, we detected a reduction in pairwise correlation coefficients, that may stem from greater asynchrony between calcium event timing across neurons, which could arise from many mechanisms, including non-overlapping inputs to striatal neurons, lateral inhibition between striatal neurons, or temporally jittered spiking outputs to membrane depolarizations. During sensory stimulation, the heightened sensory inputs to striatum could engage similar cellular and network mechanisms to enhance network information coding by reducing neuronal synchrony. While sensory stimulation reduced the fraction of correlated cell pairs in the movement-responsive striatal network, the correlation strength within these neurons increased, suggesting that sensory inputs have the potential to synchronize specific neuron populations receiving joint multimodal inputs. Overall, the sensory stimulation evoked desynchronization supports a potential therapeutic benefit in boosting movement by making the striatal network more dynamic and flexible.

There is natural decline in gait automaticity with aging, and in particular Parkinson's disease is characterized by various gait impairments, including heightened temporal variability and asymmetry, diminished rhythmicity, and shortened step length. The malfunction of basal ganglia circuits in Parkinson's disease is thought to disrupt habitual or automatic process, resulting in heightened cognitive effort during stepping and loss of gait automaticity. To compensate for the diminished gait automaticity, Parkinsonian patients often rely on sensory cues during walking. It has been suggested that sensory cues help improve gait rhythmicity by regularizing inputs to provide a temporal framework during stepping movement. Further, it has been suggested that abnormal neuronal synchrony in Parkinson's disease may result from a loss of active decorrelation within the basal ganglia, and our previous study linked abnormal increase in striatal synchrony to motor deficits in low-dopamine conditions. The observation that sensory stimulation desynchronizes striatal network also supports a desynchronization mechanism underlying heightened locomotion during sensory stimulation. Our study highlights the potential of using non-invasive sensory stimulation to influence various aspects of movement, including stepping rhythmicity, the propensity to move, and movement onset transitions.

### Example 12—Optical Voltage Imaging Reveals that Most Cholinergic Interneurons (ChIs), and a Subset of Non-Specific Striatal Neurons (SYNs), Exhibit Spike Bursting at Delta Frequencies (1-4 Hz) During Locomotion

To examine whether single cell voltage rhythmicity is directly coupled to behavioral rhythms, experiments focusing on delta-frequencies (1-4 Hz), which are known to occur at both neural network and behavioral levels, were studied. Membrane voltage imaging of individual striatal neurons was performed simultaneously with network-level local field potential recordings in mice during voluntary movement. Sustained delta oscillations in the membrane potentials of many striatal neurons were observed, particularly cholinergic interneurons, which organize spikes and network oscillations at beta frequencies (20-40 Hz) associated with locomotion. Furthermore, the delta-frequency patterned cellular dynamics are directly coupled to animals' stepping cycles. Thus, delta-rhythmic cellular dynamics in cholinergic interneurons known for their autonomous pace-making capabilities may play a direct role in regulating network rhythmicity and movement patterning.

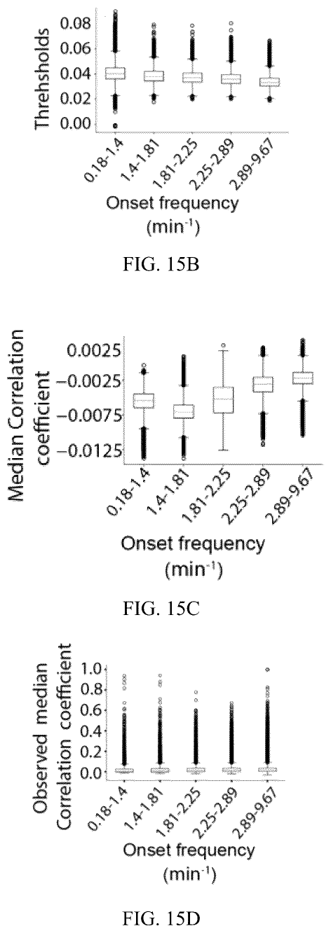

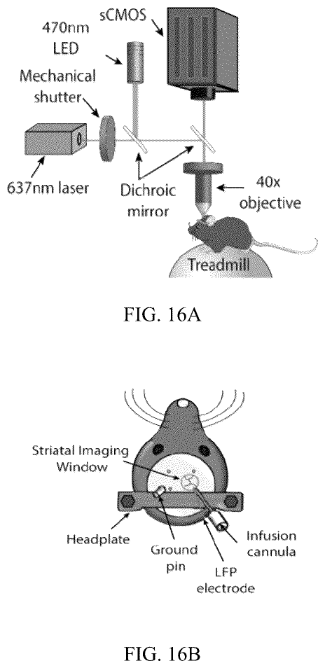

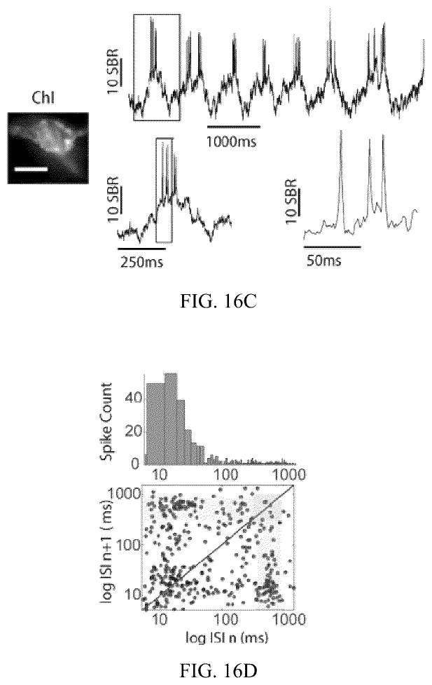

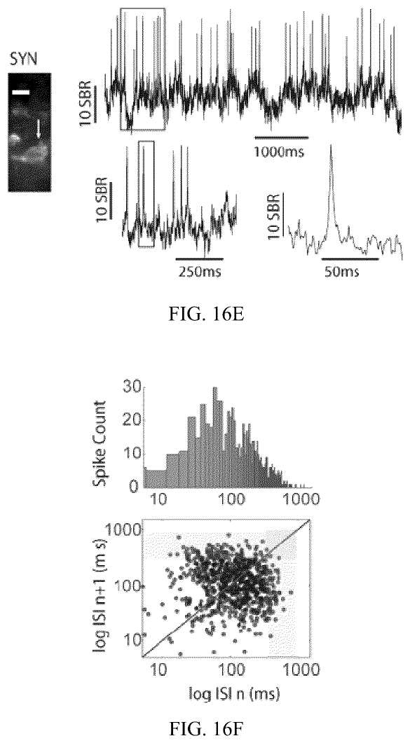

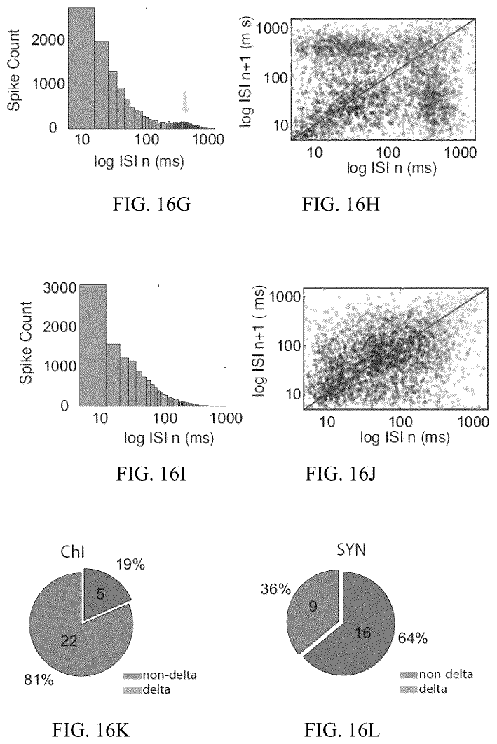

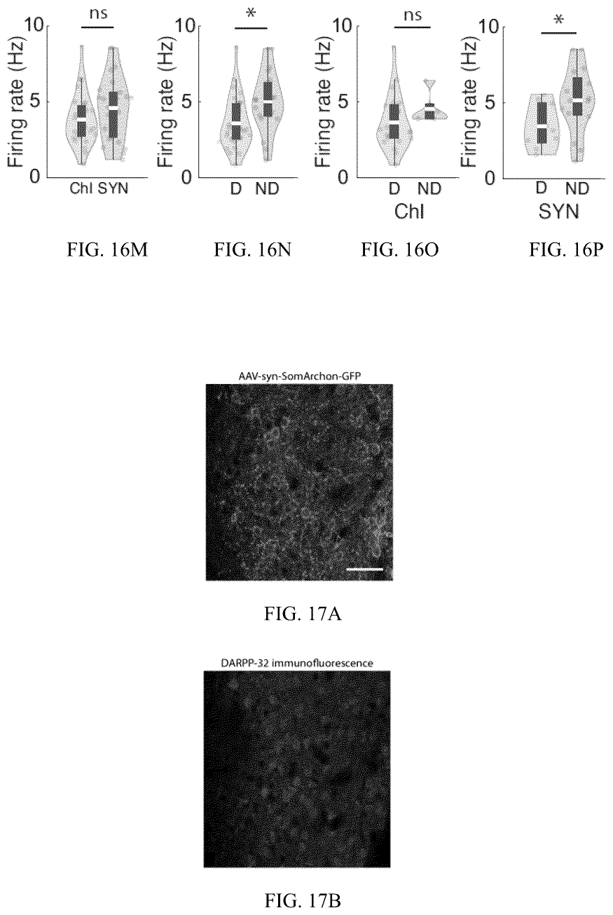

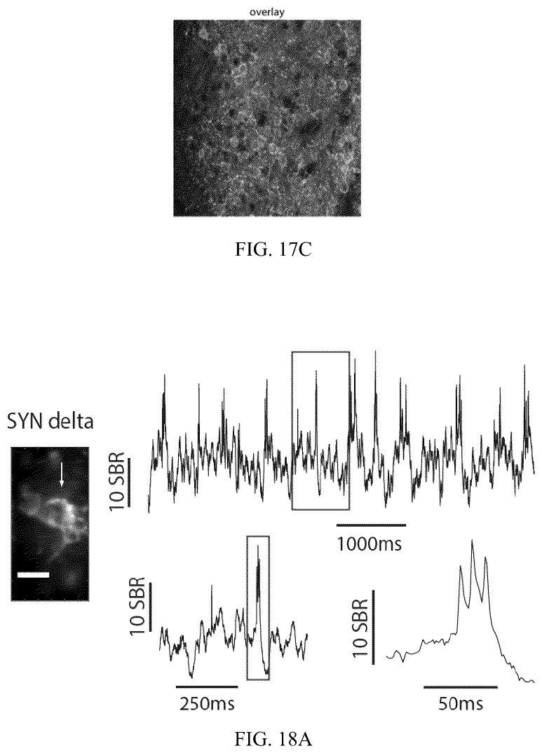

To measure membrane voltage from individual striatal neurons along with local field potentials (LFPs) during locomotion, we surgically implanted custom imaging windows coupled with an infusion cannula and an LFP electrode over the dorsal striatum (FIGS. 16A-16B). AAV viral vectors were then infused through the infusion cannula to transduce individual striatal neurons with the genetically encoded, soma-targeted voltage indicator SomArchon (FIG. 16C, 16E, left). SomArchon voltage imaging of cholinergic interneurons (ChIs) was performed by either infusing Cre-dependent AAV-FLEX-SomArchon-GFP into ChAT-Cre mice or using tdTomato (tdT) fluorescence to identify ChIs in ChAT-tdT mice infused with AAV-syn-SomArchon-GFP (n=6 mice). To compare ChIs to the general striatal neuron population, we infused AAV-syn-SomArchon-GFP into the striatum to transduce neurons expressing synapsin (SYNs), a non-specific neuronal marker (n=7 mice). Histological quantification confirmed that 84.5±7.2% (mean±standard deviation, n=34 fields of view in 17 brain slices from 4 mice, containing a total of 836 neurons) of transduced SYNs were SPNs, expressing the SPN-specific protein DARPP-32 (FIGS. 17A-17C) similar to previous observations.

FIG. 16A and FIG. 16B show illustrations of the (FIG. 16A) experimental setup and (FIG. 16B) surgically-placed imaging window. FIG. 16C shows an example ChI's SomArchon fluorescence trace recorded at 833 Hz (left bottom, mean SomArchon fluorescence of the recorded neuron). Right, example SomArchon fluorescence trace, with a zoom-in view (below) of the period indicated by the green box, and a further zoom-in (right). FIG. 16D shows, at the top, the inter-spike interval (ISI) distribution of the example ChI shown in FIG. 16C, at the bottom, the ISI return map. The delta frequency range is illustrated with orange shading. FIG. 16E is the same as FIG. 16C, but for an example SYN. FIG. 16F is the same as FIG. 16D, but for the example SYN shown in FIG. 16E. Scale bars are 15 μm. FIG. 16G shows the population ISI distribution of delta-rhythmic neurons (n=31). Arrow indicates the delta time-scale ISI distribution peak. FIG. 16H shows the ISI return map of all delta-rhythmic neurons (n=31). FIG. 16I and FIG. 16J show the population ISI distribution (FIG. 16I) and ISI return map (FIG. 16J) of non-delta neurons (n=21). FIG. 16K and FIG. 16L show pie chart illustrations of the fraction of (FIG. 16K) ChI neurons and (FIG. 16L) SYN neurons identified as delta-rhythmic or non-delta. FIG. 16M shows population firing rate for ChIs and SYNs. There was no difference between ChIs and SYNs (independent t-test, p=0.16, n=52, with 27 ChIs and 25 SYNs, df=50). FIG. 16N shows population firing rate for delta-rhythmic (D) and non-delta (ND) neurons (independent t-test, p=0.007, n=52, with 31 delta-rhythmic neurons and 21 non-delta neurons, df=50). FIG. 16O shows mean firing rate quantification for delta-rhythmic and non-delta ChIs (independent t-test, p=0.26, n=27, df=25). FIG. 16P is the same as FIG. 16O, but for SYNs (independent t-test, p=0.04, n=25, df=23). Quantifications in FIGS. 16M-16P are visualized as violin plots with the outer shape representing the data kernel density and a box-and-whisker plot (box: interquartile range, whiskers: 1.5× interquartile range, white line: mean). All statistical tests are two-sided. Source data are provided as a Source Data file.

FIG. 17A shows an example dorsal striatal brain slice, showing GFP fluorescence (λ=488 nm) in neurons expressing SomArchon-GFP fusion proteins transduced with AAV-syn-SomArchon in a C57BL6 mouse. FIG. 17B shows immunofluorescence of DARPP-32 (AlexaFluor568, λ=568 nm), an SPN-specific neuronal marker, in the same brain slice as FIG. 17A. FIG. 17C shows an overlay of FIG. 17A and FIG. 17B. Scale bar is 50 μm.

Striatal LFP recordings and ChI and SYN single cell membrane voltage traces were obtained as head-fixed mice ran voluntarily on a treadmill made of a ball freely rotating along the center axis FIG. 16A). Due to the need for high imaging speed to capture the millisecond time-scale membrane voltage fluctuations in individual neurons, voltage imaging was performed with a custom widefield microscope at a frame rate of 833 Hz (FIG. 16A). During each recording, we first identified SomArchon-expressing cells based on the fluorescence of soma-targeted GFP (fused to SomArchon) under a 10× objective. We then collected near-infrared SomArchon fluorescence from the identified neurons under a 40× objective lens to maximize photon collection efficiency. We restricted the illumination of the near-infrared 637 nm laser to a circular area of ˜70 μm in diameter to minimize background SomArchon fluorescence excitation under the widefield microscopy configuration, resulting in a field of view (FOV) size of about 50 μm×70 μm.

During offline data analysis, we first performed motion correction of the recorded video to correct fine image motion, inherent to imaging studies in awake behaving animals (details of experimental methods discussed below). Since SomArchon expression is restricted to the soma and proximal dendrites, with little detectable expression in dendrites beyond 30 μm from the soma, we manually segmented the cell body based on SomArchon fluorescence and extracted SomArchon fluorescence traces. SomArchon fluorescence traces were visually inspected to ensure minimal image motion and reasonable signal-to-noise level based on spike appearance. Trials with low spike signal-to-noise were removed and time periods with image motion exceeding 0.065 μm/s were excluded from further analysis. We then identified spikes from SomArchon membrane voltage traces and subsequently calculated the subthreshold membrane voltage trace (Vm) at the soma by removing the identified spikes.

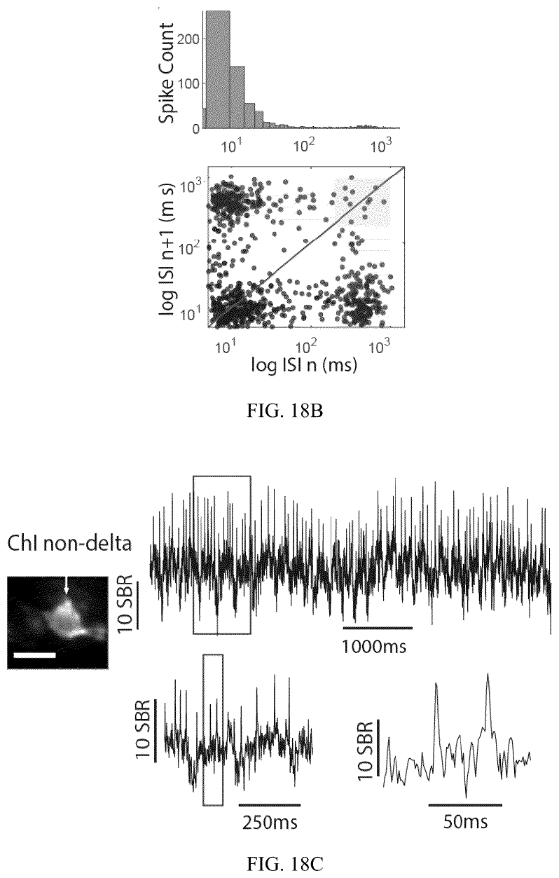

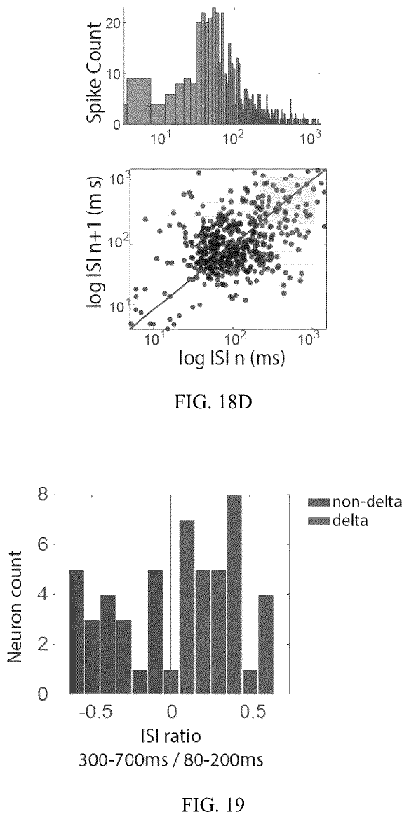

ChIs are known to be tonically active, and we indeed detected persistent spiking across all recorded ChIs, with many exhibiting delta-rhythmic (1-4 Hz) spike bursting accompanied by large Vm depolarizations (FIG. 16C). To quantify spike rhythmicity, we calculated the inter-spike interval (ISI) distribution across all spikes for each recorded neuron (representative ChI and SYN in FIGS. 16D, 16F, respectively). In most ChIs and a small subset of SYNs, we detected two ISI peaks: one centered around 10-40 ms corresponding to high-frequency spike bursting at 25-100 Hz, and the other at approximately 300-700 ms (1.4-3.3 Hz) largely within the delta frequency range (FIG. 16D; FIGS. 18A-18B). The remaining majority of SYNs and the few remaining ChIs, however, exhibited more regularly spaced spiking patterns (FIG. 16E, FIG. 16F; FIG. 18C, FIG. 18D).

FIG. 18A illustrates an example SomArchon fluorescence recording from a delta-rhythmic SYN (sampling rate at 826 Hz). In FIG. 18A, at left, SomArchon fluorescence image, with the example neuron indicated by an arrow. At right, 6 seconds long SomArchon fluorescence trace is shown for the example SYN, with sequential zoom in of the periods indicated by the dashed green boxes. In FIG. 18B, at the top, the inter-spike interval (ISI) distribution of the example SYN shown in FIG. 18A. At the bottom, the return map, plotted as ISI (n) vs ISI (n+1), for the example SYN shown in FIG. 18A. Orange shading indicates the delta rhythmic band. The X- and Y-axis are in logarithmic scale. The bin size in the top ISI distribution is fixed at 5 ms. FIG. 18C is the same as FIG. 18A, but for an example non-rhythmic ChI. FIG. 18D is the same as FIG. 18B, but for the example neuron shown in FIG. 18C. Scale bars are 15 μm. Source data are provided as a Source Data file.

Given the prominence of delta rhythmic spike bursting observed in many recorded neurons, we categorized each ChI and SYN as either delta-rhythmic (‘delta’) or non-delta-rhythmic (‘non-delta’) based on the ratio of the fraction of ISIs at 300-700 ms to the fraction of ISIs at 80-200 ms (FIG. 19), a measure that captures the delta rhythmicity. The classified neurons' ISI return maps, which plots the inter-spike interval relative to the subsequent spike (ISI n vs. ISI n+1), were also manually inspected to confirm delta rhythmicity. Across the delta neuron population, the ISI distribution map had a prominent peak around delta frequency (2-3 Hz) (FIG. 16G, FIG. 16H), while non-delta neurons did not exhibit a delta frequency ISI peak (FIG. 16I, FIG. 16J). We found that 81% of ChIs exhibited delta-rhythmic behavior, compared to only 36% of SYNs (FIG. 16K, FIG. 16L). While the overall firing rate of ChI and SYN populations were similar (FIG. 16M), delta-rhythmic neurons as a population had a significantly lower firing rate than non-delta neurons (FIG. 16N). While this effect was less pronounced in ChIs (FIG. 16O), delta-rhythmic SYNs, in particular, had significantly lower firing rates than non-delta SYNs (FIG. 16P). For both ChIs and SYNs, delta-rhythmic neurons had inter-burst firing rates at delta frequency as expected (ChIs: 2.28±0.034 Hz, SYNs: 2.27±0.039 Hz).

FIG. 19 shows the delta-rhythmicity in the inter-spike intervals (ISI) was quantified for each neuron as the ratio (A-B)/(A+B) between the average ISI probability over the 300-700 ms range (A) and the averaged ISI probability quantified over the 80-200 ms range (B). An ISI ratio threshold of 0 is used to determine whether a neuron was classified as non-delta (blue, ND, n=21) or delta-rhythmic (red, D, n=31). Source data are provided as a Source Data file.

### Example 13—Delta Rhythmic Striatal Neurons Exhibit Prominent Subthreshold Membrane Voltage (Vm) Delta Oscillations that Organize Spike Timing

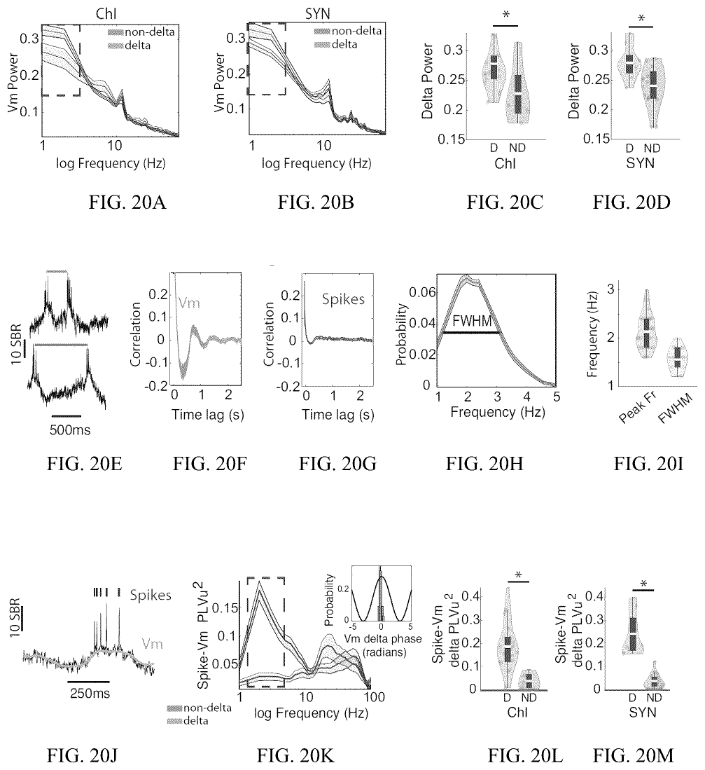

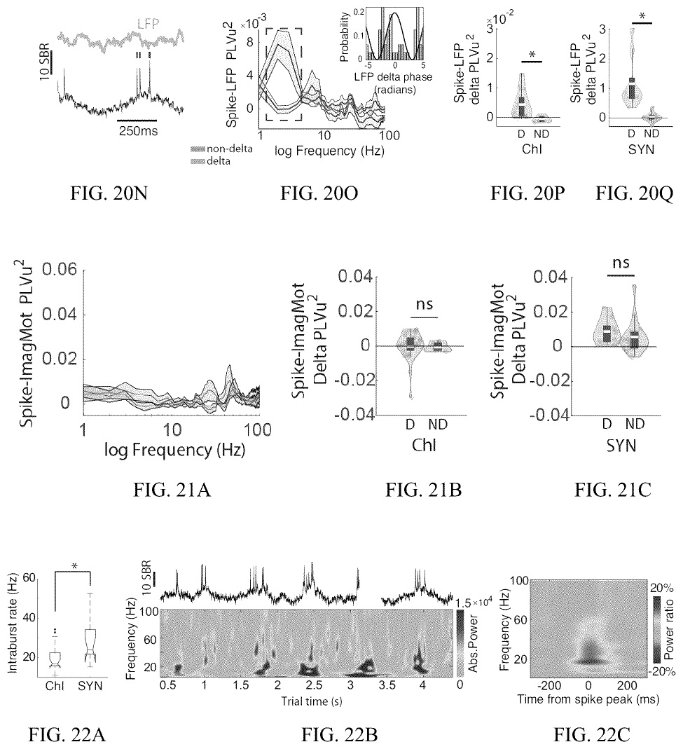

Spike timing critically depends on membrane voltage dynamics, and we noticed spike bursting in delta-rhythmic neurons was often associated with prominent somatic Vm depolarizations at delta frequencies (FIG. 16C). Indeed, the time-averaged Vm wavelet power of delta-rhythmic neurons had a significantly higher delta power than non-delta neurons (FIG. 2a,b), for both ChI and SYN populations (FIG. 20C, FIG. 20D). Further, we found substantial variability of the peak delta frequency over time (FIG. 2e) resulting in weak delta timescale autocorrelograms (FIG. 20F, FIG. 20G). Since delta frequencies are low, slight variation within the delta frequency range could result in large differences in cycle length ranging from around 0.25s to 1s. We thus further examined the distribution of instantaneous delta frequency across delta cycles (peak frequency at 2.13±0.36 Hz) and quantified the delta frequency variability using the full-width-at-half-maximum of the frequency distribution (FWHM, 1.55 Hz±0.24, FIG. 20H, FIG. 20I). The large variation in instantaneous delta frequencies could explain the lack of obvious spike rhythmicity of ChIs in previous extracellular recording studies that characterized putative ChIs as tonically active neurons (TANs) with wide extracellular spike waveforms.

To further investigate how the delta oscillations we observed relate to spike timing and higher frequency oscillations, we calculated the squared phase locking value (PLVu²; unbiased by the number of spikes, see Methods) of spikes relative to Vm (FIG. 20J, FIG. 20K) and LFP oscillations (FIG. 20N, FIG. 20O). For both ChIs and SYNs, spike-Vm PLVu² at delta frequency peaks (2-3 Hz) was significantly higher for delta-rhythmic neurons than non-delta neurons (FIG. 20L, FIG. 20M). Further, while LFP oscillations in the striatum are weak due to the lack of generally organized dendritic configuration among striatal neurons, we found spike-LFP PLVu² (FIG. 20N, FIG. 20O) to be the strongest in the same 2-3 Hz delta frequency range as for Vm for both ChIs and SYNs. As with Vm, spikes in delta neurons were significantly more phased locked (higher PLVu²) than non-delta neurons to these LFP delta oscillations (FIG. 20P, FIG. 20Q). As breathing and other biological motion may also occur around delta frequencies, we examined whether the observed spike phase locking to Vm and LFP delta may be related to image motion that is inherent in fluorescence imaging studies. We computed PLVu² of spikes to image motion, calculated as the imaging frame shift during motion correction (details in Methods). We found that spike-image motion PLVu² was generally weak, with no difference between delta-neurons and non-delta neurons, and no noticeable peaks around the delta frequencies (FIGS. 21A-21C). Thus, spiking coordination with Vm or LFP delta rhythms cannot be explained by potential image motion. Taken together, these results demonstrate that many striatal neurons, in particular ChIs, exhibit prominent Vm delta oscillations, which organize spike timing that is also coordinated with network level LFP delta oscillations.

FIGS. 20A-20Q illustrate subthreshold membrane voltage (Vm) delta rhythm structures spike timing and Vm. FIGS. 20A-20B show population Vm spectral power of (FIG. 20A) delta-rhythmic ChIs (red, n=22) and non-delta ChIs (blue, n=5) and of (FIG. 20B) delta-rhythmic SYNs (red, n=9) and non-delta SYNs (blue, n=16). Boxes highlight the delta frequencies. FIG. 20C shows delta power (1-4 Hz) for delta-rhythmic (D) and non-delta (ND) ChIs (independent t-test, p=0.047, n=27, df=25). FIG. 20D is the same as FIG. 20C, but for SYNs (independent t-test, p=0.006, n=25, df=23). FIG. 20E shows an illustration of delta-cycle length variability in an example ChI. FIGS. 20F-20G shows the population (FIG. 20F) Vm and (FIG. 20G) spike autocorrelogram for delta-rhythmic ChIs (n=22). FIG. 20H shows population-averaged instantaneous Vm delta-frequency distribution (1-6 Hz was used to better capture the variations in instantaneous delta-frequencies). FWHM: full-width-at-half-maximum. FIG. 20I shows quantification of peak delta frequency and FWHM of delta-frequency distribution (n=31). FIG. 20J shows an example striatal neuron's Vm filtered at 1-4 Hz (yellow) and spikes (red ticks). FIG. 20K shows a squared phase-locking value (PLVu²) of spikes to Vm across frequencies for delta-rhythmic (red) and non-delta neurons (blue). Inset: the polar histogram of an example delta-rhythmic neuron's preferred spike phase to Vm delta. FIG. 20L shows spike-Vm PLVu² around peak delta frequency (2-3 Hz) in delta-rhythmic (D, red) versus non-delta ChIs (ND, blue) (independent t-test, p=0.005, n=27, df=25). FIG. 20M is the same as FIG. 20L, but for SYNs (independent t-test, p=3.08e⁻⁸, n=25, df=23). FIG. 20N shows example voltage trace of a striatal neuron with spikes (red ticks) and corresponding LFP (yellow). FIG. 20O shows PLVu² of spikes to LFP across frequencies for delta-rhythmic (red) and non-delta neurons (blue). Inset: the polar histogram of delta-rhythmic neurons' preferred spike phase (mean angle) to LFP delta oscillations. FIG. 20P shows spike-LFP PLVu² in the delta frequency range between delta-rhythmic ChIs (red) and non-delta ChIs (independent t-test, p=0.032, n=27, df=25). FIG. 20Q is the same as FIG. 20P, but for SYNs (independent t-test, p=4.84e⁻⁵, n=25, df=23). All shaded regions around line plots represent standard error of mean. Quantifications are visualized as violin plots with the outer shape representing the data kernel density and a box-and-whisker plot (box: interquartile range, whiskers: 1.5× interquartile range, white line: mean). All statistical tests are two-sided. Source data are provided as a Source Data file.

FIGS. 21A-21C show that the relationship between spikes and image motion is weak and not different between neuron groups. FIG. 21A shows an estimation of squared phase-locking value (PLVu2) of spikes to image motion displacement for delta-rhythmic (red) or non-delta neurons (blue). Image motion displacement calculated as frame shift during motion correction (√XX2+YY2image pixel displacement), corresponding to the spatial shift of neurons within an FOV, inherent for awake, behaving conditions with intrinsic biological motion e.g., breathing, heartbeat, movement. Instrument noise (vibration of cooling mechanical fan for sCMOS camera) was also observed around 80 Hz. Solid lines represent the population means of delta-rhythmic (red, n=31) and non-delta (blue, n=21) neurons. Phase-locking of image motion with delta-rhythmic vs. non-delta neuron spiking at delta frequency was not significantly different. FIG. 21B shows quantification of the PLVu2 for spikes to image motion displacement at delta frequency (1-4 Hz) between delta-rhythmic ChIs (D, red) and non-delta ChIs (ND, blue, independent t-test, ChI, D vs ND: p=0.94, n=27, df=25). FIG. 21C is the same as FIG. 21C, but for SYNs (independent t-test, SYN, D vs ND: p=0.43, n=25, df=23). Shaded regions around lines represent standard error of mean. Violin plots represent the data kernel density with a box-and-whisker plot (box: interquartile range, whiskers: 1.5× interquartile range, white line: mean). All statistical tests are two-sided. Source data are provided as a Source Data file.

### Example 14—the Phase of Vm Delta Oscillations is Coupled to Beta Power Modulation in Both Vm and LFP

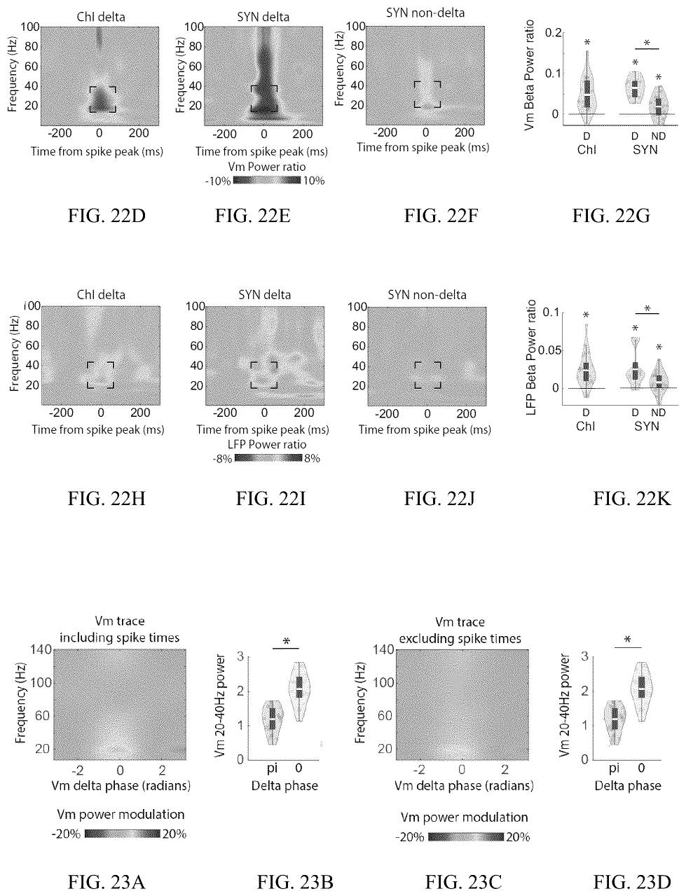

FIGS. 22A-22K illustrate subthreshold Vm delta rhythm structures Vm and LFP beta power. FIG. 22A shows mean intra-burst firing frequency for delta-rhythmic ChIs and SYNs (independent t-test, p=0.007, n=31, df=29). FIG. 22B shows example voltage trace of a delta-rhythmic striatal neuron with corresponding Vm wavelet power spectrum shown below. FIG. 22C shows mean normalized Vm spectral power aligned to the peak of all spikes in the example neuron shown in FIG. 22B. FIGS. 22D-22F shows Vm power around spike peak relative to pre-spike period (−200 to −100 ms) for FIG. 22D Delta-rhythmic ChIs (n=21), FIG. 22E Delta-rhythmic SYNs (n=9), and FIG. 22F Non-delta rhythmic SYNs (n=16). FIG. 22G shows quantification of Vm power around spike peak relative to pre-spike period at beta-frequencies (20-40 Hz) for delta-rhythmic (D, red) and non-delta neurons (ND, blue), ChI (left) and SYN (right). Vm beta power at spike peak was significantly higher than pre-spike period in all groups (all one-sample t-test, ChI, D: p=1.7×10⁻⁵, df=21; SYN, D: p=5.3×10⁻⁵, df=8; SYN ND: p=0.015, df=15). Delta-rhythmic SYNs showed greater modulations than non-delta SYNs (independent t-test SYN D vs SYN ND: p=3.64×10⁻⁴, n=25, df=23). FIGS. 22H-22J show spike-aligned LFP power (relative to pre-spike period) for FIG. 22H Delta-rhythmic ChIs (n=21), FIG. 22I Delta-rhythmic SYNs (n=9), and FIG. 22J Non-delta SYNs (n=16). FIG. 22K shows quantification of LFP power around spike peak relative to pre-spike period at beta-frequencies for delta-rhythmic (red) and non-delta neurons (blue), ChI (left) and SYN (right). LFP beta power at spike peak was significantly higher than pre-spike period in all groups (all one-sample t-test, ChI D: p=8.9×10⁻⁵, n=22, df=21; SYN, D: p=0.0147, n=9, df=8; SYN, ND: p=0.04, n=16, df=15), and delta-rhythmic SYNs showed greater modulations than non-delta SYNs (independent t-test SYN D vs SYN ND: p=0.037, n=25, df=23). Quantifications are visualized as violin plots with the outer shape representing the data kernel density and a box-and-whisker plot (box: interquartile range, whiskers: 1.5× interquartile range, white line: mean). All statistical tests are two-sided. Source data are provided as a Source Data file.

In delta rhythmic ChIs and SYN neurons, we found that the intra-burst firing rate of delta-rhythmic neurons was centered around the beta frequency range (ChIs: 21±1.5 Hz, SYNs: 27.3±3.57 Hz; FIG. 22A), suggesting that delta-rhythmic firing patterns might be associated with beta rhythms. When we aligned Vm wavelet spectrum power to individual SomArchon voltage traces, we noticed that spikes were indeed accompanied by an increase in Vm beta-band (20-40 Hz) power (FIGS. 22B-22C). Vm beta-band power was significantly higher around spikes in delta-rhythmic neurons for both ChI and SYN populations (FIG. 22D, FIG. 22E, and FIG. 22G), though the increase is much less pronounced in non-delta SYNs FIG. 22F, FIG. 22G). Non-delta ChIs were not examined due to low neuron number. To understand whether the increase in Vm beta power was related to spikes occurring at beta frequencies within a burst, we separated the Vm delta cycles into periods that included spikes versus those without, and found that Vm beta power remained elevated at the peaks of delta oscillations even in the absence of spiking (FIG. 23A-23F). Thus, Vm delta-beta cross-frequency coupling is an intrinsic feature of Vm dynamics and is not due to delta-rhythmic spike bursting.

Similar analysis using LFP spectrum power showed that spikes in delta-rhythmic neurons were also coupled to an increase in LFP beta power for both ChI and SYN populations (FIG. 22H, FIG. 22I, FIG. 22K), though less prominent in non-delta rhythmic neurons (FIG. 22J, FIG. 22K). Together, these results demonstrate that cellular Vm delta-beta cross frequency oscillations organize beta frequency synchronization at the single neuron membrane voltage level. Further, spiking in striatal neurons that exhibit delta-rhythmicity are selectively coupled to prominent LFP delta and beta oscillations, suggesting that synchronization among delta-rhythmic neurons contributes to fluctuations in striatal LFP delta and beta power.

FIGS. 23A-23D illustrate that Vm delta-phase dependent beta power is independent of spiking activity. FIG. 23A shows normalized Vm spectrum power aligned to Vm delta-peak (relative to pre-peak period of −200 to −100 ms) in delta-rhythmic neurons (including both ChIs and SYNs, n=31) across all delta cycles and (FIG. 23C) only during delta cycles without any spikes. FIG. 23B shows quantification of Vm beta-band power (20-40 Hz) at Vm delta-peak (phase=0°) versus delta trough (phase=180°/pi). Vm beta power at delta peaks is significantly higher than that at delta troughs (paired t-test, n=31, df=30, with spikes: p=1.15e−15). FIG. 23C is the same as FIG. 23A, but only averaged across delta cycles without any spikes. FIG. 23D is the same as FIG. 23B, but for delta cycles without spikes. Vm beta power at delta peaks is significantly higher than that at delta troughs (paired t-test, n=31, df=30, without spikes: p=1.44e−15). Overall, Vm beta power was very similarly modulated by Vm delta phase regardless of spikes occurrence, suggesting that spiking is not required for Vm beta power modulation. Quantifications are visualized as violin plots with the outer shape representing the data kernel density and a box-and-whisker plot (box: interquartile range, whiskers: 1.5× interquartile range, white line: mean). All statistical tests are two-sided. All statistical tests are two-sided. Source data are provided as a Source Data file.

### Example 15—Delta-Rhythmic Neurons are Uniquely Activated During High-Speed Movement and at Movement Transitions

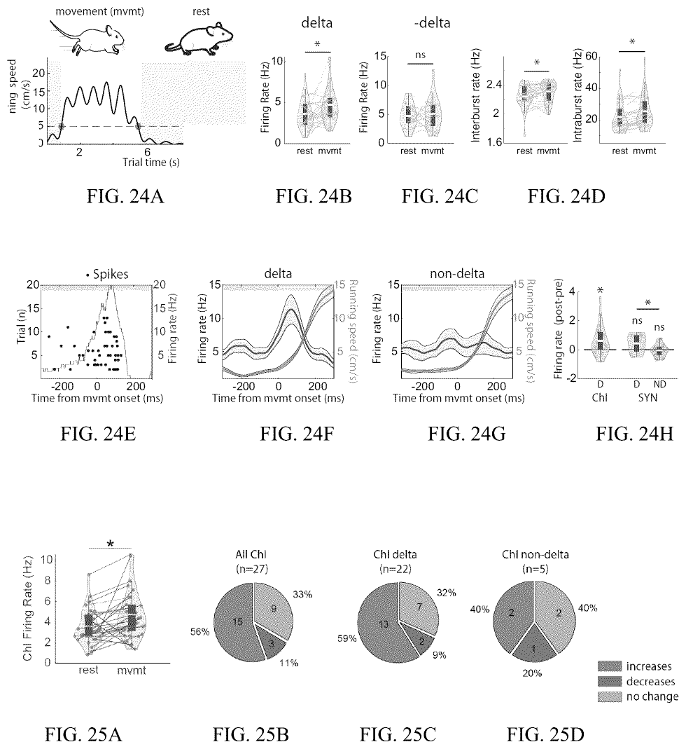

FIGS. 24A-24H show that delta-rhythmic neurons, but not non-delta neurons, exhibit locomotion-dependent spiking. FIG. 24A illustrates an example locomotion speed recording (black line) while an animal was voluntarily running on the treadmill. The dashed blue line indicates the threshold (5 cm/s) used to classify periods of rest (pink) and movement (green).

Blue dots mark the onset and offset of movement periods. The schematic resting mouse was adapted from https://doi.org/10.5281/zenodo. 3925949 and running mouse from https://doi.org/10.5281/zenodo.3925901. FIG. 24B shows firing rates in delta-rhythmic neurons during rest versus movement (paired t-test, p=0.0026, n=31, df=30). FIG. 24C is the same as FIG. 24B, but for non-delta neurons (paired t-test, p=0.556, n=21, df=20). In FIG. 24D, at left: Interburst firing rates of delta-rhythmic neurons during rest versus movement (paired t-test, p=0.005, n=31, df=30). Right: Intraburst firing rates of delta-rhythmic neurons during rest versus movement (paired t-test, p=1.7×10−5, n=31, df=30). FIG. 24E shows a spike raster plot aligned to movement onset for an example ChI. Blackpoints are spike times, and the red line indicates the average firing rate across all movement onsets. FIGS. 24F-24G show population firing rate aligned to movement onset for (FIG. 24F) delta-rhythmic neurons (n=31) and (FIG. 24G) non-delta neurons (n=21). Turquoise lines indicate running speed. FIG. 24H shows quantification of movement onset-related firing rate changes calculated as post-onset (0 to 120 ms) minus preonset (−120 to Oms). Only delta-rhythmic (D) ChIs increased their firing rates at onset (all one-sample t-test, ChI, D: p=0.019, n=21, df=20, one neuron excluded due to lack of movement data; SYN, D: p=0.099, n=9, df=8; SYN, non-delta (ND): p=0.53, n=16, df=15).

Delta-rhythmic SYNs had significantly stronger firing enhancement than non-delta SYNs (independent t-test SYNDvs SYNND: p=0.041, n=25, df=23). The shaded regions around line plots represent the standard error of the mean. Quantifications are visualized as violin plots with the outer shape representing the data kernel density and a box-and-whisker plot (box: interquartile range, whiskers: 1.5× interquartile range, white line: mean). All statistical tests are two-sided. Source data are provided as a Source Data file.

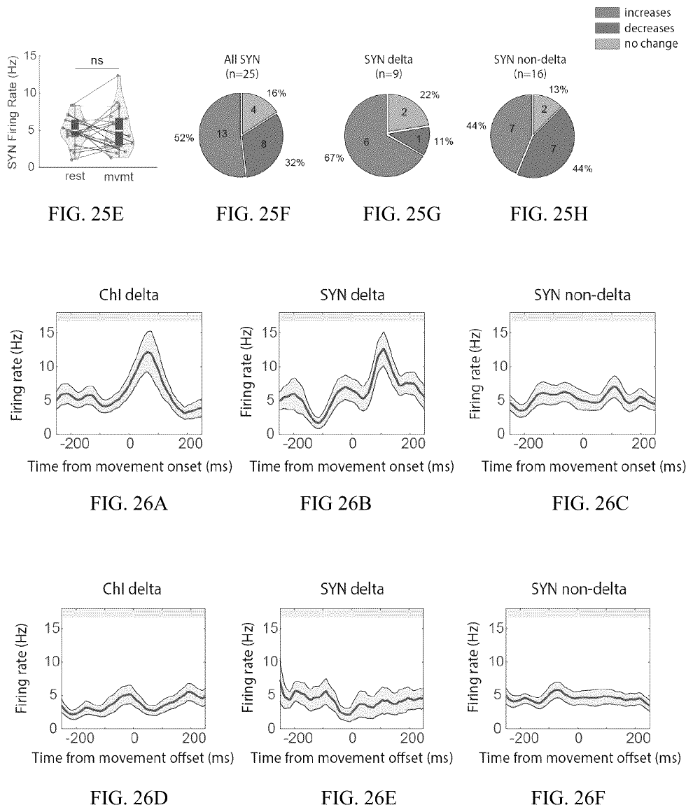

As striatal neurons and striatal LFP are modulated by various aspects of locomotion, we examined whether delta rhythmic vs. non-delta rhythmic neurons exhibited different locomotion-related responses. To assess locomotion-related responses, we classified stages of motor output into movement bouts (defined as treadmill speed≥5 cm/s), resting bouts (treadmill speed<5 cm/s), and movement onset and offset transitions between these movement bouts (FIG. 24A). We found that delta-rhythmic neurons significantly increased their firing rates as a population during movement compared to during rest, while non-delta neurons did not change their firing rates (FIG. 24B, FIG. 24C). Interestingly, delta-rhythmic neurons' interburst and intraburst spike frequency both increased during movement, as compared to resting periods (FIG. 24D). When we categorized these neurons based on cell type, we found that SYNs exhibited more heterogeneity in their movement responses than ChIs, and thus SYNs as a population were insensitive to movement speed while ChIs as a population increased their firing rate during movement (FIGS. 25A-25H).

FIGS. 25A-25H show that the ChI population, but not SYN population, increased firing rate during movement compared to rest. FIG. 25A shows mean firing rates of ChIs during rest vs. movement (mvmt). Red lines mark neurons with >10% firing rate increase during movement compared to rest. Blue lines mark neurons with <10% decreased firing rates, and gray lines mark neurons that are unchanged (<10% modulation in either direction). ChIs as a population significantly increased their firing rate during movement (paired t-test, p=0.0042, n=27, df=26). FIGS. 25B-25D are pie chart visualizations of the fraction of ChIs exhibiting >10% increase (red) in firing rate during movement compared to rest (movement-rest)/(movement+rest), >10% decrease (blue), or no change (<10% modulation). FIG. 25B is a pie chart including all ChIs, FIG. 25C shows delta-rhythmic ChIs only, and FIG. 25D shows non-delta ChIs only. FIG. 25E is the same as FIG. 25A, but for SYNs. SYNs, as a population did not change their firing rate (paired t-test; p=0.091, n=25, df=24). FIGS. 25F-25H are the same as FIGS. 25B-25D, but for SYNs. Quantifications are visualized as violin plots with the outer shape representing the data kernel density and box-and-whisker plot (box: interquartile range, whiskers: 1.5× interquartile range, white line: mean). All statistical tests are two-sided. Source data are provided as a Source Data file.

Transitions in movement speed have been shown to critically depend on the striatum and are sensitive to striatal dopamine changes. We thus examined the responses of delta-rhythmic and non-delta rhythmic neurons around movement transitions. An example delta-rhythmic ChI responsive to movement onset is shown in FIG. 24E. We found that delta-rhythmic neurons as a population significantly increased their firing rates around movement onset (FIG. 24F). Non-delta neurons, in contrast, showed no change in firing rate around movement onset (FIG. 24G). By further examining ChI and SYN subgroups within the delta and non-delta populations, we found both delta-rhythmic ChIs and SYNs significantly increased their firing rates around movement onset, while non-delta-rhythmic SYNs did not show movement onset-related firing rate modulations (FIG. 24H, FIGS. 26A-26C), similar to their differential responses during sustained movement periods (FIG. 24B, FIG. 24C). Due to the low number of non-delta ChIs and the limited number of movement transitions, we did not perform this analysis on the non-delta ChI group. Finally, none of the three groups' firing rates changed significantly around movement offset (FIGS. 25D-25F). Together, these results provide direct experimental evidence that delta-rhythmic neurons, both ChIs and SYNs, but not non-delta rhythmic neurons, selectively encode movement onset transitions and sustained movement.

FIGS. 26A-26F show that ChI and SYN delta-rhythmic neurons, but not non-delta neurons, are differentially responsive to movement onset. FIG. 26A shows delta-rhythmic ChI population firing rate at movement onset (n=21). FIG. 26B shows delta-rhythmic SYN population firing rate at movement onset (n=9). FIG. 26C shows non-delta SYN population firing rate at movement onset (n=16). FIG. 26D shows delta-rhythmic ChI population firing rate at movement offset (n=21). FIG. 26E shows delta-rhythmic SYN population firing rate at movement offset (n=9). FIG. 26F shows non-delta SYN population firing rate at movement offset (n=16). All shaded regions around lines represent standard error of mean.

### Example 16—Delta-Rhythmic ChI and SYN Spiking is Differentially Associated with LFP Delta, Beta, and Gamma Rhythms During Movement

Given that delta-rhythmic neurons exhibited stronger spike phase locking to Vm and LFP delta oscillations (FIG. 20K, FIG. 20O), we next examined whether locomotion-related spiking was linked to network delta rhythmicity measured as LFP oscillations. We found that striatal LFP power at delta frequency (2-4 Hz) was elevated during rest, while narrow-band theta (6-8 Hz) and alpha (10-11 Hz) oscillations were elevated during movement (FIG. 23A). Despite the dominant peak in the theta range of LFP power during movement, the spike-LFP PLVu² at delta frequency, but not theta frequency, was significantly increased during locomotion for delta-rhythmic neurons (FIG. 23B), driven specifically by increased PLVu² of delta-rhythmic ChIs (FIG. 23D). This suggests that LFP delta power, while not as striking as LFP theta power, nevertheless showed a movement-specific association to striatal ChIs spiking in the delta frequency range. Neither delta-rhythmic nor non-delta rhythmic SYNs exhibited significant changes in spike-LFP delta PLVu² during movement (FIG. 23C, FIG. 23D). The unique temporal relationship of ChI spiking with LFP delta oscillations highlights an important role of ChIs in influencing striatal network LFP delta rhythmicity.

We next analyzed whether spike-LFP relationships were differentially modulated by movement conditions. We calculated the difference of LFP spectrum power aligned to spikes that occurred during movement versus rest. Delta-rhythmic ChI spiking was associated with significantly increased LFP high-gamma (70-140 Hz), but not beta (20-40 Hz) power, during movement compared to rest. Thus, even though Vm and LFP beta power increased around spikes (FIGS. 22D, 22G, 22H, and 22K), LFP beta oscillations accompanying ChI spiking were not specific to movement. In contrast, delta-rhythmic SYN spiking was associated with a significant decrease in beta power during movement, which started ˜100 ms before the spike and lasted up to ˜200 ms after. The reduction in LFP beta power around SYN spiking is consistent with previous studies reporting overall reductions in LFP beta power during movement. No significant movement-dependent differences in LFP oscillations were detected around spikes of non-delta SYNs.

Finally, we found a selective association of LFP high-gamma power with delta-rhythmic ChI spiking, but not SYN spiking, during movement highlighting a role of ChI spiking in synchronized striatal neuron activation, which is supported by the unique anatomical features of ChIs that form extensive synaptic connections with striatal neurons.

### Example 17—Delta-Rhythmic Neurons Spike in Coordination with Movement Stepping Cycles

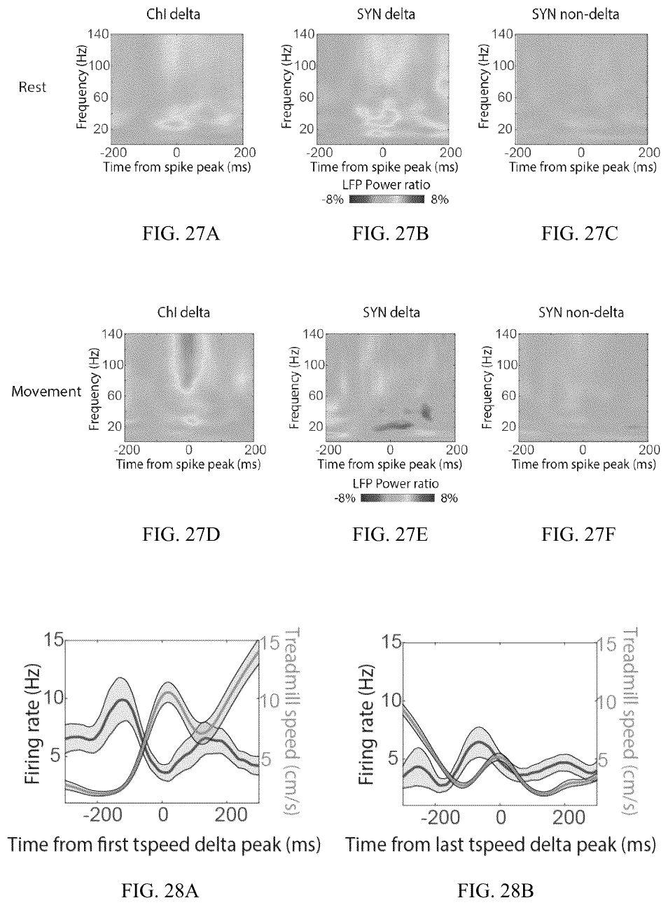

FIGS. 27A-27F illustrate spike-aligned LFP power during rest versus movement. FIGS. 27A-27C show population spike-aligned LFP spectral power during rest for (FIG. 27A) delta-rhythmic ChIs (n=21), (FIG. 27B) delta-rhythmic SYNs (n=9) or (FIG. 27C) non-delta rhythmic SYNs (n=16). FIGS. 27D-27F show population spike-aligned LFP spectral power during locomotion for (FIG. 27D) delta-rhythmic ChIs (n=21), (FIG. 27E) delta-rhythmic SYNs (n=9) or (FIG. 27F) non-delta rhythmic SYNs (n=16). LFP spectral power was normalized to the pre-spike period (−200 to −100 ms).

FIGS. 28A-28B show that the firing rate increases before the first delta cycle peak at locomotion onset and before the last delta cycle peak at locomotion offset. FIG. 28A shows delta-rhythmic neuronal population firing rate aligned to the peak of the first movement-speed delta cycle at locomotion onset (n=31). Green-blue line represents the averaged treadmill speed. FIG. 28B is the same as FIG. 28A, but aligned to the peak of the last movement-speed delta cycle at locomotion offset. All shaded regions around lines represent standard error of mean.

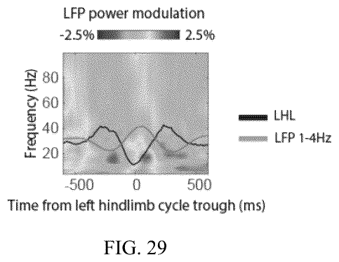

FIG. 29 shows that LFP delta rhythmicity and beta power are coordinated with animal stepping cycles. FIG. 29 shows population LFP spectral power aligned to the trough of animal left hindlimb (LHL) stepping cycle (black), and overlaid with LFP delta cycle (purple).

Stepping movement in humans and mice occur in the delta frequency range, and temporally precise neuronal spiking at delta frequencies in the motor cortex, cerebellum, and thalamus has been linked to rhythmic stepping movements. Intrigued by the frequency similarity between striatal neurons' delta rhythmicity and mouse stepping cycles, we further analyzed mouse stepping during movement. We found that during locomotion periods, movement speed recorded by our treadmill contained delta-rhythmic fluctuations similar to the delta frequency range observed in Vm. To evaluate whether delta-frequency movement-speed fluctuations detected by our treadmill corresponded to stepping cycles, we performed simultaneous video tracking of limb movements and treadmill speed recordings. We found that limb movement was highly coherent with the fluctuations in movement speed at delta-frequencies captured by our treadmill recordings. Specifically, the movement of right and left hind limbs (RHL and LHL, respectively) exhibited strong phase locking to the delta-frequency component of the treadmill speed, with a time-lag between the two hind limbs, reflective of the alternating limb movement patterns within each stepping cycle. Thus, the delta-frequency treadmill speed fluctuations we observed captured animal's delta-rhythmic stepping cycles.

Since spike times were strongly modulated by Vm delta oscillations, we next examined whether delta-rhythmic neurons' spiking activity was coupled to stepping cycles, measured as the delta-frequency component of the treadmill speed. As a population, delta neurons significantly increased their firing rates prior to the peak of treadmill speed delta, as opposed to non-delta neurons. Delta-rhythmic neurons generally spiked in the rising phase (−1.93±0.97 radians, mean±SD) of the delta-frequency component of treadmill speed. Non-delta rhythmic neurons showed a similar phase preference (−2.2±0.98 radians, mean±SD), despite having weak spike phase locking. Indeed, we found that spikes in delta-rhythmic neurons, but not non-delta neurons, exhibited significant phase locking (PLVu²) to the delta-frequency component (2-3 Hz) of mouse locomotion speed during movement. Separating delta-rhythmic ChI and SYN populations revealed that this effect was driven mainly by delta-rhythmic ChIs, but not delta-rhythmic SYNs. Finally, as the instantaneous stepping delta frequencies varied (mean±SD, 2.71±1.23), we examined the delta frequencies below versus above the mean. Most remarkably, delta-rhythmic neurons remained phase locked across different ranges of stepping frequencies.

To further understand the temporal relationship between delta-rhythmic neuron spiking and stepping cycles, we aligned the firing rate to the peak of the first delta cycle within a movement bout. We found that delta-rhythmic neurons significantly increased their firing rate before the first delta peak around movement onset, with the firing rate preceding stepping movement by about 200 ms (FIG. 28A). Similar firing rate changes proceeded the last delta peak around movement offset (FIG. 28B). Thus, stepping-patterned delta-rhythmic neurons' spiking persists throughout the locomotion bouts.

Finally, we found that striatal LFPs were highly coherent to both treadmill speed and left hindlimb movement in the stepping-related delta frequency range. Further, LFP beta (20-40 Hz) and high-gamma (>80 Hz) power were modulated by the stepping cycle (FIG. 29) and the delta-frequency component of the treadmill speed, peaking at a similar phase as the spikes in delta-rhythmic neurons. Thus, striatal LFP beta and high-gamma power are nested within the delta-rhythmic stepping cycle. Stepping-modulated LFP high-gamma power is most likely due to increased neural activities, as high-gamma oscillations have been broadly associated with elevated spike rates. Since spiking and Vm delta rhythmicity in delta-rhythmic neurons are coupled to Vm and LFP beta oscillations regardless of movement conditions, the observed stepping-modulated LFP beta oscillations reflect the synchronization of delta-rhythmic neurons' Vm with stepping cycles. Together, our results demonstrate a prominent role of delta rhythmic striatal neuron spiking and striatal LFPs to influence mouse stepping cycles and organize higher frequency beta and gamma oscillations, providing evidence for a functional role of basal ganglia delta rhythmic dynamics in movement patterning.

### Discussion of Experimental Data of Examples 12-17

Synchronized neural activity at delta frequencies is broadly observed in cortical, subcortical and spinal motor circuits during delta-rhythmic voluntary movement. Network delta rhythms have been proposed to serve as a temporal coordination mechanism to organize higher frequency rhythms and spikin. To understand how cellular dynamics of individual neurons support network delta rhythmicity during locomotion, we performed membrane voltage imaging from genetically-defined striatal cholinergic interneurons (ChIs) and non-specific striatal cells expressing the generic neuronal marker synapsin (SYNs) using the genetically encoded voltage indicator SomArchon. We extracted voltage-dependent SomArchon fluorescence at the cell bodies and analyzed spiking and subthreshold membrane voltage (Vm) at the soma of individual ChIs and SYNs, while mice voluntarily locomoted on a treadmill. We found that many striatal neurons, particularly ChIs, have prominent cellular Vm oscillations and spiking at delta frequencies during both rest and movement. Not only did Vm delta oscillations pace spiking output at beta frequencies, but they were also coupled to LFP delta and beta oscillations, and tightly phase locked to animals' delta-rhythmic stepping cycles. Thus, our study provides direct experimental evidence that delta rhythmicity within single striatal neuron membrane voltage plays an important role in patterning movement.

ChIs, though representing only 1-2% of the striatal cell population, can powerfully modulate striatal networks via their extensive synaptic arborizations that connect broadly throughout the striatal network. A remarkable feature of the ChIs we observed was the sustained Vm oscillations and spike inter-bursting intervals in the delta range (1-4 Hz), during both movement and resting conditions. Given ChIs exhibit similar intrinsic delta-rhythmic Vm depolarizations and spike bursting in vitro without any synaptic inputs, it is likely that intrinsic biophysical mechanisms are critical for shaping the ChI Vm delta rhythms observed here in awake animals, regardless of locomotor conditions. However, ChIs also receive broad cortical and thalamic inputs and many of these inputs exhibit delta-frequency rhythmicity that could contribute to the observed ChI Vm delta oscillations. Furthermore, we also noted the presence of Vm delta oscillations in a subset of non-specific striatal neurons (SYNs), estimated to be primarily SPNs. Since SPNs do not demonstrate an intrinsic resonance frequency preference, the observed cellular delta rhythmicity in SYNs is likely due to synaptic inputs rather than intrinsic biophysical mechanisms.

We also found that ChI spiking is time locked to movement onset, and tracks the animal's stepping cycle in the 1-4 Hz delta frequency range. Studies have reported that certain neurons in the motor cortex, thalamus, and cerebellum have spikes phase locked to LFP delta oscillations, and projections from these areas to the striatum exhibit stepping-cycle dependent activity. Thus, the movement-dependent entrainment of cellular delta rhythms observed in ChIs likely originates from movement-related synaptic inputs.

Not only do ChIs receive extensive dopaminergic inputs from the substantia nigra pars compacta (SNc) and glutamatergic inputs from broad cortical and thalamic regions, but ChIs can directly modulate these inputs via nicotinic acetylcholine receptors (nAchRs) expressed on these input axon terminals. Indeed, fast dopaminergic signaling at SNc axon terminals in the striatum exhibit delta frequency modulation. Since ChI activation can powerfully excite dopaminergic and glutamatergic terminals via nAchRs, it is possible that delta-rhythmic spiking of ChIs paces striatal output through indirect action on these input axonal terminals, in addition to SPNs. SPNs additionally express metabotropic acetylcholine receptors (mAChRs), and activation of ChIs could increase SPN activity, particularly in D2-SPNs that express mainly excitatory mAChR1, which could promote striatal LFP beta oscillations. ChIs have been shown to promote coordination between SPNs, and the elevation of striatal cholinergic tone increases LFP beta and gamma oscillations leading to movement inhibition. We detected an increase in LFP beta power associated with spiking not only in delta-rhythmic ChIs, but also in delta-rhythmic SYNs dominated by SPNs in the absence of movement. It is likely that the LFP beta power around delta-rhythmic SYN spiking is due to direct pacing by ChIs whose spikes are associated with both Vm and LFP beta rhythms.

In addition to rhythmic stepping, other potential rhythmic movements include respiration, sniffing, and whisking which could produce widespread effects on subcortical and cortical motor circuits. The reported respiration frequency in mice is commonly in the theta range (3-10 Hz), particularly during locomotion. Recent studies found no temporal relationship between respiration and stepping cycle suggesting that the Vm delta rhythmicity observed here is unlikely to be linked to the respiration cycle. Whisking, which often occurs at a higher speed of 10 Hz or more, has been shown to be phase coupled to strides during certain behavioral conditions. It is thus possible that some of the Vm delta rhythmicity we observed in striatal neurons is related to other rhythmic movement that is coupled to stepping.

The central pattern generator circuits in the spinal cord are critical for producing autonomous rhythmic motor patterns that usually occur in the delta frequency range. However, the spinal central pattern generator receives substantial supra-spinal control from diverse brainstem circuits, the cerebellum, thalamus, motor cortex, and the basal ganglia. While stepping-cycle modulated activity has been well documented in the cerebellum and motor cortex, the basal ganglia has been traditionally associated with action selection, motor learning, and various locomotion features, rather than motor patterning. Recently, it has been shown that the subthalamic nucleus (STN) of the basal ganglia exhibits stepping cycle dependent activity. Further, striatal neurons code for movement vigor and Parkinsonian patients tend to have gait disturbances including changes in their stepping frequency, suggesting that the basal ganglia may also influence stepping dynamics. In fact, we detected prominent cellular membrane voltage delta rhythmicity in ChIs regardless of movement conditions, which along with ChI's powerful influence on striatal circuitry, suggests that ChIs may not only modulate or pace movement patterns, but provide temporal patterning for a wide variety of sensorimotor and cognitive functions.

Delta-rhythmic population activity in the motor cortex and thalamus has been linked to population dynamics during rhythmic and non-rhythmic motor behaviors. The sustained delta-rhythmic patterning of striatal activity might play a key role in supporting coordination of the cortico-basal ganglia-thalamic circuits during movement, which can interact with spinal circuits via frequency dependent circuit coupling. Finally, striatal neurons are sensitive to various aspects of movement, including movement transitions, speed, direction, and complex moment-to-moment behavioral kinematics. It would be informative to examine how individual striatal neuronal membrane voltage dynamics relate to various movement aspects in addition to movement pattering in future studies.

The functional properties of striatal tonically active neurons (TAN), assumed to largely correspond to cholinergic interneurons, have been extensively examined using extracellular electrophysiology in rodents and primates. However, strong spike delta rhythmicity, as observed here, has not been reported. While we found clear subthreshold delta rhythms in the membrane potential that predicted spike timing, we found no delta rhythmicity in the spike autocorrelogram and only weak delta rhythmicity in the Vm autocorrelogram due to the large cycle or frequency variability within the delta range. This quasi-periodic property is in line with delta-rhythmic pattern generation dynamics reported in the motor cortex.

We found that beta rhythms in both Vm and LFP signals were nested within the delta-rhythm, with a preferred phase similar to the spiking preference of ChIs and SYNs. Stepping-cycle nested LFP beta and gamma power has also been observed in the human STN, suggesting that during locomotion, delta rhythms in the cortico-basal ganglia circuit are coordinated with patterned stepping movement, and beta and gamma oscillations across structures are nested within delta cycles. Further, we observed that neurons with Vm delta oscillations exhibited stronger spike-associated beta power than non-delta neurons, highlighting that striatal delta and beta rhythms are closely linked at the cellular level, which is consistent with reported delta-beta cross-frequency coupling in the motor thalamus and more generally, across the cortico-basal-ganglia-thalamic circuits. Delta-rhythmic neurons, both ChIs and SYNs, had intra-burst firing rates in the beta frequency range, indicating a potential spike-related delta-beta coupling mechanism. However, the observations of increased Vm beta power in the absence of spikes demonstrates that beta rhythms also rely on network mechanisms. Beta power enhancement was limited around particular phases of Vm delta, highlighting the transient nature of beta rhythms. Sustained and exaggerated beta rhythms are a hallmark of Parkinsonian pathology, which likely involves ChIs. Given the close link between delta and beta oscillations in healthy animals, delta rhythmic modulation of beta rhythms in ChIs may be disrupted in the Parkinsonian brain. Future voltage imaging studies in Parkinsonian animal models will provide insights on how loss of dopamine alters ChIs' cellular voltage dynamics and contributes to PD circuit pathophysiology.

LFP beta-band power in the motor system is dynamically regulated during locomotion and exhibits overall suppression during motor execution. Here, we found that delta-rhythmic ChI spiking was not associated with any LFP beta power change between resting and locomotion, but rather was accompanied with an increase in high-gamma oscillations, suggesting synchronized striatal neural activity upon ChI activation. In contrast, delta-rhythmic SYN spiking was coupled to decreased LFP beta during locomotion, demonstrating that SYN cellular dynamics contribute more to striatal LFP beta rhythmicity as LFP beta oscillations were generally suppressed during movement. Furthermore, we also detected significant modulation of LFP beta power by the phase of delta-rhythmic stepping cycles, highlighting that dynamic fluctuations of beta power might support the patterning of movement.

We found 81% of ChIs exhibited delta rhythms, in contrast to 36% of SYNs (primarily SPNs). Here, we cannot delineate whether the delta-rhythmic SYN subset corresponds to a particular type of SPN or to other interneurons. Given the sparsity of ChIs in the striatum (roughly 2%), it is unlikely that ChIs contributed substantially to the delta-rhythmic SYN population, and the majority of delta-rhythmic striatal neurons are not expected to be ChIs. Furthermore, only the delta-rhythmic subset of SYNs exhibited locomotion-dependent firing rate modulations, suggesting that they are part of a locomotion-sensitive striatal circuit. Non-delta-rhythmic neurons exhibited mainly regularly spaced firing patterns, though we noted some neurons showing theta-burst spiking patterns. Future studies delineating other neuron subtypes could reveal the distinct functions of different striatal cell types in locomotion and movement patterning.

### Experimental Procedures—Examples 4-11

### Animal Surgery

All animal procedures were approved by the Boston University Institutional Animal Care and Use Committee. The study included both female and male C57BL/6 mice (n=9) 8-12 weeks at the start of the experiments, obtained from Charles River Laboratories. Mice were first surgically implanted with a sterilized custom imaging window with an attached guide cannula that was assembled prior to surgery. The window/guide assembly was made of a stainless-steel imaging cannula (outer diameter 0.317 cm, inner diameter 0.236 cm, height 2 mm) fitted with a circular coverslip (size 0, outer diameter 3 mm) adhered to the imaging cannula with ultraviolet curable optical adhesive (Norland Products). A guide cannula (26 gauge; No. C135G24; Plastics One) was fixed to the cannula at 45-degree angle to the base of the imaging window. An additional insulated stainless-steel wire was glued to the virus infusion cannula with super glue and protruded from the bottom of the infusion cannula by about 200 m for LFP recordings. Additionally, a small ground pin was inserted near the cerebellum as the ground reference.

A craniotomy about 3 mm in diameter was made over the striatum (anteroposterior +0.5 mm, mediolateral 1.8 mm, dorsoventral −1.6 mm from the brain surface) and a small notch created on the posterior edge of the craniotomy to accommodate the infusion cannula and LFP recording electrode. The overlying cortical striatal tissue was gently aspirated to expose the corpus callosum and visually access the dorsal striatum. Dental cement was applied to fix the imaging window to the skull, and to mount a custom aluminum headplate posterior to the imaging window. Following recovery from surgery (14-21 days post-surgery), one cohort of mice (n=4) were injected with a 1 μl AAV9-Syn-GCaMP7f.WPRE.SV40 virus (titer 6.6×1012 GC ml-1) through the attached guide cannula. The other cohort(n=5) was injected with a 1 μl AAV9-Syn-SomaGCaMP7f.WPRE.SV40 virus (titer 6.6×1012 GC ml-1). After viral infusion (21-28 days), mice were habituated, 3-4 days per week, head fixed on the ball.

### Imaging Data Acquisition

Image acquisition was carried out using a custom microscope outfitted with a scientific complementary metal oxide semiconductor (sCMOS) camera ((ORCA-Flash4.0 LT Digital CMOS camera, No. C11440-42U, Hamamatsu). Mice were positioned under the microscope and imaged during voluntary movement on a spherical treadmill. The custom microscope consisted of a Leica N Plan 10×0.25 PH1 microscope objective lens, a dual-band excitation filter (No. FF01-468/553-25), a dichroic mirror (No. FF493/574-Di01-25×36), a dual-band emission filter (No. FF01-512/630-25; Semrock) and a commercial single light reflex lens focused to infinity as the tube lens (Nikon Zoom-NIKKOR 80-200 mm f/4 AI-s). GCaMP7f and SomaGCaMP7f fluorescence excitation was delivered with a 5 W light-emitting diode (460 nm). The camera coupled to the 10× objective lens yielded an imaging field of view that measured 1.343×1.343 mm²; each pixel corresponded to 1.312×1.312 μm². Image acquisition was accomplished using HC Image Live (Hamamatsu). Image data were stored in dcimg format and converted into tif format. Imaging sessions were recorded simultaneously with movement data and local field potentials as the head-fixed mouse ran voluntarily on a spherical treadmill. For a recording session of 36 minutes, roughly 112 gigabytes of image data were stored across 27 video files (TIFF format).

### Local Field Potential Recordings

The OmniPlex (PLEXON) system recorded the local field potentials at 1 kHz sampling rate. To synchronize movement data, imaging data and LFP recordings, TTL pulses sent by the camera and movement data acquisition along with stimulation triggers were also recorded using this system.

### Movement Data Acquisition

Movement data acquisition was performed on a spherical treadmill. The treadmill consisted of a Styrofoam ball supported by air and a 3D printed plastic housing. Movement was monitored with two computer universal serial bus mouse sensors fixed to the plastic housing encasing the Styrofoam ball at the equator of the ball at an angle of 75 degrees using the Teensy system as described in Romano et al. The x- and y-surface displacement was acquired by each sensor at 20 Hz and sent to the image acquisition computer via RS232 serial link using a custom MATLAB script.

### Sensory Stimulation

Sensory stimulation at a specific frequency was delivered by utilizing a white LED placed at eye level of the mouse and a speaker delivering clicks within ear shot of the mouse. Pulses were delivered (50% duty cycle) via a function generator that was manually operated to deliver the stimulation. The signal was amplified using an amplifier as required for the speaker and the LED. One or two sessions were recorded for each mouse for each frequency. The audiovisual task in a session consisted of five trials of one-minute-long stimulation at the same frequency either 10 Hz or 145 Hz interspaced by five minutes of baseline period (FIG. 1b). There was 6 minutes of baseline period at the start to align all recording systems and additional five minutes baseline period after the last trial giving a total recording session duration of 36 minutes.

### Movement Data Processing

The spherical treadmill was calibrated by pinning two sides of the ball at the equator for physical distance. Linear velocity was calculated as:

$${\hat{y}}_{L} = \frac{{y}_{L -} {y}_{R} cos ( α )}{sin ( α )} \\ v = \sqrt{{\hat{y}}_{L}^{2} + {y}_{R}^{2}} \qquad ( 1 )$$

where α is the angle between the two sensors (75 degrees), v is the instantaneous velocity of the mouse, and y_{L}, y_{R} are the vertical readings from the left and right sensors relative to the surface of the ball as in Romano et al⁸⁷. Any artifacts such as data points larger than 100 cm/s were removed and linear interpolation was applied. Further, a moving average with window size of 20 frames (1 second) was applied to produce smoothed speed trace (movmean in MATLAB). Finally, the speed trace was interpolated to 20 Hz to match the calcium imaging data.

Threshold detection: Utilizing the fact that the variance of speed during low-speed periods would be very low, we can determine the low-speed threshold using moving variance (window size of 40 frames-2 seconds) of the speed trace. All the periods with variance less than 0.1, the mean speed less than 5 cm/s and duration longer than 2 seconds was selected as ‘guessed rest periods’. Low-speed threshold was computed as the mean+2 standard deviations (speeds in guessed rest periods). High-speed threshold was determined as the 50^{th} percentile of the remaining periods. If the above constraints were not satisfied, low-speed threshold was calculated as the 10^{th} percentile and high-speed threshold as the 60^{th} percentile of the speed trace across the recording. We also ensured that the estimated low-speed threshold was reasonable (below 2.5 cm/s). For two sessions in which the mouse was constantly moving, it was set to 2.5 cm/s.

Identification of rest period and movement periods: Rest period was defined as the periods where the speed remained constantly below the low-speed threshold for at least 2 seconds. We applied sigmoid function-based thresholding to use fuzzy logic for thresholds. We first assigned each velocity data point (v) a Fuzzy membership value using a sigmoidal membership function F (equation 2) which scales points that are much higher than the threshold to zeros.

$$F ( v , a , c ) = 1 - \frac{1}{1 + exp ( - a ( v - c ) )} \qquad ( 2 )$$

where v is the speed trace, c is the low-speed threshold, a is set to 0.8 (sigmoid slope). The resulting velocity trace was then smoothed using a moving average filter of 2 seconds and data points greater than 0.45 were ascertained to be rest periods provided the duration was =>2 seconds. We categorized movement periods as periods where the speed trace remained above the low-speed threshold for at least 2 seconds. In a similar manner, the speed trace was passed through the sigmoid function G (equation 3) which scales points that are much higher than the low-speed threshold to 1 and all datapoints greater than 0.55 with a duration of more than 2 seconds were classified as moving periods.

$$G ( v , a , c ) = \frac{1}{1 + exp ( - a ( v - c ) )} \qquad ( 3 )$$

Movement onset transition detection: Movement onset transition was defined as the precise timepoint when the mouse rapidly transitions from rest to movement. Firstly, we computed locally adjusted low-speed thresholds by assigning ones to all points greater than the low-speed threshold and rest of the trace zeros. Then, we looped through all the islands of ones and raised the local low speed threshold by one tenth of the difference between low and high-speed threshold for islands with duration longer than 10 seconds. After two loops of adjustment, the islands of ones are set to be potential motion onset transitions. The exact timepoint was further fine-tuned using the search-box method (search box—a second before and 2 seconds after each onset transition) by applying additional constraints on the identified onsets. The timepoint where acceleration exceeded the threshold (high speed threshold—low speed threshold)/60) within the search box was set to be the onset transition after ensuring that the mouse was in rest for at least 2 seconds prior to the transition and acceleration stayed above the threshold for >250 ms. Visual inspection was performed to confirm the accuracy of the detection.

### Motion Correction

Motion correction was performed with a custom python script⁵³. Reference image was generated for a session by projecting the mean values of every pixel in the first 2047 frames of the recording session. The reference image and each frame of the video underwent a series of image processing steps to enhance the contrast and the character of the image. Firstly the image was high-pass filtered with a Gaussian filter (python SciPy package, ndimage.gaussian_filter, sigma=50) and edges of high intensity areas were enhanced by sharpening the image and consecutively low-pass filtered the image with Gaussian filters at two levels (sigma=2 and 1). Finally, to compensate for potential bleaching, the intensity of each image was normalized by z-scoring. Cross-correlations were calculated between the enhanced reference image and each frame to obtain the displacement between the location of max correlation coefficient and the center of the image. The shift that countered the displacement was applied to the original, unenhanced image to complete the motion correction.

### Trace Extraction and Pre-Processing

Next, we calculated a max-min (maximum fluorescence minus minimum fluorescence) projection image across the whole video stack. This static frame was used to select the regions of interest (ROIs) corresponding to cells using a semi-automated custom MATLAB software. The ROIs were manually selected as circles with a radius of 6 pixels (7.8 μm). This selected sub-region of the image was automatically identified to determine the pixels within that region that correspond to a cell and pixels from each ROI were averaged together spatially to calculate a temporal trace for each neuron. A local background ‘donut’ mask was calculated for each neuron by finding the centroid of the neuron, measuring a circle approximately 5 cell widths in radius (50 pixels). The average pixel intensity in this local background mask was computed with exclusion of areas of all cell ROIs within the background mask. This resulting background trace was further subtracted from the corresponding neuron's trace to remove local fluctuations from scattering in wide-field imaging. Next, the fluorescence traces(Δf/f) were then normalized by the mean, interpolated to exactly 20 Hz and linearly detrended for every neuron. All traces across sessions were visually inspected and specific traces that had artifacts were excluded.

### Calcium Event Detection

We employed a fast activity extraction algorithm from Friedrich et al⁸⁸ that is a generalization of the pool adjacent violators algorithm (PAVA) for isotonic regression for calcium event detection using the hard-shrink method. PAVA infers the most likely event train giving an underlying autoregressive AR1 fluorescence trace. The discretized deconvolved output was further thresholded using a threshold defined to be 2.5 times the standard deviation of the detrended fluorescence trace. The trace was detrended by subtracting the original trace by a smoothed trace using a box-car smooth kernel of 10 frames (500 ms). We performed a manual inspection of event detection in about 100 traces in multiple mice to ensure the accuracy of event detection. We then made the calcium event onsets ones and the rest of the trace zeros which was then used to compute the calcium event onset frequency (density of ones) across periods of interest to compare between GCamp7 and somaGcamp7 (FIG. 12B). In addition, the rising phase of every calcium event was made ones to store a binary trace across the whole recording session. The density of ones in this binary trace for every neuron represents the calcium activity rate across specific periods of interest (FIG. 12A, FIG. 12D). The mean activity rate was 18.39±3.69 frames/min at 20 Hz frame rate. The last minute (35-36 min) of the recording session was excluded for subsequent analyses in all sessions due to photobleaching evident by the decline in event rate as well as in the video.

### Identification of Movement Responsive Neurons

A neuron was categorized as movement responsive if the calcium activity rate was significantly higher in running compared to resting periods in baseline. The classification metric utilized was the difference between the calcium activity rate in the running versus the resting periods (D). To compute the observed value, we concatenated all the rest or run periods separately and summed the activity rate across each concatenated period.

$$D = ( \frac{{\sum }_{run} y}{{\sum }_{run} t} - \frac{{\sum }_{rest} y}{{\sum }_{rest} t} )$$

A shuffled distribution was created by randomly selecting equivalent running and resting periods from the entire baseline period 1000 times and calculating the difference metric in each shuffle. If the observed difference metric was greater than 97.5^{th} percentile of the shuffled distribution, the neuron was classified as a movement responsive neuron. Two sessions that had less than a minute of rest periods in baseline (1200 frames) were excluded from the analysis as it can bias the random sampling.

### Cross Correlation Analysis Between Population Activity Rate and Movement

We estimated the correlation coefficient between the speed trace and the summed activity density rate across movement responsive neurons at different time lags using the python package scipy.correlate to cross-correlate two N-dimensional arrays. The time point corresponding to the peak correlation coefficient was estimated as the temporal lag. Sessions with reasonable lags between 0 and 1 s were included in the analysis. This was further verified by shuffling the summed activity rate by random lags and calculating the correlation coefficient with the speed trace to build a shuffle distribution for every session. The correlation at the estimated lag was significantly greater than the threshold (95^{th}percentile) of the shuffled distribution.

### Detection of Stimulation Responsive Cells

We identified stimulation responsive neurons by comparing the mean activity rate in stimulation versus baseline in the same locomotion state (rest or run) to discern the sensory response from the motor response. The shuffled distribution was built using 1000 shuffles where we computed the mean activity rate in randomly chosen baseline period in a particular locomotion state in each shuffle. If the observed value during stimulation period is significantly higher than the 97.5^{th} percentile of the shuffled baseline distribution, the neuron was considered activated by sensory stimulation and if the activity rate is lower than 2.5^{th} percentile of the shuffled distribution, the neuron was deemed inhibited by stimulation during the given locomotion state. Sessions with less than one minute of stimulation period or if the duration of baseline period was less than twice the duration of the corresponding stimulation period were excluded for this analysis.

### Neuron Pairwise Correlation Analysis

Correlation coefficient was calculated as the Pearson correlation coefficient of a neuron pair's calcium activity traces. We compared the median pairwise baseline Pearson correlation coefficient between the traces across neuron pairs across specific periods of interest. A shuffling procedure was used to determine whether a neuron pair was significantly correlated (significantly higher correlation than expected by random overlap). Random delays were assigned between two calcium activity traces by circularly shifting a trace by a random number of timepoints and we computed the correlation coefficient between these shifted traces in each shuffle. This process was repeated 1000 times to build a shuffle distribution for every neuron pair and if the observed value was greater than the threshold (97.5th percentile of the distribution), the neuron pairs were deemed ‘correlated pairs’. Negative correlations were small in magnitude and examining individual neuron pairs showed that it is due to the sparseness of calcium events. Therefore, the negatively correlated pairs (<2.5^{th} percentile) were excluded for subsequent analyses. We estimated the percentage of correlated neuron pairs as a fraction of all neuron pairs in a session. To study the modulation of correlation during specific periods of interest, we concatenated activity traces in those periods and computed the median correlation coefficient across the correlated neuron pairs. We evaluated the impact of event rate on the identification of correlated pairs by examining the threshold (97.5th percentile), median (50th percentile) and mean of the shuffled distribution (assigning random lags) of correlation coefficients of given neuron pair. We found that the threshold and mean of the shuffled correlation values are not altered by changes in event rate (FIGS. 15A-15B). On the other hand, the median across shuffles increases with high event rates (FIG. 15C). Thus, we subtracted the median correlation coefficient across 1000 shuffles from the observed correlation coefficient for every neuron pair to correct for any bias due to activity rate and hence compute the normalized correlation coefficient as the pairwise connectivity strength.

### LFP Power Spectrum

LFP artifacts were removed when data point was beyond the range of mean±5 standard deviations across the recording. Removed periods were only a few seconds long with maximum duration of one minute. Further analysis such as power and spectral density computations exclude these periods. One LFP recording was removed due to large artifacts. Wavelet transformation with Pywavelets package was performed using the Complex Morlet wavelet named ‘cmor14.0-1.5’ with bandwidth 14, center frequency 1.5, and scaled between 15 and 5000 resulting in frequency bins between 1 Hz and 100 Hz. To compare the power between running and resting periods in baseline for specific frequency ranges, we combined 10 Hz and 145 Hz recording sessions. Further, the power spectrogram was normalized at each time point by the total power across all frequencies at that time point to probe the relative distribution of power at frequency bands of interest. Normalized LFP power was examined at different frequency bands such as theta (6-8 Hz), gamma (45-80 Hz) and beta (15-30 Hz) by averaging the power across the frequency bins within these frequency bands.

### Entrainment Analyses Calculation

We applied the Hilbert transform method to find the instantaneous power from the analytical signal and compute power modulation in stimulation frequency bands during sensory stimulation versus baseline across trials. The speaker produced an artifact in the band for high frequency stimulation for certain sessions. To check for artifacts, we aligned LFP to every stimulation trigger and visually inspected the stimulation triggered average LFP waveform (evoked response) to visualize and inspect if it looked biological or stimulation evoked artifact/waveform. Further, we also plotted the spectrogram for every trial to confirm as artifacts lead to a substantial increase in power which is not biological. To further confirm entrainment and obtain a measure of phase locking between stimulation train and LFP, we calculated the phase locking value (PLV), defined as:

$$PLV ( f ) = \vert \frac{1}{N} \vert {\sum }_{N} {e}^{i φ ( f , n )}$$

where the phase of a given frequency f was obtained from the Hilbert Transform of the stimulation pulse train. Hilbert Transform was performed on filtered trace using a Butterworth filter (filter order=2) around 10 Hz. The PLV was calculated across sessions with exclusion of trials with detected artifacts.

### Treadmill Speed Delta Component Analysis

Power spectral analysis of the treadmill speed trace showed a dominant component around 3-4 Hz consistent with rhythms visually observed in the speed trace. The speed trace was band pass filtered using a Butterworth filter in broad delta frequency range (2-4 Hz) to detect peaks. LFP spectrograms were calculated with the FieldTrip toolbox⁹⁰ (https://www.fieldtriptoolbox.org/) for Matlab using the wavelet method (Morlet wavelets). Further, we aligned the LFP spectrogram to these peaks and computed power which was normalized to the mean power across the delta-peak triggered window (FIG. 21). Further, we averaged across all delta peaks and recording sessions to generate the mean delta triggered LFP spectrogram.

### Phase Locking Analyses

To compute the phase-locking of calcium event onsets to treadmill speed, we first extracted the instantaneous phase of the delta-rhythmic treadmill speed signals. This was done by bandpass filtering the treadmill speed traces (3-4 Hz) using a Butterworth filter and Hilbert Transform was applied to obtain the analytical signal from which the instantaneous phase could be obtained. Phase locking between calcium event onsets and the delta frequency component of treadmill speed (FIGS. 5A-5F) were computed. We first calculated the phase-locking value (PLV) as in our previous study⁸:

$$PLV ( f ) = \vert \frac{1}{N} \vert {\sum }_{N} {e}^{i φ ( f , n )}$$

where f is the frequency and N is the total number of events. The phase ϕ was obtained from the Hilbert Transform analytical signal. To examine phase locking of calcium events to delta-filtered traces in neurons with generally low and varied event rates, we utilized an unbiased PLV estimator (PLVu²) that accounts for the number of events. Since PLV is biased with low number of events, we only included neurons with at least 20 events. Further, the PLV value was adjusted using the following equation and termed as unbiased phase locking value (PLVu²) as in our previous study⁸:

$${PLVu}^{2} ( f ) = \frac{1}{N - 1} ( {PLV ( f )}^{2} \times N - 1 )$$

Running periods were selectively used for these analyses since the delta frequency component in speed, reflecting the stepping cycles, was predominant only during movement. PLVu2 was computed by concatenating across neurons. We compared the phase locking measures during running periods in stimulation versus baseline periods to study the influence of stimulation. Polar histograms were created by plotting the preferred phase (circular mean value) of each neuron relative to the delta-filtered treadmill speed signal. For testing for statistical significance, we randomized the event times and recomputed the PLVu² for each neuron. We then performed a Wilcoxon signed rank test or Paired t-test between the true and randomized PLVu² values. The phase locking values between LFP and Ca events and the statistical testing were also computed using the procedure outlined above using the adjusted PLVu²⁻Ca event-LFP phase locking. In case of phase locking analysis with LFP, the traces were resampled to 1000 Hz to match the LFP sampling frequency.

### Cross-Frequency Coupling (CFC)

To assess phase-amplitude CFC between the delta-phase of the ball-tracking motion signal, believed to correspond to stepping, and either beta (15-30 Hz) or gamma-band (40-100 Hz) amplitude, we followed these steps:
(1) To extract the delta phase, we filtered the ball motion tracking signal within the delta frequency range using a Butterworth filter (filtered order=2) and then applied the Hilbert transform. From the resulting analytical signal, we derived the instantaneous phase within the delta frequency range.
(2) For amplitude extraction, we filtered (filtered order=2) the LFP signal within either the beta frequency range or gamma range and applied the Hilbert transform. Subsequently, we obtained the instantaneous amplitude from the analytical signal.
(3) To calculate the CFC, we once again employed the Hilbert Transform, this time on the instantaneous amplitude to derive the instantaneous phase of beta or gamma amplitude fluctuations. Thus, two phase vectors were derived: one representing the delta phase of the ball motion and the other representing the phase of beta or gamma amplitude fluctuations.
(4)To obtain the CFC value, we computed the phase-locking value (PLV) between these phase vectors. The PLV is a linear measure, yielding a value of 1 if the phase differences remain constant and 0 if the phase differences are uniformly circularly distributed.

### Statistical Analyses

Wilcoxon signed rank test was applied for pairwise analyses of a metric between baseline and stimulation to compare the medians between the two distributions. We used significance level α=0.05 for all statistical tests. All statistics using Wilcoxon signed rank test were done on recording session or trial level. Mixed effect models were utilized to test for the influence of motion and stimulation on activity rates of movement responsive neurons across sessions (MATLAB function fitglme). We used Poisson distribution with log link function in the model. The fixed effects in the model were motion (rest and run), stimulation (on and off) and an interaction term (stimulation×motion) while the random effect was the mouse ID resulting in model structure:

$$Y \sim Motion + Stim + Motion \times Stim + ( 1 \vert Mouse ID )$$

where Y is the activity rate. We computed the R² for the model and tested the coefficients for significance (10 Hz: R²=0.59, n=2189; 145 Hz: R²=0.49, n=2041). We also compared the fit of the model to an intercept-only model, motion-only model, motion and stimulation without interaction model using measures such as log likelihood and Akaike information criterion (AIC). Higher AIC with lower log likelihood indicates better fit of the model. We found that the chosen model exhibited highest AIC and lowest log likelihood compared to other models tested. Motion was identified as a significant factor in the absence of sensory stimulation (mixed effect model, 10 Hz: pₐₚₚᵣₒₓ=0, 145 Hz: pₐₚₚᵣₒₓ=0) which is as expected because movement responsive neurons show a robust increase in activity rate during sustained locomotion.

### Experimental Procedures—Examples 12-17

### Mouse Surgery

All animal experiments were performed in accordance with the National Institute of Health Guide for Laboratory Animals and approved by the Boston University Institutional Animal Care and Use and Biosafety Committees. Same sex mice from the same litters were generally housed together prior to surgery and single-housed post-surgery. Enrichment in the form of Igloos and running wheels was provided. Animal facilities were maintained around 70° F. and 50% humidity and were kept on a 12 hr light/dark cycle.

We used a total of 12 adult mice including 5 ChAT-Cre mice (3 males, 2 females; ChAT-cre; Tg(Chat-cre)GM24Gsat/Mmucd, MMRRC_017269-UCD, NIH MMRRC) injected with FLEX-SomArchon, 3 ChAT-Cre mice (2 females, 1 male) injected with syn-SomArchon, and 4 ChAT-tdT mice (4 females; crossed between ChAT-Cre and the tdT mouse line: B6.Cg-Gt(ROSA)26Sor^{tm14(CAG-tdTomato)Hze}/J from The Jackson Laboratory) injected with syn-SomArchon. Mice were 10-22 weeks old at the start of the study. Data from male and female mice were pooled for the analysis. No statistical method was used to predetermine sample size. Striatal window surgeries were performed similar to those described previously^{35,41,46,63,64}. Custom imaging windows consisted of a stainless steel cannula (OD: 3.17 mm, ID: 2.36 mm, 1 or 2 mm height, AmazonSupply, B004TUE45E) with a circular coverslip (#0, OD: 3 mm, Deckglaser Cover Glasses, Warner Instruments Inc., 64-0726 (CS-3R-0)) adhered to the bottom using a UV curable glue (Norland Products Inc., Norland Optical Adhesive 60, P/N 6001). We glued a virus infusion cannula (26G, PlasticsOne Inc., C135GS-4/SPC), and an LFP electrode made of stainless steel wire (Diameter: 130 μm, PlasticsOne Inc., 005SW-30S, 7N003736501F) to the side of the imaging window using super glue (Henkel Corp., Loctite 414 and Loctite 713). The LFP electrode protruded from the bottom of the imaging window by about 200 μm.

A craniotomy ˜3 mm in diameter was made over the striatum (AP: +0.5, ML: −1.8). A small notch was made on the posterior edge of the craniotomy to accommodate the infusion cannula and LFP recording electrode. The overlying cortex was gently aspirated using the corpus callosum as a landmark. The corpus callosum was then carefully thinned to expose the dorsal striatum. The imaging window was positioned in the craniotomy, and Kwik sil adhesive (World Precision Instruments LLC, KWIK-SIL) was applied around the edges of the imaging window to hold it in place and to prevent any dental cement from touching the brain. Three small screws (J. J. Morris Co., F000CE094) were screwed into the skull to further anchor the imaging window to the skull, and a small ground pin was inserted into the posterior part of the brain near the lambda suture as a ground reference for LFP recordings. Dental cement was then gently applied to affix the imaging window to the exposed skull, and to mount an aluminum headbar posterior to the imaging window.

AAV virus injection occurred either one week prior to window implantation surgery, or through the virus infusion cannula after window implantation surgery. All AAVs were produced by the University of North Carolina Chapel Hill Vector Core. Sequences of the proteins used in this study are available at Genbank at the following accession code: SomArchon MN091368. Plasmids for the viruses used in this study and their sequences are available at Addgene.org (pAAV-CAG-FLEX-SomArchon, Addgene #: 126943; pAAV-synSomArchon, Addgene #: 126941). AAV-Syn-SomArchon (5.9e12 genome copies (GC)/ml) or AAV-CAG-FLEX-SomArchon (6.3e12 GC/ml) was injected into the dorsal striatum (AP:+0.5, ML:−1.8, DV:−2.2, 1 uL virus). Viral injection occurred at 50-100 nL/min. In mice where AAV was injected during surgery, AAVs were infused with a 10 uL syringe (NANOFIL, World Precision Instruments LLC) fitted with a 33 gauge needle (World Precision Instruments LLC, NF33BL) and controlled by a microinfusion pump (World Precision Instruments LLC, UltraMicroPump3-4). In mice where AAV was infused after window implantation, 1 uL of AAV virus was infused through an internal infusion cannula (33G, PlasticsOne Inc., C315IS-4/SPC) connected to a microinfusion pump (World Precision Instruments LLC, UltraMicroPump3-4), approximately one week after the window implantation surgery. The internal infusion cannula was left in place for 10 minutes following injection to facilitate viral spread. Mice were awake and head-fixed throughout the injection period.

All mice were treated with buprenex (0.1 mg/kg) for 48 hours following surgery or with sustained release (SR) buprenorphine (3.25 mg/kg) at the beginning of surgery and single-housed to prevent any damage to the headbar or window implant.

### Head-Fixed Voluntary Movement Experiments

All voluntary movement experiments were performed while awake. Head-fixed mice were allowed to freely navigate using a treadmill made of a Styrofoam ball as previously described, but pinned along the center axis to restrict free movement to forward or backward motion only (no lateral movement). Movement was tracked using two computer mouse sensors positioned roughly ±75 degrees from center along the equator of the ball. In order to determine the mouse movement speed, the pinned ball was rotated vertically to calibrate sensor displacement. All motion sensor displacement data was acquired at 20 Hz on an Arduino Teensy board and synthesized using a custom Matlab script. The timing of each motion sensor displacement data point was also recorded using the OmniPlex system (PLEXON Inc.) for offline synchronization with optical voltage recordings.

All mice were habituated on the treadmill for at least three days, at least 20 minutes per day, prior to image acquisition. During optical imaging, mice were imaged while freely navigating the treadmill.

### Local Field Potential Recording

LFPs were recorded using OmniPlex (PLEXON Inc.) at a 1 kHz sampling rate. To synchronize voltage imaging and LFP recordings during offline data analysis, the OmniPlex system also recorded the TTL pulses that were sent by the sCMOS camera at the onset of each image frame.

### SomArchon Voltage Imaging

All optical recordings were acquired on a custom widefield fluorescence microscope equipped with a Hamamatsu ORCA Fusion Digital CMOS camera (Hamamatsu Photonics K.K., C14440-20UP), 10× NA0.25 LMPlanFI air objective (Olympus Corp.), 40×NA0.8 LUMPlanFI/IR water immersion objective (Olympus Corp.), 470 nm LED (ThorLabs Inc., M470L3), a 140 mW near-infrared 637 nm laser (Coherent Obis 637-140X), a green filter set (Olympus,OCT49002BX3) with a 470/25 nm bandpass excitation filter, a 495 nm dichroic, and a 525/50 nm bandpass emission filter, and a near infrared filter set with a 635 nm laser dichroic filter, and a 664 nm long pass emission filter (Olympus,OCT49006BX3). The near-infrared laser illuminated a circular area of ˜70 μm in diameter, with a field of view (FOV) size of ˜50 μm×70 μm under the 40× objective lens. Our previous computational modeling study showed that fluorescence crosstalk is negligible when nearby fluorescent neurons were >30 um away laterally, regardless of axial distance. We thus primarily recorded from FOVs with only one or two neurons, to minimize potential fluorescence crosstalk. A mechanical shutter (Newport Corporation, model 76995) was positioned in the laser path to control the timing of illumination over the imaging window.

For each FOV, we first collected the GFP fluorescence of SomArchon-GFP fusion protein in the green channel (λₑₓ=470 nm) at 1024×1024 pixels with 2×2 binning to capture cell morphology. SomArchon optical voltage recordings were then performed in the near infrared channel (λₑₓ=637 nm) with 2×2 binning. Optical recordings were acquired at ˜833 Hz with HCImage Live (Hamamatsu Photonics K.K.). HC Image Live data were stored as DCAM image files (DCIMG) and analyzed offline in Fiji/ImageJ and MATLAB (Mathworks Inc.). To synchronize optical recordings with LFP recordings, the camera sent out a TTL pulse to the OmniPlex system (PLEXON Inc.) at the onset of imaging and after each acquired frame. During each recording, we first performed a test trial to ensure spiking activity was present in the putative neuron soma before running the full recording protocol of up to 10 trials, 12 seconds per trial with an inter-trial interval of at least 30 seconds in duration to reduce photobleaching.

For each recorded neuron, SomArchon fluorescence traces were first visually inspected to ensure a lack of significant image motion and reasonable signal-to-noise level based on spike appearance before further analysis. Neurons that passed this visual inspection stage were further analyzed, resulting in 27 ChIs (mean±standard deviation: 4.5±2.1 ChIs per mouse, n=6 mice) and 25 SYNs (3.6±3.5 SYNs per mouse, n=7 mice).

### Voltage Imaging Data Motion Correction & ROI Identification

Motion correction was performed with a custom Matlab script. SomArchon fluorescence images were first motion corrected using a pairwise rigid motion correction algorithm as described previously. Briefly, the displacement of each image was computed by identifying the max cross-correlation coefficient between each image and the reference image. Our recordings consisted of multiple 12-second-long trials. Each video file corresponding to one trial was first concatenated into a multi-trial data matrix, after which the motion correction algorithm was applied. Since the laser illumination area was about 70 μm in diameter, a rectangular window large enough to cover the entire neuron across all frames was selected manually for motion correction. The window selection was chosen to avoid dark regions of the image and to include regions that had distinguishable cell-like contrasts to facilitate comparison with reference image. Each trial was first motion corrected individually. We then corrected image shifts across trials by referencing all trials to the first trial. The motion-corrected image sequences were then used for subsequent manual ROI neuron identification using the drawPolygon function in Matlab. SomArchon fluorescence traces for each ROI were then extracted from the motion-corrected image sequences. The optically-recorded voltage traces for each ROI were generated from the motion-corrected image sequences and were then used for analyses. Due to photobleaching, the quality of SomArchon recording decreases over time. We therefore excluded trials (10.95%) where the spike-to-baseline ratio (SBR, see below) dropped below a SBR average of 5. Further, due to sensitivity of Vm traces to image motion, for Vm analysis we excluded time periods (26.26%) for each recording where there was image motion of >0.065 μm/ms calculated as the combined rectified X-Y derivative of the image motion.”

### SomArchon Fluorescence Detrending and Spike Detection

All optically-recorded SomArchon traces reported in the manuscript were corrected for photobleaching by subtracting the smoothed trace using the Matlab function fastsmooth (https://www.mathworks.com/matlabcentral/fileexchange/19998-fast-smoothing-function). Spike detection was performed similar to that described previously in Xiao et al. To estimate baseline fluorescence, we first averaged the fluorescence trace using a moving window of 100 frames to obtain the “Smoothed Trace” (ST). We then removed potential spike contributions to the baseline by replacing fluorescence values above the ST with the corresponding ST values, which resulted in a spike-removed trace including only the subthreshold baseline fluctuation. To identify spikes, SomArchon fluorescence traces were high-pass filtered (>120 Hz), and spikes were identified as fluorescence increases greater than 4 standard deviations above baseline subthreshold fluctuations.

### Signal-to-Baseline Ratio (SBR) Calculation

To calculate SBR, we first determined the spike amplitude by calculating the difference between the lowest and peak spike fluorescence value within three data points prior to the spike. We then divided the spike amplitude by the standard deviation of the Vm across the entire recording duration.

### Subthreshold Membrane Voltage (Vm) Traces

To obtain subthreshold membrane voltage (Vm) traces, we removed three data points around the peak of each detected spike from the non-filtered SomArchon dF/F trace and interpolated the missing data points using the surrounding data points (n=+/−3 points).

### SomArchon Voltage Imaging, LFP, and Animal Locomotion Data Alignment

Voltage imaging data, LFP data, and animal locomotion data were aligned to the camera start trigger (first frame) of each trial and interpolated to a frame rate of 1 kHz to match that of the LFP recordings. Subsequent analyses were performed on these aligned and interpolated data.

### Inter-Spike Interval (ISI) Calculation

Inter-spike intervals were calculated as the time between identified spikes in milliseconds. For a given spike, we computed the time difference between the previous spike (ISI n) and the following spike (ISI n+1). The two-dimensional space of ISI n and ISI n+1 represent the so-called ISI return map. Plotting the ISI return map, on a logarithmic scale, helped to visualize the bursty and delta-rhythmic spiking patterns of many neurons, particularly striatal ChI neurons.

### Neuron Classification Based on ISI Profile

We observed neurons with distinct ISI structures. Particularly, a subset of neurons exhibited strong delta-rhythmic (2-3 Hz) spiking patterns. Other neurons had more regular spiking patterns. We also observed other spiking patterns, including theta bursting neuron (5-8 Hz) and fast-spiking neurons, but due to their low numbers, they were not analyzed separately. All neurons that did not exhibit a clear delta-rhythmic spiking component constituted the non-delta group. To capture the different types of single neuron ISI curves systematically, we computed an ISI ratio (A-B)/(A+B) defined as: the number of spikes with ISIs of 300-700 ms (1.3-3.3 Hz) being A and the number of spikes with ISIs of 80-200 ms (5-12.5 Hz) being B. This ratio captured neurons with prominent ISI delta peaks. To categorize each neuron as delta-rhythmic versus non-delta, we first manually inspected each neuron's ISI return map, and determined that delta rhythmic ISIs could be well separated from other neurons using the ISI ratio 0. We thus defined neurons as delta-rhythmic which had an ISI ratio of at least 0 or more. Most delta-rhythmic neurons spiked several times per cycle, however, a few neurons only had one spike per cycle.

### Spike Phase-Locking Computation

To quantify how consistent spikes occurred relative to the oscillation phase, we first calculated the phase-locking value (PLV) defined as:

$$PLV ( f ) = \vert \frac{1}{N} \vert {\sum }_{N} {e}^{i ∅ ( f , n )}$$

where f is frequency and N is the total number of spikes. The phase <| was obtained from the complex wavelet spectrum.

Since PLV is not independent of the number of spikes considered and tends to inflate with low numbers of spikes, we only included neurons that had at least 10 spikes for spike-PLV analysis. Further, we adjusted the PLV value using the following equation to account for any potential differences in the number of spikes between groups of neurons, which we term here as unbiased phase locking value (PLVu²):

$${PLVu}^{2} ( f ) = \frac{1}{N - 1} ( {PLV ( f )}^{2} \times N - 1 )$$

where N is the number of spike occurrences and f is frequency. The unbiased PLVu² corresponds, at larger N, to the default PLV².

### Spike-Triggered Vm and LFP Spectrograms

All spectrograms were calculated with the FieldTrip toolbox (https://www.fieldtriptoolbox.org/) for Matlab using the wavelet method (morlet wavelets). Spectrograms for each neuron were aligned to each spike and the time window 250 ms before and after each spike was averaged to create a spike-triggered spectrogram per neuron. Population spectrograms were created by averaging across the spike-triggered (or delta cycle peak-triggered) spectrograms for delta-rhythmic neurons, non-delta rhythmic neurons, SYN or ChI populations, respectively. For FIGS. 27A-27B spectrograms were aligned only to spikes that occurred in rest or movement periods respectively. Spectrograms were normalized to the averaged pre-spike power between −200 to −100 ms before the trigger.

### Spike Raster Plot

For the example neuron in FIG. 24E, the spike timing was plotted per trial aligned to movement onset. The average spike rate was then calculated across all onsets for the neuron.

### Autocorrelograms and FWHM Calculation

For FIGS. 20E-20G, we computed the spike train and Vm autocorrelograms using the xcorr matlab function. Shown are time lags from +3 ms to +2500 ms, as at time 0 the plot is dominated by the perfect autocorrelation peak (correlation with itself). To compute the instantaneous frequency distribution (FIG. 20F, 20G), we filtered the Vm signals in the broad delta frequency range (1-6 Hz) to capture most of the frequency variation. We then applied a Hilbert Transform to obtain the analytical signal from which we derived the instantaneous frequency (IF). To obtain IF estimates, we unwrapped and smoothed the instantaneous phases with a savitzky-golay filter with polyorder=2 and framesize=201 ms and differentiation order=1. For the peak frequency estimate in FIGS. 20H, 20I, for each neuron, we selected the frequency with maximum probability of occurrence. To estimate the full-width-at-half-maximum (FWHM) for each neuron, we quantified the lowest and highest frequency that fulfilled the condition of being half the maximum probability of the instantaneous frequency distribution.

### Definition of Movement Periods and Transition Points

To identify movement bouts, animals' movement data was first smoothed using a 1.5 Hz low pass Butterworth filter to define the transitions more robustly. We defined low speed (“rest”) periods as intervals where the speed was below ≤5 cm/s and high speed (“movement”) periods as intervals where the speed was above ≥5 cm/s. A movement transition was defined as the point at which a pre-defined rest period and movement period intersect, where α rest-to-movement transition (or “onset”) was identified by a movement period directly following a rest period, and vice versa for a movement-to-rest transition (or “offset”) where α rest period directly followed a movement period. In FIG. 24B and FIG. 24C, the firing rates during these rest and movement periods were compared. For each neuron, we calculated the average spike rate per second in rest periods (total spikes/number of rest frames x1000) and the average spike rate per second in movement periods across trials. The difference between the average spike rates in rest and movement periods was used as the metric. To determine if a neuron was responsive to sustained periods of movement, we built a basal distribution of the differences using shuffling. We randomly chose the same number of frames as the total rest frames in each trial and calculated the spike rate across these randomly selected frames and finally took an average across trials for every neuron. This was repeated to calculate the average spike rate in random movement periods as well. The difference between these random spike rates was calculated and this procedure was repeated 1000 times. If the observed difference for a neuron was beyond 97.5^{th} percentile of the distribution, then the neuron was classified to exhibit a significant increased response during movement. On the other hand, if the observed difference was less than 2.5^{th} percentile of the distribution, the neuron was classified to exhibit a significant decreased response during movement.

### Movement-Triggered Spike Rate

For each neuron, average spike rate across one second time windows (500 ms before and after each transition) was calculated in 100 ms intervals and averaged across all onset or offset transitions per neuron, respectively. To determine the statistical significance of these movement-triggered spike rates compared to chance, shuffling was used. For each neuron, the same number of points as the number of onset transition points were randomly selected. One second time windows (500 ms before and after each transition) around these random transition points were concatenated across all transitions. The windows across neurons were concatenated and an average across these windows was calculated. Then, the average spike rate was calculated as the moving mean over a sliding window of 100 ms. This shuffling was done 1000 times to build a basal distribution of average spike rates. The actual metric from observation was also calculated in the same manner using the identified onset transition points. If the calculated movement triggered spike rate exceeded the 97.5^{th} percentile or was lower than the 2.5^{th} percentile (equivalent to being beyond 2 standard deviations), then the spike rate was deemed to be significant and p value was calculated. Significance was determined if a spike rate exceeded+2 standard deviations above or below the shuffled chance value.

### Animal Limb Motion Tracking

To correlate animal's limb positions with treadmill speed, we performed simultaneous video recordings of animal's limbs while recording treadmill movement speed, as described earlier (Head-fixed voluntary movement experiments). Video recordings of the limbs were performed with two Logitech C910 webcams, with each camera capturing the hind limb and the front limb on each side of the body at 120 Hz. Videos were collected with the open-source software OBS studio (https://obsproject.com/), and offline trimmed and cropped using the FFmpeg software (https://ffmpeg.org/). To synchronize video recordings and treadmill speed recordings, an LED light was activated at the start of each recording via TTL pulses generated by the OmniPlex system (PLEXON). As was described for voltage imaging experiments, the OmniPlex system also recorded the time stamps from each movement sensor used to monitor treadmill speed. We then identified the video frame where the LED light was first detected and used that frame to align limb videos to treadmill speed recordings.

Limb videos were offline analyzed using DeepLabCut⁶⁷. Specifically, we first used DeepLabCut to identify 20 representative frames per training video using the built-in k-means clustering algorithm. We manually marked the hind limb and the front limb positions on these representative frames as ground truth frames. We then trained a deep neural network using ground truth frames from 13 videos from 3 mice recorded. Ground truth frames were iteratively improved by refining the limb positions on frames that were identified as outliers by DeepLabCut during the training process. The tracking performance of the final DeepLabCut deep neural network was evaluated by manual visual inspection and quantified by the likelihood value for each frame. The mean average Euclidean error (MAE, proportional to the average root mean square error) was calculated between the predicted and actual user-labeled positions to evaluate the performance of the network. The MAE for the training set (95% of the manually labeled frames) was 2.36 pixels, and for the test set (5% of the manually labeled frames) was 5.21 for all frames or 4.52 for frames with a likelihood greater than 0.6, compared to the average limb area of 1353 pixels (averaged over 48 frames identified in ImageJ).

The limb coordinates (x,y) were used to calculate the relative position of each limb as the resultant vector length. Once treadmill speed traces were aligned with limb positions, coherence between the two signals (treadmill speed, left hindlimb) was computed using the coherence function from the fieldtrip toolbox (http://fieldtriptoolbox.org). Periods of limb locomotion were defined as when the amplitude envelope of the left hindlimb signal derivative exceeded >0.1. To obtain a randomized distribution, we first flipped the trial time of the treadmill speed signals, which removes the temporal correspondence between the treadmill speed trace and the left hindlimb position. We then shuffled the labels of the true and randomized data and calculated the coherence values to obtain a shuffled null distribution, which is then used to assess the likelihood of the true coherence value relative to the shuffled null distribution (p<0.05). Average left and right hind limb positions were then plotted relative to peaks in the delta-filtered treadmill speed traces.

### Treadmill Speed Delta Phase Analysis

In the unprocessed treadmill speed traces, >1 Hz rhythmic fluctuations can be observed during animal movement (e.g. see FIG. 24A). Spectral analysis showed that the rhythmicity has a peak in the delta range with a dominant peak around 2-3 Hz. To compute the phase-locking of striatal neuron spiking to treadmill speed, as well as for analysis of treadmill delta-peak triggered Vm power, we first extracted the instantaneous phase of the delta-rhythmic treadmill speed signals. If not otherwise mentioned, we bandpass filtered the treadmill speed data between 1-4 Hz with a butterworth filter. To obtain the instantaneous phase, we applied the Hilbert Transform providing the analytical signal from which the phase could be derived. Phase locking between spiking or LFP and specific frequencies of treadmill speed were then conducted as described in Spike-phase locking strength calculation above. Data was separated between movement and resting periods. Spike-phase-locking analysis was performed to delta-filtered treadmill speed at 1-2.6 Hz or 2.8-6 Hz.

Polar histograms were created by plotting the preferred spiking phase of each neuron relative to the delta-filtered treadmill speed signal. Some polar histograms depict the preferred phase of beta (20-40 Hz) or high gamma (70-100 Hz) power relative to the delta-frequency filtered treadmill speed signals. Each dot represents an LFP recording session and the shaded regions across all plots represent the preferred phase (phase with the highest mean power). The Omnibus test for non-uniformity was performed to test whether the distribution of preferred phases differed from uniformity as expected from a random process.

The LFP spectrogram was created similarly as described in Spike-triggered Vm andLFP spectrograms above, but instead triggered to the peaks of delta-filtered treadmill speed traces. Power was normalized to the mean power across the delta-peak triggered window, averaged across all delta peaks and recording sessions. Similarly, the spectrogram in FIG. 29 was triggered to left-hind-limb (LHL) cycle trough and power was normalized to the mean power across the window and averaged across LHL cycles and recordings.

### Histology

Mice were transcardially perfused with PBS followed by 4% paraformaldehyde. The brain was gently extracted from the skull and post-fixed in 4% paraformaldehyde for 1-4 hours at room temperature or overnight at 4° C. Fixed brains were transferred to a 1% polyvinylpyrrolidone (PVP-40), 30% sucrose, 30% ethylene glycol PBS-based solution and stored at 4° C. Before slicing, brains were moved to 30% sucrose-PBS solution and rotated 24-48 hours at 4° C. to allow cryoprotectant solution to diffuse out. Brains were sliced (coronally) to 50 μm thickness using a freezing microtome. Staining of SPNs was performed with primary antibody rabbit anti-DARPP-32 (1:500, Abcam ab40801 [EP720Y]), followed by the secondary antibody AlexaFluor568 (1:500, goat anti-rabbit IgG, Invitrogen A11011). All antibodies were used according to the protocols that have been validated by suppliers. Slice imaging was performed using an Olympus FV3000 scanning confocal microscope equipped with 405, 488, 561, and 640 nm solid state diode lasers and a 20×NA0.45 air objective lens (LUCPLFN20X; Olympus), controlled by Fluoview FV31-SW software. Acquired images were analyzed in Fiji/ImageJ.

### Statistics and Reproducibility

Unless otherwise specified, all between-group statistics shown in violin plots were conducted using a two-sample independent student t-test. For within group statistics, a one-sample student t-test was used. A p-value threshold of p≤0.05 was used to determine significance. Neurons that did not meet criteria for sufficient numbers of spikes, low vs. high movement periods, or movement transitions were excluded from relevant statistical analyses.

### Data Availability

The source data for all relevant statistics and example recordings are provided in the Source Data file, and are also available at the Gihub repository:
https://github.com/HanLabBU/Shroff-Lowet-Nature-Communication-2023 (DOI:
https://zenodo.org/record/7805663 #.ZC8kEXYpD30). The experimental raw data that support the findings of this study are available from the lead contact upon request.

### Code Availability

Codes used for data analysis is available at the Github repository:
https://github.com/HanLabBU/Shroff-Lowet-Nature-Communication-2023 (DOI:
https://zenodo.org/record/7805663#.ZC8kEXYpD30) and in Supplementary Software 1. Any additional information required to reanalyze the data reported in this paper is available from the lead contact upon request.

All references cited herein are incorporated by reference, as though fully set forth herein. All orientations and arrangements of the components shown herein are used by way of example only. Further, it will be appreciated by those of ordinary skill in the pertinent art that the functions of several elements may, in alternative embodiments, be carried out by fewer elements or a single element. Similarly, in some embodiments, any functional element may perform fewer, or different, operations than those described with respect to the illustrated embodiment. Also, functional elements shown as distinct for purposes of illustration may be incorporated within other functional elements in a particular implementation.

While the subject technology has been described with respect to certain embodiments, those skilled in the art will readily appreciate that various changes and/or modifications can be made to the subject technology without departing from the spirit or scope of the subject technology. For example, each claim may depend from any or all claims in a multiple dependent manner even though such has not been originally claimed.

What is claimed is:

1. A method of improving gait in a subject, the method comprising:
exposing the subject to one or more acoustic stimuli having a frequency of 5 Hz to 30 Hz while the subject is engaging in an activity involving a repetitive physical motion performed at a frequency of less than 5 Hz.

2. The method of claim 1, wherein the one or more acoustic stimuli have a frequency of 8 Hz to 14 Hz.

3. The method of claim 1, further comprising restricting acoustic stimuli at frequencies less than 5 Hz and greater than 30 Hz from the subject while the subject is exposed to the one or more acoustic stimuli.

4. The method of claim 1, wherein the activity is walking.

5. The method of claim 1, wherein the activity involving a repetitive physical motion is performed at a frequency of approximately 0.5-4 Hz.

6. The method of claim 1, wherein the method decreases a level of variation in gait of the subject by at least 5%.

7. The method of claim 6, wherein the level of variation in gait is measured by comparing a difference in at least one of: velocity, stride length, stride width, cadence, gait phases, and electrical activity produced by muscles in the subject.

8. The method of claim 1, wherein the subject is a human.

9. The method of claim 1, wherein the one or more acoustic stimuli are delivered to the subject via headphones.

10. The method of claim 1, wherein the one or more acoustic stimuli are delivered to the subject in a pattern in which there is a first period of time with acoustic stimuli followed by a second period of time without the acoustic stimuli.

11. The method of claim 10, wherein the first period of time is equal to the second period of time.

12. The method of claim 10, wherein the first period of time is unequal to the second period of time.

13. The method of claim 10, wherein during the second period of time, no acoustic stimuli are delivered to the subject.

14. The method of claim 1, wherein the subject has been diagnosed with a neurodegenerative disease.

15. The method of claim 14, wherein the neurodegenerative disease is multiple sclerosis, essential tremor, amyotrophic lateral sclerosis, or Parkinson's disease.

16. The method of claim 1, wherein the patient suffers from: one or more myelopathies, spinal amyotrophy, cerebellar ataxia, brain tumor, craneoencephalic trauma, neuromuscular disease, one or more cerebrovascular pathologies, dementia, heart disease, and/or physiological ageing.

17. A method of improving neural activity in a subject, the method comprising:
exposing the subject to audio, visual, tactile, and/or vibrational sensory stimuli at beta frequencies and/or gamma frequencies to promote neural activity in the subject at delta frequencies.

18. The method of claim 17, wherein the subject is exposed to the audio and/or visual stimuli while the subject is engaging in an activity involving a repetitive physical motion performed at delta frequencies.

19. The method of claim 17, wherein the beta frequencies are from 10 Hz-35 Hz.

20. The method of claim 17, wherein the gamma frequencies are greater than 35 Hz.

21. The method of claim 17, wherein the delta frequencies are from 0.5 Hz-4 Hz.

22. The method of claim 17, wherein the delta frequencies are coordinated with cadence or stepping cycles of the subject.

23. The method of claim 17, wherein the method promotes locomotion of the subject.

24. The method of claim 17, wherein the method decreases a level of variation in gait of the subject by at least 5%.

25. The method of claim 24, wherein the level of variation in gait is measured by comparing a difference in at least one of: velocity, stride length, stride width, cadence, gait phases, and electrical activity produced by muscles in the subject.

26. The method of claim 17, wherein the subject does not have a diagnosed neurodegenerative condition and the method promotes athletic training in the subject.

27. The method of claim 17, wherein the subject has been diagnosed with Parkinson's disease and the method improves motor function and/or cognitive function of the subject.

28. A device for improving locomotion of a user, the device comprising:
a speaker configured to deliver auditory stimuli to the user;
a light source configured to deliver visual stimuli to the user; and
a tactile stimulator configured to deliver vibrational sensory stimulation to the user,
wherein the auditory stimuli, the visual stimuli, and the vibrational sensory stimulation are delivered at beta frequencies and/or gamma frequencies to promote gait through enhancing neural activity of the user at delta frequencies.

29. The device of claim 28, further comprising:
one or more sensors configured to measure a locomotion pattern of the user; and
a software package arranged to receive information from the one or more sensors regarding the locomotion patterns of the user and to generate a stimulation protocol based on the locomotion pattern.

30. The device of claim 28, wherein the speaker comprises a headphone.
